(19) United States
(12) Patent Application Publication
(10) Pub. No.: US 2026/0071273 A1
(43) Pub. Date: Mar. 12, 2026

(54) METHODS FOR NUCLEIC ACID SEQUENCING

(71) Applicant: NanoHyb, LLC, Pleasant Grove, UT (US)

(72) Inventors: Jonathon Hill, Heber City, UT (US); Timothy Jenkins, Heber City, UT (US); Andrew Jenkins, Heber City, UT (US)

(21) Appl. No.: 19/326,532

(22) Filed: Sep. 11, 2025

Related U.S. Application Data
(60) Provisional application No. 63/693,700, filed on Sep. 11, 2024.
(60) Provisional application No. 63/774,648, filed on Mar. 19, 2025.

(51) Int. Cl. C12Q 1/6874

(52) U.S. Cl. CPC C12Q 1/6874

(57) ABSTRACT

Provided herein, inter alia, are methods for sequencing a nucleic acid (e.g., a target polynucleotide). In addition, provided herein are methods for detecting a base modification in polynucleotide. The methods provided herein are useful for the efficient sequencing of biological samples.

### RELATED APPLICATIONS

This patent application claims the benefit of priority under 35 U.S.C. § 119 (c) of U.S. Provisional Patent Application Nos. 63/693,700 filed on Sep. 11, 2024, and 63/774,648 filed on Mar. 19, 2025, each of which is hereby incorporated herein by reference in its entirety and for all purposes.

### BACKGROUND

Quantifying the location and frequency of base modifications in the genome, especially methylated cytosines, is an essential tool for studying gene regulation, epigenetic inheritance, and other biological processes, as well as diagnosing disease in the clinic. The present disclosure addresses these and other concerns in the art.

### SUMMARY

Current array-based methods for identifying base modifications in a genome or other nucleic acid sequence, especially methylated cytosines, are expensive, have limited accuracy and dynamic range, and cannot identify other base modifications or determine the type of methylation present. In response, researchers in the lab and the clinic are increasingly moving to reduced representation bisulfite sequencing (RRBS) or reduced representation methylation-sequencing (RRMS). However, RRBS is also technically limited due to the difficulty and inconsistency of enrichment at regions of interest using bisulfite-converted DNA. Additionally, RRMS is often not a realistic alternative as it requires large amounts of sequencing because the whole genome must be sequenced. Thus, improved methods are provided that allow for targeted assessment of modified bases at specific regions of a sequence, e.g., a genomic region, in a cost-effective and accurate manner. Ideally, in at least some embodiments this technique avoids the two major contributors to assay variance: amplification, and bisulfite (or enzymatic) conversion of unmethylated cytosines. Such improvements are detailed herein.

In an aspect is provided a method for sequencing a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with a first polymerase under conditions promoting creation of a complementary adapter-target polynucleotide, wherein the complementary adapter-target polynucleotide includes a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing includes sequencing of the target polynucleotide sequence without sequencing the complementary adapter-target polynucleotide, thereby sequencing the target polynucleotide, optionally wherein the target polynucleotide includes one or more base modifications.

In another aspect is provided a method for detecting a base modification in a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with a first polymerase under conditions promoting creation of a complementary adapter-target polynucleotide, wherein the complementary adapter-target polynucleotide includes a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing includes sequencing of the target polynucleotide sequence without sequencing the complementary adapter-target polynucleotide, thereby detecting the base modification in the target polynucleotide.

In another aspect is provided a method for quantifying a base modification in a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with a first polymerase under conditions promoting creation of a complementary adapter-target polynucleotide, wherein the complementary adapter-target polynucleotide includes a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing includes sequencing of the target polynucleotide sequence without sequencing the complementary adapter-target polynucleotide, thereby quantifying the base modification in the target polynucleotide.

In another aspect is provided a method for sequencing a plurality of target polynucleotides, the method including: (a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters include a primer binding site; (b) contacting the plurality of adapter-target polynucleotides with a first polymerase under conditions promoting creation of a plurality of complementary adapter-target polynucleotides, wherein each of the complementary adapter-target polynucleotides includes a 3′ protective group or a 5′ protective group; (c) contacting the plurality of adapter-target polynucleotides with a sequencing adapter in the presence of a second ligase under conditions promoting ligation of the sequencing adapter to the adapter-target polynucleotide to create a plurality of sequencing complexes; and (d) sequencing the plurality of sequencing complexes, wherein the sequencing includes sequencing of the plurality of the target polynucleotide sequences without sequencing the plurality of complementary adapter-target polynucleotides, thereby sequencing the plurality of target polynucleotides, optionally wherein the plurality of target polynucleotides includes one or more base modifications.

In another aspect is provided a method for detecting a plurality of base modifications in a plurality of target polynucleotides, the method including: (a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters include a primer binding site; (b) contacting the plurality of adapter-target polynucleotides with a first polymerase under conditions promoting creation of a plurality of complementary adapter-target polynucleotides, wherein each of the complementary adapter-target polynucleotides includes a 3′ protective group or a 5′ protective group; (c) contacting the plurality of adapter-target polynucleotides with a sequencing adapter in the presence of a second ligase under conditions promoting ligation of the sequencing adapter to the adapter-target polynucleotide to create a plurality of sequencing complexes; and (d) sequencing the plurality of sequencing complexes, wherein the sequencing includes sequencing of the plurality of the target polynucleotide sequences without sequencing the plurality of complementary adapter-target polynucleotides, thereby detecting the plurality of base modifications in the plurality of target polynucleotides.

In another aspect is provided a method for detecting a plurality of base modifications in a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with a first polymerase under conditions promoting creation of a complementary adapter-target polynucleotide, wherein the complementary adapter-target polynucleotide includes a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing includes sequencing of the target polynucleotide sequence without sequencing the complementary adapter-target polynucleotide, thereby detecting the plurality of base modifications in the target polynucleotide.

In another aspect is provided a method for detecting a base modification in a plurality of target polynucleotides, the method including: (a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters include a primer binding site; (b) contacting the plurality of adapter-target polynucleotides with a first polymerase under conditions promoting creation of a plurality of complementary adapter-target polynucleotides, wherein each of the complementary adapter-target polynucleotides includes a 3′ protective group or a 5′ protective group; (c) contacting the plurality of adapter-target polynucleotides with a sequencing adapter in the presence of a second ligase under conditions promoting ligation of the sequencing adapter to the adapter-target polynucleotide to create a plurality of sequencing complexes; and (d) sequencing the plurality of sequencing complexes, wherein the sequencing includes sequencing of the plurality of the target polynucleotide sequences without sequencing the plurality of complementary adapter-target polynucleotides, thereby detecting the base modification in the plurality of target polynucleotides.

In another aspect is provided a method for sequencing a target polynucleotide, the method including: (a) dephosphorylating the target polynucleotide; (b) contacting the target polynucleotide with one or more adapters in the presence of a first ligase under conditions promoting ligation of the one or more adapters to the target polynucleotide to create an adapter-target polynucleotide, wherein the one or more adapters optionally include a primer binding site, a first portion of a cognate binding pair, and/or a uridine; (c) contacting the adapter-target polynucleotide with a solid support including a second portion of a cognate binding pair under conditions allowing the binding of the first portion of a cognate binding pair and a second portion of a cognate binding pair to create a pulldown complex; (d) contacting the adapter-target polynucleotide with a first polymerase under conditions promoting creation of a complementary adapter-target polynucleotide, wherein the complementary adapter-target polynucleotide includes a 3′ protective group or a 5′ protective group; (c) optionally contacting the adapter-target polynucleotide and complementary adapter-target polynucleotide with one or more barcodes or ligation adapters in the presence of a second ligase under conditions promoting ligation of the one or more barcodes or ligation adapters to the adapter-target polynucleotide and complementary adapter-target polynucleotide; (f) optionally the pulldown complex is isolated; (g) the pulldown complex is contacted with a uridine-targeted enzyme to excise a uridine residue and elute the adapter-target polynucleotide and complementary adapter-target polynucleotide from the solid support, thereby forming a sequencing complex; and (h) sequencing the sequencing complex, wherein the sequencing includes sequencing of the adapter-target polynucleotide sequence without sequencing the complementary adapter-target polynucleotide, thereby sequencing the target polynucleotide, optionally wherein the target polynucleotide includes one or more base modifications.

### BRIEF DESCRIPTION OF THE DRAWINGS

FIG. 1 shows an example method of sequencing a target polynucleotide described herein. Steps 1-2: Target polynucleotide (e.g., cfDNA) is dephosphorylated and a 3′ ligation step adds a known adapter (e.g., oligonucleotide) to the end of the target polynucleotide (e.g., native DNA or native strand). Steps 3-4: Second strand synthesis is carried out using a primer complementary to the ligated adapter (e.g., oligonucleotide). Step 5: Barcodes and ligation adapters are added to the 5′ end of the native strand. The adapter-target polynucleotide (e.g. native DNA or native strand) is then sequenced, but the complementary adapter-target polynucleotide (e.g., synthetic strand or second strand) is not sequenced.

FIG. 2 shows an example method of sequencing a target polynucleotide described herein. Steps 1-2: Target polynucleotide (e.g., cfDNA) is dephosphorylated and a 3′ ligation step adds an adapter (e.g. oligonucleotide) including a first portion of a cognate binding pair (e.g., bead attachment component) and a uridine to the 3′ end of the target polynucleotide (e.g., native DNA or native strand). Steps 3-4: The adapter-target polynucleotide is contacted with a solid support (e.g., bead) including a second portion of a cognate binding pair under conditions allowing hybridization or binding of the first portion of a cognate binding pair and a second portion of a cognate binding pair to create a pulldown complex (e.g., biotin-streptavidin binding or click chemistry). Second strand synthesis is carried out using a primer complementary to the ligated adapter (e.g., oligonucleotide). Step 5: Barcodes and ligation adapters are added to the adapter-target polynucleotide and complementary adapter-target polynucleotide while part of the pulldown complex. Optionally the pulldown complex is isolated. Step 6: The adapter-target polynucleotide and complementary adapter-target polynucleotide is cleaved off of the solid support via a uridine-targeted enzyme (e.g., USER enzyme). Step 7: The adapter-target polynucleotide (e.g. native DNA or native strand) is then sequenced, but the complementary adapter-target polynucleotide (e.g., synthetic strand or second strand) is not sequenced.

FIG. 3 shows an example method described herein. Step 1: Adapters (e.g., oligonucleotide probes) are designed to be complementary to specific regions of interest within a target polynucleotide or genome. The adapters include priming points or primer binding sites at either the 5′ or 3′ end for extension (e.g. second strand synthesis or PCR amplification). Step 2: Amplify (e.g. extend) the adapters to produce amplicon adapters. The amplification of the adapters includes a Q5U polymerase and a deoxynucleotide triphosphate mix that includes deoxyuridine triphosphate, but excludes deoxythymidine triphosphate. The amplicon adapters include a uridine and do not include a thymidine. The amplicon adapters include a first portion of a cognate binding pair (e.g., biotin) for pulldown and/or isolation. Step 3: The target polynucleotide is contacted with a kinase (e.g., T4 polynucleotide kinase) to remove 3′ phosphate groups and add 5′ phosphate groups. Then, target polynucleotide is denatured. Step 4: Amplicon adapters are allowed to hybridize overnight with the target polynucleotide of Step 3, and the adapter-target polynucleotide is then contacted with a solid support (e.g., bead) including a second portion of a cognate binding pair (e.g., streptavidin) to form a pulldown complex. The pulldown complex is isolated and washed to remove any excess sequences. Step 5: The pulldown complex is contacted with a uridine-targeting enzyme (e.g., USER II) to excise the uridine in the amplicon adapters, which degrades the amplicon adapters and elutes them from the pulldown complex and/or solid support. Step 6: The target polynucleotide is exposed to heat to inactivate the uridine-targeting enzyme and elute the target polynucleotide from the solid support and/or pulldown complex. Optionally an adapter (e.g. original adapter) may remain bound for second strand synthesis.

FIG. 4 shows an example method described herein. Step 1: Previously primed target polynucleotides are incubated with a first polymerase (e.g., DNA Pol I) and a ligase (e.g., E. coli ligase) to generate a complementary target polynucleotide with a completed 3′ end which is ready for library prep. Step 2: The target polynucleotide (e.g., native strand) and the complementary target polynucleotide (e.g. synthetic strand or second strand) are A-tailed on the 3′ end of the complementary target polynucleotide. Step 3: Adapters are ligated to the 5′ end of the target polynucleotide but not the 3′ end overhang. Step 4: Only the target polynucleotide (e.g. native strand) is sequenced because the complementary target polynucleotide (e.g. synthetic strand or second strand) remains unfinished and without an adapter.

FIGS. 5A-5C show results from training models for unique methylation patterns in cortical neurons (FIG. 5A), dopaminergic neurons (FIG. 5B), and spinal motor neurons (FIG. 5C).

FIGS. 6A-6C show sequencing results from the method described herein to quantify neuronal methylation patterns in cell free DNA (cfDNA) in subjects with neurodegenerative diseases. FIG. 6A shows a quantification of cortical neuron methylation patterns in cfDNA from subjects with Alzheimer's disease, compared to subjects with mild cognitive impairment and control subjects. FIG. 6B shows a quantification of spinal motor neuron methylation patterns in cfDNA from subjects with either definite or probable amyotrophic lateral sclerosis (ALS), compared to subjects with Parkinson's disease and control subjects. FIG. 6C shows a breakdown of the number of samples analyzed for each group in FIGS. 6A-6B.

FIG. 7 shows an example method described herein. The method includes hybridization of an isolation probe to a target polynucleotide. The isolation probe includes a first portion of a cognate pair non-covalently bound to a solid support including a second portion of the cognate binding pair, thereby forming a pulldown complex. The pulldown complex includes the isolation probe hybridized to the target polynucleotide and the isolation probe hybridized to the solid support (top strand schematic). After isolating the pulldown complex, the isolation probes are denatured (middle strand schematic). After the denaturing of the isolation probes, second strand synthesis is performed by using a barcode sequence within the adapter added to the target polynucleotide (bottom strand schematic).

FIG. 8 shows an example method described herein. DNA (e.g., target polynucleotide) is extracted from a sample, denatured, and end repaired (phosphates are removed from 3′ ends and added to 5′ ends). This repaired DNA is then treated with TdT where an arbitrary number of bases are added, generating a homopolymer tail. A complementary homopolymer primer with or without a barcode sequence is added to the TdT treated sample and second strand synthesis takes place as described previously. The ends can now be distinguished from each other based on the barcode sequence, or if that sequence was omitted, the DNA will be left with only one end capable of further ligation and sequencing, allowing only the native strand to be sequenced.

FIG. 9 shows an example method using random hexamers described herein. The method includes the use of random hexamers during second strand synthesis. The method includes contacting a target polynucleotide with one or more random hexamer probes, to create a hexamer probe-target polynucleotide complex (top strand schematic). Isolation probe fragments may still be hybridized to the target polynucleotide. Then, the hexamer probe-target polynucleotide complex is contacted with a polymerase (e.g., DNA Pol I) and a ligase (e.g., E. coli ligase) for creation of a synthetic complementary target polynucleotide (middle strand schematic). The polymerase allows for creation of the complementary target polynucleotide and the ligase repairs any nicks in the target polynucleotide (e.g., native strand). Following second strand synthesis, the target polynucleotide and the complementary target polynucleotide are further processed using library preparation methods provided herein (bottom strand schematic).

FIG. 10 shows an example method of amplifying isolation probes described herein. The original probe is contacted by a polymerase (e.g., Q5U polymerase) in the presence of a plurality of deoxynucleotide triphosphates (dNTPs) which include deoxyuridine triphosphate (dUTP) and do not include deoxythymidine triphosphate (dTTP), thereby creating an isolation probe. This amplification process is repeated to create a plurality of isolation probes. Each of the isolation probes are biotinylated on the 5′ end.

FIG. 11 shows an example method for removing isolation probes from a target polynucleotide (e.g., native strand) provided herein. The exemplified method includes contacting an isolation probe-target polynucleotide complex with a solid support. The isolation probe includes a first portion of a cognate binding pair (e.g., biotin) and the solid support includes a second portion of the cognate binding pair (e.g., streptavidin). The first and second portion of the cognate binding pair are capable of forming a non-covalent bond, thereby forming a pulldown complex. The pulldown complex is the isolated. After washing out the untargeted strands, the pulldown complex is contacted by a non-standard DNA nucleotide-targeting enzyme (e.g., USER II enzyme). The non-standard DNA nucleotide-targeting enzyme excises the uridine nucleotides in the one or more isolation probes, creating gaps throughout the isolation probes (e.g., isolation probe fragments). The gaps created lead to degradation of the probes. Then, the non-standard DNA nucleotide-targeting enzyme is heat-inactivated, resulting in the isolation probe fragments to denature from the target polynucleotide.

FIG. 12 shows an example method for removing isolation probes from a target polynucleotide (e.g., native strand) provided herein. The exemplified method uses RNA-based isolation probes capable of hybridizing to a complementary region of the target polynucleotide, to form an isolation probe-target polynucleotide complex. The RNA-based isolation probes include a first portion of a cognate binding pair (e.g., biotin). The isolation probe-target polynucleotide complex is contacted by a solid support including a second portion of the cognate binding pair (e.g., streptavidin). The first and second portion of the cognate binding pair are capable of forming a non-covalent bond, thereby forming a pulldown complex. The pulldown complex is then isolated. Once the non-target polynucleotide strands are removed, the pulldown complex is contacted with an RNase enzyme. The RNase enzyme digests the RNA-based isolation probes, thereby eluting the target polynucleotide from the isolation probes and solid support. The target polynucleotide may then be used in any of the methods provided herein for second strand synthesis and/or sequencing.

FIGS. 13A-13B show that the methods described herein can be used to analyze sequence variation and/or base modification (e.g., methylation) of different corn varieties. FIG. 13A: Genomic pileup at a targeted region showing methylated CpG sites (gray) and unmethylated CpG sites (black). The average methylation across these islands distinguished between two different corn varieties in which one type has higher methylation (top) than the second type (bottom). FIG. 13B: Little to no off-target sequencing occurred in regions adjacent to the targeted region of the genome, which demonstrated the enrichment of for the targeted polynucleotide was efficient.

FIGS. 14A-14B show that the methods described herein can be used to analyze sequence variation and/or base modification (e.g., methylation) of genes associated with Polycystic Kidney Disease (PKD). FIG. 14A: Methylated (gray) and unmethylated (black) regions in the PKD1 gene. FIG. 14B: Targeting exons of the PKD2 gene showed little to no background for non-targeted regions.

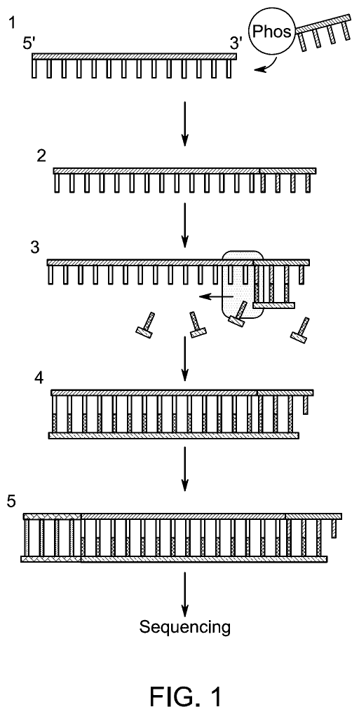

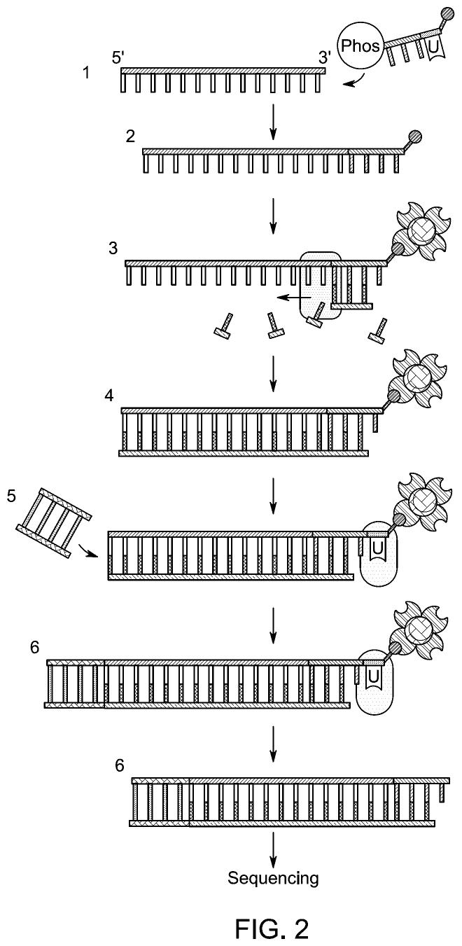

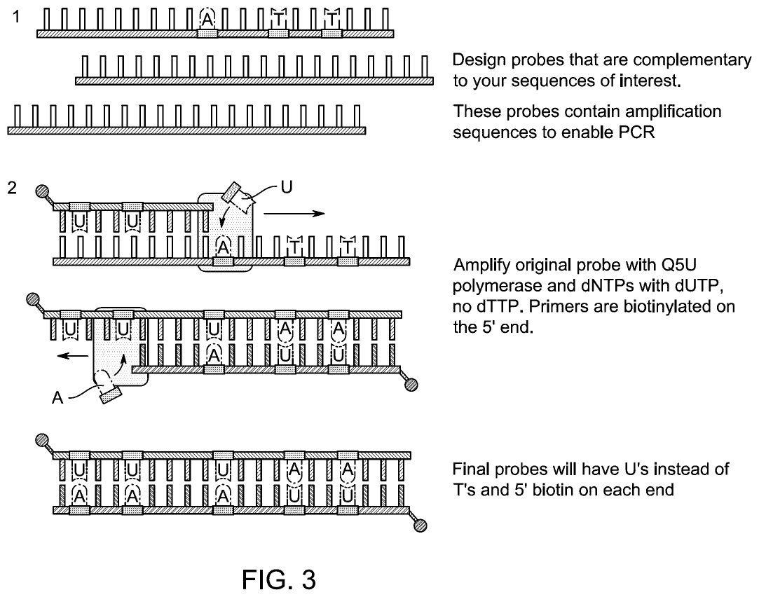

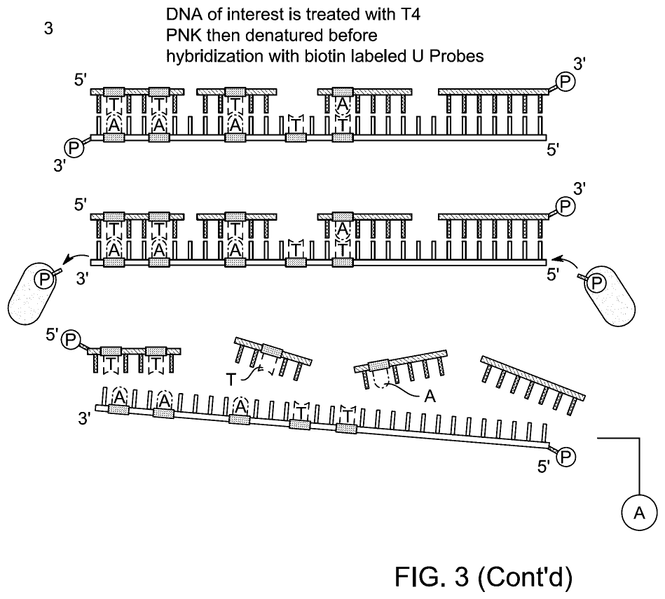

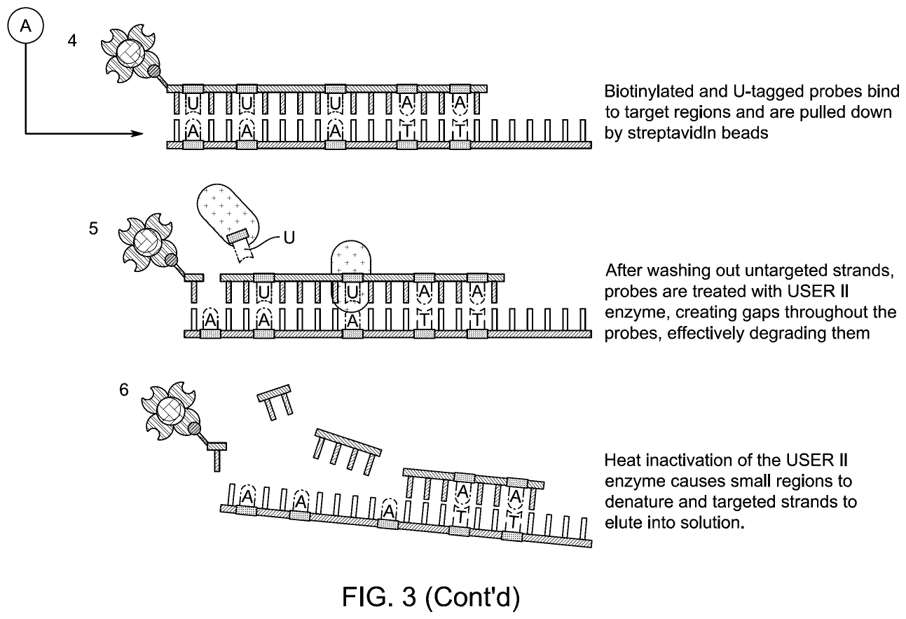

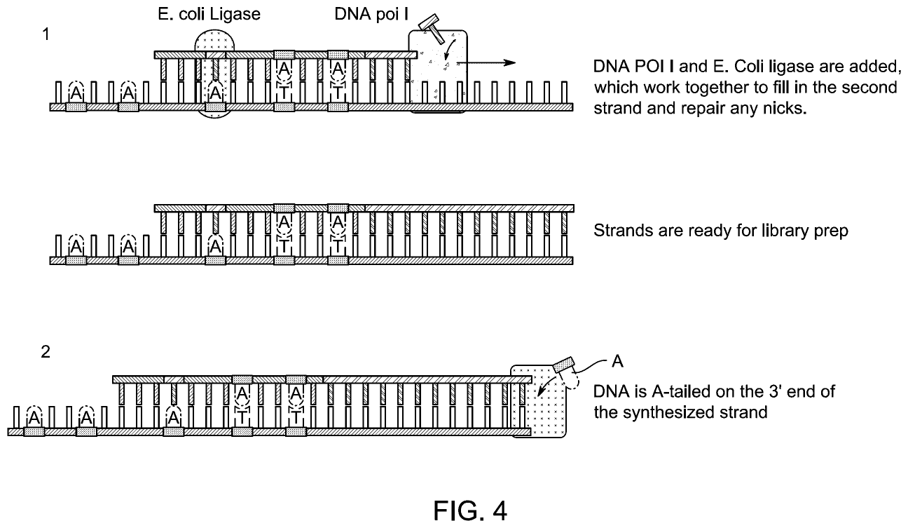

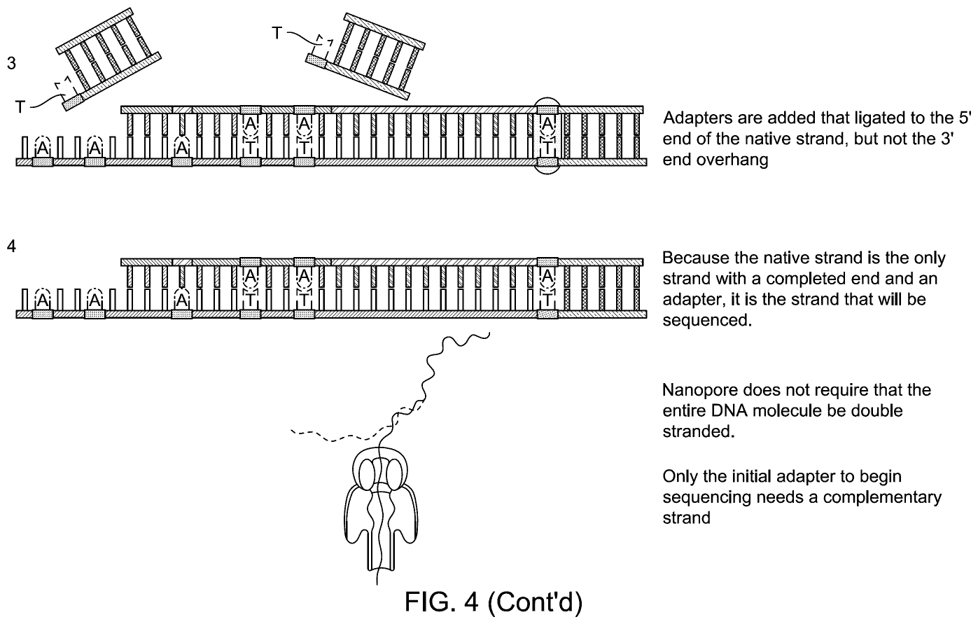

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

As used herein, the term “each,” when used in reference to a collection of items, is intended to identify an individual item in the collection but does not necessarily refer to every item in the collection. Exceptions can occur if explicit disclosure or context clearly dictates otherwise.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The term “nucleotide” according to the present disclosure particularly relates to ribonucleotides, 2′-deoxyribonucleotides or 2′,3′-dideoxyribonucleotides.

The term “nucleobase” refers to either native or non-native purine or pyrimidine bases. Nucleobases include adenine, cytosine, guanine, thymine, uracil, hypoxanthine, xanthine, 7-deaza-adenine and 7-deazaguanine, inosine.

As used herein, the phrase “dNTP” means 2′-deoxynucleotidetriphosphate, where the nucleotide comprises a native or non-native nucleobase.

As used herein, the term “double-stranded,” when used in reference to a polynucleotide, means that some or all of the nucleotides between complementary strands of a polynucleotide are hydrogen bonded together to form a partial or complete double helix. A partially double stranded polynucleotide can have at least 10%, 25%, 50%, 60%, 70%, 80%, 90% or 95% of its nucleotides hydrogen bonded to a complementary nucleotide.

A single-stranded polynucleotide refers to a polynucleotide that has few to no hydrogen bonds with another polynucleotide such that a double helix is not formed or is unstable under a given set of hybridization conditions.

A “polymerase” is generally an enzyme that catalyzes the reaction between 3′-OH and 5′-triphosphate in nucleotides, oligomers, and their analogs to form nucleic acid polymers. Polymerases include, but are not limited to, DNA-dependent DNA polymerases, RNA-dependent DNA polymerases, template-independent DNA polymerase, T7 DNA polymerase, T3 DNA polymerase, T4 DNA polymerase, DNA polymerase 1, Klenow fragment, Thermophilus aquaticus DNA polymerase, Tth DNA polymerase, Phusion DNA Polymerase, SuperFi DNA Polymerase, Vent DNA polymerase, Deep Vent DNA polymerase, Bst DNA Polymerase Large Fragment, Stoeffel Fragment, 9° N DNA Polymerase, Pfu DNA Polymerase, Tfl DNA Polymerase, Phi29 Polymerase, Tli DNA polymerase, eukaryotic DNA polymerase beta, telomerase, KOD HiFi, KODI DNA polymerase, Q-beta replicase, terminal transferase (TdT), AMV reverse transcriptase, M-MLV reverse transcriptase, Phi6 reverse transcriptase, HIV-1 reverse transcriptase, Thermo Sequenase (Thermo Fisher Scientific). These polymerases include wild-type, mutant isoforms, chimeric forms, and genetically engineered variants such as exo-polymerases and other mutants, e.g., that tolerate modified nucleotides and incorporate them into a strand of nucleic acid.

As used herein, the term “Q5U polymerase” refers to a hot start high-fidelity DNA polymerase that is thermostable and includes 3′ to 5′ exonuclease activity. In some embodiments, it is fused to a processivity-enhancing Sso7d domain. Other similar polymerases may be contemplated where a Q5U polymerase is recited herein.

As used herein, the term “base modification” refers to a nucleotide that is modified through the addition of chemical groups or substituted with a non-standard nucleotide or non-standard nucleoside. In embodiments, the non-standard nucleotide is a non-natural nucleotide. In embodiments, the non-standard nucleoside isa non-natural nucleoside. In embodiments, the base modification includes methylation. In embodiments, the base modification includes a n1-methyl-pseudouridine, a pseudouridine (Ψ), 5-methylcytosine (5mC), a 4-methylcytosine (4mC), an N 6-methyladenine (6 mA), an N 6-methyladenosine (m6a), an N 1-methyladenosine (m1aA), a 7-methylguanine (m7G), or 2′-O-methylation (2′-O-Methyl). In embodiments, the base modification includes oxidation of a methylated nucleotide. In embodiments, the base modification includes a 5-hydroxymethylcytosinc (5hmC), a 5-formylcytosine (5fC), a 5-carboxylcytosine (5caC), a 5-hydroxymethyluracil (5hmU), a 5formyluracil (5hmU), or a β-D-glucosyl-hydroxymethyluracil (base J). In embodiments, the base modification includes a bromodeoxyuridine (BrdU). In embodiments, the base modification includes a 5-bromo-2′-deoxyuridine (BrdU) or a 5-ethynyl-2′-deoxyuridine (ErdU).

As used herein, the term “methylation” refers to the process by which methyl groups are added to a nucleotide. In embodiments, methylation includes addition of methyl groups to adenine, cytosine, or guanine. In embodiments, methylation includes 2′-O-methylation (2′-O-Methyl).

As used herein, the term “non-natural nucleotide” refers to nucleotide analogs, synthetic nucleotides, and nucleotide mimetics which are not found in nature. In embodiments, a non-natural nucleotide can replace a natural nucleotide found in a polynucleotide. In embodiments, a non-natural nucleotide can substitute a natural nucleotide found in a polynucleotide.

As used herein, the term “non-natural nucleoside” refers to nucleoside analogs, synthetic nucleosides, and nucleoside mimetics which are not found in nature. In embodiments, a non-natural nucleoside can replace a natural nucleoside found in a polynucleotide. In embodiments, a non-natural nucleoside can substitute a natural nucleoside found in a polynucleotide. In embodiments, the non-natural nucleoside is bromodeoxyuridine (BrdU). In embodiments, the non-natural nucleoside is 5-bromo-2′-deoxyuridine (BrdU). In embodiments, the non-natural nucleoside is 5-ethynyl-2′-deoxyuridinc (ErdU). In embodiments, the non-natural nucleoside is 2′-Deoxy-5-ethynyluridine (ErdU).

As used herein, the term “target polynucleotide” refers to a polynucleotide that is selected for sequencing. For example, and without limitation, the target polynucleotide may be a polynucleotide having or expected to have a base modification. In embodiments, the target polynucleotide may be a polynucleotide having or expected to have a base modification in a given cell type. In embodiments, the target polynucleotide may be a genomic polynucleotide. In embodiments, the target polynucleotide may be genomic DNA. In embodiments, the target polynucleotide may be part of the genome of a cell. In embodiments, the target polynucleotide may be a fragment of the genome of a cell. In embodiments, the target polynucleotide may be DNA from a particular cell type. In embodiments, the target polynucleotide may be used to identify a cell type. In embodiments, the target polynucleotide may be from any compartment within a cell. In embodiments, the target polynucleotide may be a nuclear polynucleotide. In embodiments, the target polynucleotide may be nuclear DNA. In embodiments, the target polynucleotide may be a mitochondrial polynucleotide. In embodiments, the target polynucleotide may be mitochondrial DNA (mDNA). In embodiments, the target polynucleotide may be part of the mitochondrial DNA of a cell. In embodiments, the target polynucleotide may be a mitochondrial DNA fragment. In embodiments, the target polynucleotide may be a DNA fragment. In embodiments, the target polynucleotide may be chromosomal DNA or a fragment thereof. In embodiments, the target polynucleotide may be outside a cell. In embodiments, the target polynucleotide may be cell-free DNA (cfDNA). In embodiments, the target polynucleotide may be included in a biological sample. In embodiments, the target polynucleotide may be isolated from a biological sample. In embodiments, the target polynucleotide may be DNA from an animal. In embodiments, the target polynucleotide may be DNA from a vertebrate animal. In embodiments, the target polynucleotide may be DNA from a mammal. In embodiments, the target polynucleotide may be DNA from a human. In embodiments, the target polynucleotide may be DNA from a rodent. In embodiments, the target polynucleotide may be DNA from a mouse. In embodiments, the target polynucleotide may be DNA from a rat. In embodiments, the target polynucleotide may be DNA from an invertebrate animal. In embodiments, the target polynucleotide may be DNA from a plant. In embodiments, the target polynucleotide may be DNA from a pathogen. In embodiments, the target polynucleotide may be DNA from a virus. In embodiments, the target polynucleotide may be DNA from bacteria. In embodiments, the target polynucleotide may be DNA from a parasite. In embodiments, the target polynucleotide may be DNA from an archacon.

As used herein, the term “adapter” refers generally to any oligonucleotide that can be added, for example, ligated, to a nucleic acid molecule, thereby generating nucleic acid products that can be sequenced on a sequencing platform. In some embodiments, adapters include two complementary oligonucleotides forming a double-stranded structure. In embodiments, the adapter is an oligonucleotide that can be ligated to the end of a target polynucleotide. In embodiments, the adapter includes DNA. In embodiments, the adapter includes RNA.

As used herein, the term “isolation probe” refers generally to any biomolecule (e.g., oligonucleotide) capable of hybridizing with or binding to a nucleic acid (e.g., target polynucleotide). In some embodiments, isolation probes may be used in a method of isolating a nucleic acid (e.g., target polynucleotide). In some embodiments, isolation probes may be used in an isolation step in a method provided herein. In some embodiments, isolation probes may form part of a pulldown complex.

As used herein, the term “ligation” refers to the process of joining two biomolecules (e.g., oligonucleotides, polynucleotides) by forming a chemical bond between their ends. In embodiments, the chemical bond is a non-covalent chemical bond. In embodiments, ligation is catalyzed by a ligase.

As used herein, the term “ligase” refer to an enzyme that catalyzes the ligation of two biomolecules (e.g., oligonucleotides, polynucleotides) by forming a new chemical bond.

As used herein, the term “adapter-target polynucleotide” refers to a target polynucleotide which has one or more adapters ligated to an end (or both ends) of the target polynucleotide.

As used herein, the term “DNA end-repair composition” refers generally to a composition (e.g., buffer) including one or more components capable of modifying the 5′ end or the 3′ end of a nucleic acid (e.g., DNA, target polynucleotide, etc.). In some embodiments, the DNA end-repair composition is capable of modifying the 5′ end or the 3′ end of a nucleic acid to produce ends that are compatible for subsequent enzymatic reactions, such as ligation or adapter addition. In some embodiments, the DNA end-repair composition is capable of modifying the 5′ end or the 3′ end of a nucleic acid (e.g., DNA, target polynucleotide, etc.) to produce a blunt-ended nucleic acid. In some embodiments, the DNA end-repair composition is capable of repairing a nucleic acid with damaged and/or an incompatible 5′ end and/or an incompatible 3′ end. In some embodiments, the DNA end-repair composition is capable of converting the incompatible 5′ end and/or the incompatible 3′ end of a nucleic acid to a 5′-phosphorylated, blunt-ended nucleic acid. In some embodiments, the DNA end-repair composition includes one or more of a DNA polymerase, an exonuclease, or an endonuclease. In some embodiments, the DNA end-repair composition includes one or more of a DNA polymerase. In some embodiments, the DNA end-repair composition includes one or more of an exonuclease. In some embodiments, the DNA end-repair composition includes one or more of an endonuclease. In some embodiments, the DNA end-repair composition includes a T4 DNA polymerase, a T4 polynucleotide kinase, and/or deoxynucleotide triphosphates (dNTPs). In some embodiments, the DNA end-repair composition includes a T4 DNA polymerase. In some embodiments, the DNA end-repair composition includes a T4 polynucleotide kinase. In some embodiments, the DNA end-repair composition includes deoxynucleotide triphosphates (dNTPs).

As used herein, the term “end-repaired target polynucleotide” refers to a target nucleotide that has been contacted and repaired by a DNA end-repair composition. In some embodiments, the end-repaired target polynucleotide includes a 5′-phosphorylated, blunt-end.

As used herein, the term “exonuclease” refers generally to an enzyme capable of cleaving one or more nucleotides from the end of a polynucleotide strand. In some embodiments, the exonuclease is capable of excising a homopolymer tail. In some embodiments the exonuclease is a 3′->5′ exonuclease. In some embodiments, the exonuclease is a Klenow exo-enzyme (e.g., Klenow fragment).

As used herein, the term “homopolymer tail” refers generally to a nucleotide sequence that includes a consecutive repetition of the same nucleotide. In some embodiments, the homopolymer tail is a polyadenine (poly-A) tail, a polycytosine (poly-C) tail, a polyguanine (poly-G) tail, or a polythymine (poly-T) tail. In some embodiments, the homopolymer tail is a polyadenine (poly-A) tail. In some embodiments, the homopolymer tail is a a polycytosine (poly-C) tail. In some embodiments, the homopolymer tail is a polyguanine (poly-G) tail. In some embodiments, the homopolymer tail is a polythymine (poly-T) tail. In some embodiments, the homopolymer tail is added to the 5′ end or the 3′ end of a nucleic acid (e.g., target polynucleotide). In some embodiments, the homopolymer tail is added to the 5′ end of the nucleic acid. In some embodiments, the homopolymer tail is added to the 3′ end of the nucleic acid.

As used herein the term “transferase” refers generally to an enzyme capable of catalyzing the transfer of specific functional groups (e.g., nucleotides) from one biomolecule to another biomolecule. In some embodiments, the transferase is capable of catalyzing the addition of one or more nucleotides to the 5′ end or the 3′ end of a nucleic acid (e.g., target polynucleotide). In some embodiments, the transferase is capable of catalyzing the addition of a homopolymer tail to a nucleic acid (e.g., target polynucleotide). In some embodiments, the transferase is a homopolymer transferase.

As used herein, the term “random hexamer primer” refers generally to an oligonucleotide sequence including 6 nucleotides which are synthesized in a random order. In some embodiments, the random hexamer primer includes 6 nucleotides selected from any combination of adenine, cytosine, guanine, thymine, and/or uracil.

As used herein, the term “primer binding site” refers to a nucleotide sequence of a polynucleotide to which a primer binds and serves as the site of initiation for replication of the polynucleotide.

As used herein, the term “complementary adapter-target polynucleotide” refers to a polynucleotide that is complementary to and hybridizes to the adapter-target polynucleotide. In embodiments, the complementary adapter-target polynucleotide includes a 3′ protective group. In embodiments, the complementary adapter-target polynucleotide includes a 5′ protective group. In embodiments, the complementary adapter-target polynucleotide is complementary to all or substantially all of the adapter-target polynucleotide. In embodiments, the complementary adapter-target polynucleotide is complementary to a portion of the adapter-target polynucleotide.

As used herein, the term “3′ protective group” refers to a moiety on the end of a polynucleotide to prevent sequencing of the polynucleotide. In embodiments, the 3′ protective group prevents sequencing of the complementary adapter-target polynucleotide. In embodiments, the 3′ protective group prevents ligation of a sequencing adapter to the complementary adapter-target polynucleotide. In embodiments, the 3′ protective group includes a nucleotide overhang or a 3′ phosphate. In embodiments, the 3′ protective group includes a nucleotide overhang. In embodiments, the 3′ protective group includes a 3′ phosphate.

As used herein, the term “5′ protective group” refers to a moiety on the 5′ end of a polynucleotide to prevent sequencing of the polynucleotide. In embodiments, the 5′ protective group prevents sequencing of the complementary adapter-target polynucleotide. In embodiments, the 5′ protective group prevents ligation of a sequencing adapter to the complementary adapter-target polynucleotide. In embodiments, the 5′ protective group includes an aldehyde, an amine, or a thiol. In embodiments, the 5′ protective group includes an aldehyde. In embodiments, the 5′ protective group includes an amine. In embodiments, the 5′ protective group includes a thiol. In embodiments, the 5′ protective group includes a 5′ aldehyde. In embodiments, the 5′ protective group includes a 5′ amine. In embodiments, the 5′ protective group includes a 5′ thiol.

As used herein, the term nucleotide overhang refers to a sequence of unpaired nucleotides at the end of a polynucleotide.

As used herein, the term “complementary” or “substantially complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed. In embodiments, a first template polynucleotide and a second template polynucleotide of an overlapping cluster are not substantially complementary (e.g., are at least 50%, 75%, 90%, or more non-complementary to each other).

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, nucleic acid, a protein, or enzyme (e.g., a DNA polymerase).

As used herein, the term “barcode” refers to a known nucleic acid sequence that allows some feature of a nucleic acid with which the barcode is associated to be identified. In some embodiments, the feature of the nucleic acid to be identified is the sample or source from which the nucleic acid is derived. By way of example only, some embodiments described herein describe the addition of multiple barcodes (e.g., 2, 3, 4, 5, 6, or more) to the nucleic acids of interest in a single cell present in a population of cells. The unique combination of barcodes added to the nucleic acids of each individual cells can advantageously enable the identification of the cell from which the tagged nucleic acid of interest was derived. In some embodiments, barcodes are at least 3, 4, 5, 6, 7, 8, 9, 10, 11, 12, 13, 14, 15, or more nucleotides in length. In some embodiments, barcodes are shorter than 10, 9, 8, 7, 6, 5, or 4 nucleotides in length. In some embodiments, barcodes associated with some nucleic acids are of a different length than barcodes associated with other nucleic acids. In general, barcodes are of sufficient length and comprise sequences that are sufficiently different to allow the identification of samples based on barcodes with which they are associated. In some embodiments, a barcode and the sample source with which it is associated can be identified accurately after the mutation, insertion, or deletion of one or more nucleotides in the barcode sequence, such as the mutation, insertion, or deletion. In some embodiments, each barcode in a plurality of barcodes differs from every other barcode in the plurality at two or more nucleotide positions, such as at 2, 3, 4, 5, 6, 7, 8, 9, 10, or more positions. In some embodiments, one or more adaptors comprise(s) at least one of a plurality of barcode sequences. In some embodiments, methods of the technology further comprise identifying the sample or source from which a target nucleic acid is derived based on a barcode sequence to which the target nucleic acid is joined. In some embodiments, methods of the technology further comprise identifying the target nucleic acid based on a barcode sequence to which the target nucleic acid is joined. Some embodiments of the method further comprise identifying a source or sample of the target nucleotide sequence by determining a barcode nucleotide sequence. Some embodiments of the method further comprise molecular counting applications (e.g., digital barcode enumeration and/or binning) to determine expression levels or copy number status of desired targets. In general, a barcode may comprise a nucleic acid sequence that when joined to a target nucleic acid serves as an identifier of the sample from which the target polynucleotide was derived.

As used herein, the term “primer” or “extension primer” refers to an oligonucleotide, whether occurring naturally or produced synthetically, that is capable of acting as a point of initiation of nucleic acid synthesis when placed under appropriate conditions, e.g., in the presence of nucleotide triphosphates and a polymerase enzyme (for example, a thermostable polymerase enzyme) in an appropriate buffer (“buffer” includes appropriate pH, ionic strength, cofactors, etc.) and at a suitable temperature. The primer may be, in some embodiments, single-stranded for maximum efficiency in amplification but may alternatively be double-stranded. If double-stranded, the primer is first treated to separate its strands before being used to prepare extension products. In some embodiments, the primer is an oligodeoxyribonucleotide. The primer must be sufficiently long to prime the synthesis of extension products. The exact lengths of the primers will depend on many factors, including temperature, source of primer, the polymerase enzyme (for example, whether it is thermostable), and use of the method.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

As used herein, the terms “library”, “RNA library” or “DNA library” or “library of DNA molecules” are used in accordance with their plain ordinary meaning and refer to a collection or a population of similarly sized nucleic acid fragments with known adapter sequences (e.g., known adapters attached to the 5′ and 3′ ends of each of the fragments). In embodiments, the library includes a plurality of nucleic acid fragments including one or more adapter sequences. In embodiments, the library includes circular nucleic acid templates. Libraries are typically prepared from input RNA, DNA, or cDNA and are processed by fragmentation, size selection, end-repair, adapter ligation, amplification, and purification. Alternative amplification-free (i.e., PCR free) methods for preparing a library of molecules include shearing input polynucleotides, size selecting and ligating adapters. A library may correspond to a single sample or a single origin. Multiple libraries, each with their own unique adapter sequences, may be pooled and sequenced in the same sequencing run using the methods described herein.

As used herein, the term “extension” or “elongation” is used in accordance with their plain and ordinary meanings and refer to synthesis by a polymerase of a new polynucleotide strand (e.g., an “extension strand”) complementary to a template strand by adding free nucleotides (e.g., dNTPs) from a reaction mixture that are complementary to the template in a 5′-to-3′ direction, including condensing a 5′-phosphate group of a dNTPs with a 3′-hydroxy group at the end of the nascent (elongating) DNA strand.

As used herein, the terms “sequencing”, “sequence determination”, “determining a nucleotide sequence”, and the like include determination of a partial or complete sequence information, including the identification, ordering, or locations of the nucleotides that comprise the polynucleotide being sequenced, and inclusive of the physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein comprises contacting a template and an annealed primer with a suitable polymerase under conditions suitable for polymerase extension and/or sequencing.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

As used herein, the term “hybridize” or “specifically hybridize” refers to a process where two complementary nucleic acid strands anneal to each other under appropriately stringent conditions. Hybridizations are typically and preferably conducted with oligonucleotides. The terms “annealing” and “hybridization” are used interchangeably to mean the formation of a stable duplex. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). Those skilled in the art understand how to estimate and adjust the stringency of hybridization conditions such that sequences having at least a desired level of complementarity will stably hybridize, while those having lower complementarity will not. As used herein, hybridization of a primer, or of a DNA extension product, respectively, is extendable by creation of a phosphodiester bond with an available nucleotide or nucleotide analogue capable of forming a phosphodiester bond, therewith.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

As used herein, the term “library”, when used in reference to nucleic acids, is intended to mean a collection of nucleic acids having different chemical compositions (e.g., different sequence, different length, etc.). Typically, the nucleic acids in a library will be different species having a common feature or characteristic of a genus or class, but otherwise differing in some way. For example, a library can include nucleic acid species that differ in nucleotide sequence, but that are similar with respect to having a sugar-phosphate backbone. A library can be created using techniques known in the art. Nucleic acids exemplified herein can include nucleic acids obtained from any source, including for example, digestion of a genome (e.g., a human genome) or a mixture of genomes. In another example, nucleic acids can be those obtained from metagenomic studies of a particular environment or ecosystem. The term also includes artificially created nucleic acid libraries such as DNA libraries.

The terms “click chemistry” and “click reaction” are used interchangeably herein and are intended to be consistent with their use in the art. Generally, click chemistry reactions are fast (e.g., quick to completion of reaction), simple, easily purified, and regiospecific. Click chemistry includes reactions such as, but not limited to, copper catalyzed azide-alkyne cycloaddition (CuAAC); strain-promoted azide-alkyne cycloaddition (SPAAC) also known as copper-free click chemistry; strain-promoted alkyne-nitrone cycloaddition (SPANC); alkyne hydrothiolation; and alkene hydrothiolation. Click chemistry using copper as a catalyst often includes a Cu(I) stabilizing ligand that is labile. Without being bound by any particular theory, the ligand can stabilize or protect the Cu(I) ion from oxidizing from the reactive Cu(I) to Cu(II) and can also act as a proton acceptor reducing or eliminating requirement of a base in the reaction. Click chemistry between polynucleotides can in some embodiments, be assisted by using a moiety that brings the two reacting partners in close enough proximity to react.

As used herein, and unless otherwise specified, the term “azide” or “azido” refers to N3, or —N═N+═N—, or —N—N+°N.

As used herein, “labels” are chemical or biochemical moieties useful for labeling a nucleic acid. “Labels” include, for example, fluorescent agents, chemiluminescent agents, affinity agents, blocking groups, chromogenic agents, quenching agents, radionucleotides, enzymes, substrates, cofactors, inhibitors, nanoparticles, magnetic particles, and other moieties known in the art. Labels are capable of generating a measurable signal and may be covalently or non-covalently joined to an oligonucleotide or nucleotide. In some examples, an oligonucleotide or a portion of an oligonucleotide of oligonucleotide-tethered nucleotide as disclosed herein may serve as a label.

As used herein, the term “tagged adapter-target polynucleotide” refers to an adapter-target polynucleotide that has one or more tags bound to it.

As used herein, the term “tag” refers to a biomolecule capable of binding an adapter-target polynucleotide. In embodiments, the tag includes a polynucleotide sequence which is complementary to a region of the target polynucleotide. In embodiments, the tag is capable of binding to the complementary region of the target polynucleotide. In embodiments, the tag includes one half of a cognate binding pair.

As used herein, the term “solid support” refers to an insoluble, chemically inert object that has a solid surface. Examples of a solid support provided herein include, but are not limited to, a bead, a column, a chip, a well, an array, a microfluidic channel, a resin, a particle, a microparticle, or a nanoparticle. In embodiments, the surface of the solid support can have a smooth, a porous, or a granular surface. In embodiments, the solid support includes one half of a cognate pair. In embodiments, the surface of the solid support includes one half of a cognate pair.

As used herein, the term “cognate binding pair” refers to a pair of biomolecules which non-covalently bind each other. In embodiments, the cognate binding pair comprises a capture moiety and a binding moiety. In some embodiments, the solid support comprises a capture moiety. In some embodiments, the tag comprises a binding moiety. In embodiments, the non-covalent binding of the cognate pair forms a cognate capture complex. In some embodiments, the non-covalent binding of the cognate pair forms a pulldown complex. In embodiments, the cognate pair includes the binding pairs of streptavidin and biotin, maltose and maltose binding protein, glutathione and glutathione S-transferase, chitin and chitin binding protein, an aptamer and its antigen, SpyCatcher and SpyTag, or an antibody and its antigen. In embodiments, the cognate pair includes the binding pair of streptavidin and biotin. In embodiments, the cognate pair includes the binding pair of maltose and maltose binding protein. In embodiments, the cognate pair includes the binding pair of glutathione and glutathione S-transferase. In embodiments, the cognate pair includes the binding pair of chitin and chitin binding protein. In embodiments, the cognate pair includes the binding pair of an aptamer and its antigen. In embodiments, the cognate pair includes the binding pair of SpyCatcher and SpyTag. In embodiments, the cognate pair includes the binding pair of an antibody and its antigen.

As used herein, the term “pulldown complex” refers to the complex formed when a binding moiety hybridizes to its corresponding capture moiety, thereby binding a tagged adapter-target polynucleotide to a solid support. In embodiments, the pulldown complex includes a tagged adapter-target polynucleotide and a solid support.

As used herein, the term “sequencing adapter” refers to a synthetic oligonucleotide or synthetic polynucleotide that flanks either side or flanks both sides of a polynucleotide sequence that will be sequenced. In embodiments, the sequencing adapter includes a flow cell binding sequence. In embodiments, the sequencing adapter includes a sequencer primer binding site. In embodiments, the sequencing adapter includes an index region or a barcode region. In embodiments, the sequencing adapter includes a motor protein binding site.

As used herein, the term “motor protein binding site” refers to an oligonucleotide sequence that allows for the binding of a motor protein. In embodiments, the motor protein is a polypeptide that unwinds double-stranded polynucleotides for sequencing of a single-stranded polynucleotide. In embodiments, the motor protein includes helicase activity.

As used herein, the term “flow cell binding sequence” refers to an oligonucleotide sequence that is complementary to the oligonucleotides attached to the sequencing flow cell. In embodiments, the flow cell binding sequence binds the polynucleotide sequence of interest to the flow cell.

As used herein, the term “sequencer primer binding site” refers to an oligonucleotide sequence allows for the binding of a sequencing primer. In embodiments, a sequencing primer is a synthetic oligonucleotide that recruits a polymerase to bind and extend the oligo synthesis.

As used herein, the term “index region” or “barcode region” refers to region of a sequencing adapter that allows for the individual sequencing adapter to be identified. In embodiments, one or more sequencing adapters include one or more index regions which allow for multiplexing.

As used herein, the term “sequencing complex” refers to a complex formed by the ligation of one or more sequencing adapters to an adapter-target polynucleotide. In embodiments, the sequencing complex includes one or more sequencing adapters and an adapter-target polynucleotide.

As used herein, the term “sequencing” refers to a process of determining the sequence of a biopolymer. In embodiments, the biopolymer is a polynucleotide. In embodiments, the sequencing does not include bisulfite sequencing. In embodiments, the sequencing does not include whole-genome sequencing.

As used herein, the term “bisulfite sequencing” refers to a sequencing method used to determine the methylation pattern of a polynucleotide. In embodiments, bisulfite sequencing includes bisulfite conversion. In embodiments, the bisulfite conversion includes treating a polynucleotide with bisulfite before sequencing. In embodiments, the bisulfite treatment introduces specific changes in the sequence of the polynucleotide. In embodiments, the bisulfite treatment converts cytosine residues to uracil. In embodiments, the bisulfite treatment does not convert 5-methylcytosine residues to uracil. In embodiments, the bisulfite sequencing includes reduced representation bisulfite sequencing (RRBS).

As used herein, the term “reduced representation bisulfite sequencing” or “RRBS” refers to a sequencing technique that analyzes the genome-wide methylation pattern on a single nucleotide level. In embodiments, RRBS includes restriction enzymes and bisulfite sequencing to enrich for areas of the genome with a high CpG content. In embodiments, RRBS does not target any promoter region.

As used herein, the term “reduced methylation sequencing” or “RRMS” refers to a sequencing technique that analyzes the genome-wide methylation pattern without bisulfite conversion. In embodiments, RRMS includes adaptive sampling to enrich for regions of interest. In embodiments, adaptive sampling deletes off-target regions during sequencing.

As used herein, the term “whole genome sequencing” or “WGS” refers to a sequencing technique used to determine the entirety or nearly the entirety of the nucleotide sequence of an organism's genome.

As used herein, the term “cell-free DNA” or “cfDNA” refers to freely circulating (e.g., not contained within a cell) DNA fragments found in a biological sample.

As used herein, the term “non-standard DNA nucleotide-targeting enzyme” refers generally to an enzyme capable of detecting and/or recognizing a non-standard DNA nucleotide and catalyzing the excision of the non-standard DNA nucleotide. In some embodiments, the non-standard DNA nucleotide-targeting enzyme is a uridine-targeting endonuclease.

As used herein, the term “uridine-targeting endonuclease” refers generally to an enzyme capable or detecting and/or recognizing a uridine nucleotide and catalyzing the excision of the uridine nucleotide. In some embodiments, the uridine-targeting endonuclease is a USER II endonuclease.

As used herein, the term “USER II endonuclease” refers to an enzyme that is capable of detecting and/or recognizing a uridine nucleotide and catalyzing the excision of the uridine nucleotide. In some embodiments, the USER II endonuclease includes a uracil DNA glycosylase (UDG) and an endonuclease III. The terms “USER II endonuclease” and “USER II enzyme” are used interchangeably herein.

Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, semen, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; horse; goat; pig; dog; cat; a rodent, e.g., guinea pig, rat, mouse; rabbit; or a bird; reptile; or fish. In embodiments the biological sample is obtained from a vertebrate animal. In embodiments, the biological sample is obtained from an invertebrate animal. In embodiments, the biological sample is obtained from a bacterium. In embodiments, the biological sample is obtained from a virus. In embodiments, the biological sample is obtained from a plant. In embodiments, the biologial sample is obtained from an archaca.

As used herein, the term “barcode” refers to a known nucleic acid sequence that allows some feature of a polynucleotide with which the barcode is associated to be identified. In some embodiments, the feature of the polynucleotide to be identified is the sample or source from which the polynucleotide is derived. In some embodiments, barcodes are at least 2, 3, 4, 5, 6, 7, 8, 9, 10, 11, 12, 13, 14, 15, or more nucleotides in length. In some embodiments, barcodes are shorter than 10, 9, 8, 7, 6, 5, or 4 nucleotides in length. In some embodiments, barcodes associated with some polynucleotides are of a different length than barcodes associated with other polynucleotides. In general, barcodes are of sufficient length and comprise sequences that are sufficiently different to allow the identification of samples based on barcodes with which they are associated. In some embodiments, each barcode in a plurality of barcodes differs from every other barcode in the plurality at two or more nucleotide positions, such as at 2, 3, 4, 5, 6, 7, 8, 9, 10, or more positions. In some embodiments, one or more adaptors comprise(s) at least one of a plurality of barcode sequences. In some embodiments, methods of the technology further comprise identifying the polynucleotide based on a barcode sequence to which the polynucleotide is joined. In general, a barcode may comprise an oligonucleotide sequence that when joined to a polynucleotide serves as an identifier of the sample from which the polynucleotide was derived.

### Methods

As already noted herein, there exists a need for improvement in sequencing methods. In particular, methods that can provide sequence information (e.g., sequence variance) while capturing various types of base modifications. Current methods can be limited, time consuming and/or unduly expensive. Provided herein are various improved methods that allow for targeted assessment of variation, epigenetic patterns and unique characteristics of a sequence that may not be captured or identified using existing technologies and methods. For example, the instant methods provide improvements for assessment of modified bases at specific regions of a sequence, e.g., at a particular genomic region, in a cost-effective and accurate manner.

The methods provided herein are, inter alia, useful for sequencing nucleic acids (e.g., target polynucleotides). The methods described herein including embodiments thereof are effective for identifying a base modification (e.g., methylation) in a nucleic acid. The sequencing methods provided herein may include targeting of specific sequences and/or regions within a genome. The targeting of specific sequences and/or regions allows the methods provided herein to utilize fewer resources (e.g., reagents, time, etc.) because the sequencing is targeted. In addition, the methods provided herein are capable of targeting specific sequences and/or regions within a genome to extend the life of a sequencer. Thus, provided herein are methods for sequencing target polynucleotides. In some embodiments, the target polynucleotide is enriched without amplification. In some embodiments, the sequencing includes detecting or identifying a base modification in the target polynucleotide.

In some embodiments, the method includes a step for ligating adapters to a target polynucleotide. In some embodiments, the step includes ligating one or more adapters to a target polynucleotide to form an adapter-target polynucleotide. In some embodiments, the one or more adapters include a primer binding site.

In some embodiments, the method includes a step for hybridizing tags to the target polynucleotide. In some embodiments, the step includes hybridizing one or more tags to the target polynucleotide to form a tagged adapter-target polynucleotide. In some embodiments, the one or more tags include a polynucleotide sequence complementary to the target polynucleotide. In some embodiments, the one or more tags include one half of a cognate pair. In some embodiments, the one or more tags include a binding moiety.

In some embodiments, the method optionally includes an isolation step prior to the adapter ligation step (e.g., step (a)). In embodiments, the isolation step includes contacting the target polynucleotide with one or more isolation probes prior to step (a) (e.g., the adapter ligation step). In some embodiments, the contacting of the target polynucleotide with one or more isolation probes occurs under conditions promoting hybridization of the one or more isolation probes to the target polynucleotide. In some embodiments, the hybridization of the one or more isolation probes to the target polynucleotide creates an isolation probe-target polynucleotide complex. In some embodiments, the one or more isolation probes are complementary to at least a portion of the target polynucleotide.

In some embodiments, the method optionally includes an isolation step after the adapter ligation step (e.g., step (a)) and prior to the second strand synthesis step (e.g., step (b)). In embodiments, the isolation step includes contacting the adapter-target polynucleotide with one or more isolation probes after step (a) (e.g., the adapter ligation step) and prior to step (b) (e.g., the second strand synthesis step). In some embodiments, the contacting of the adapter-target polynucleotide with one or more isolation probes occurs under conditions promoting hybridization of the one or more isolation probes to the adapter-target polynucleotide. In some embodiments, the hybridization of the one or more isolation probes to the adapter-target polynucleotide creates an isolation probe-adapter-target polynucleotide complex. In some embodiments, the one or more isolation probes are complementary to at least a portion of the adapter-target polynucleotide.

In some embodiments, the one or more isolation probes include a uridine nucleotide. In some embodiments, the one or more isolation probes do not include a thymidine nucleotide. In some embodiments, the one or more isolation probes include a first portion of a cognate binding pair.

In some embodiments, the isolation step optionally includes contacting the isolation probe-target polynucleotide complex with a solid support. In some embodiments, the isolation step optionally includes contacting the isolation probe-adapter-target polynucleotide complex with a solid support. In some embodiments, the solid support includes a second portion of a cognate binding pair. In some embodiments, the contacting of the isolation probe-target polynucleotide complex with a solid support occurs under conditions promoting non-covalent binding of the first portion of the cognate binding pair with the second portion of the cognate binding pair. In some embodiments, the contacting of the isolation probe-adapter-target polynucleotide complex with a solid support occurs under conditions promoting non-covalent binding of the first portion of the cognate binding pair with the second portion of the cognate binding pair. In some embodiments, the non-covalent binding of the first portion and the second portion of the cognate binding pair creates a pulldown complex. In some embodiments, the isolation step optionally includes isolating the pulldown complex.

In some embodiments, the isolation step optionally includes contacting the pulldown complex with a nonstandard DNA nucleotide-targeting enzyme. In some embodiments, the contacting of the pulldown complex with the nonstandard DNA nucleotide-targeting enzyme results in cleavage of nonstandard DNA nucleotides. In some embodiments, the nonstandard DNA nucleotide-targeting enzyme cleaves the one or more isolation probes. In some embodiments, the cleavage of the one or more isolation probes creates a plurality of cleaved isolation probes. In some embodiments, the plurality of cleaved isolation probes is degraded, denatured, eluted, or removed from the target polynucleotide. In some embodiments, the plurality of cleaved isolation probes is degraded, denatured, eluted, or removed from the adapter-target polynucleotide. In some embodiments, the plurality of cleaved isolation probes is degraded. In some embodiments, the plurality of cleaved isolation probes is denatured from the target polynucleotide. In some embodiments, the plurality of cleaved isolation probes is denatured from the adapter-target polynucleotide. In some embodiments, the plurality of cleaved isolation probes is eluted from the target polynucleotide. In some embodiments, the plurality of cleaved isolation probes is eluted from the adapter-target polynucleotide. In some embodiments, the plurality of cleaved isolation probes is removed from the target polynucleotide. In some embodiments, the plurality of cleaved isolation probes is removed from the adapter-target polynucleotide.

In some embodiments, the non-standard DNA nucleotide-targeting enzyme is a uridine-targeting endonuclease. In some embodiments, the uridine-targeting endonuclease is capable of excising uridine nucleotides in the one or more isolation probes. In some embodiments, uridine-targeting endonuclease is a USER II enzyme.

In some embodiments, the one or more isolation probes are one or more RNA-based isolation probes. In some embodiments, the one or more isolation probes are RNA. In some embodiments, the non-standard DNA-targeting enzyme is a ribonuclease (RNase). In some embodiments, the RNase cleaves the one or more RNA isolation probes. In some embodiments, the cleavage of the one or more RNA isolation probes creates a plurality of cleaved RNA isolation probes or cleaved RNA isolation probe fragments. In some embodiments, the plurality of cleaved RNA isolation probes is degraded, denatured, eluted, or removed from the target polynucleotide. In some embodiments, the plurality of cleaved RNA isolation probes is degraded, denatured, eluted, or removed from the adapter-target polynucleotide. In some embodiments, adapter-target polynucleotide is eluted from the pulldown complex with an RNase.

In some embodiments, the method optionally further includes an elution step to elute the target polynucleotide from the pulldown complex. In some embodiments, the method optionally further includes an elution step to elute the adapter-target polynucleotide from the pulldown complex. In some embodiments, the optional elution step includes increasing the pH of the buffer. In some embodiments, the increasing of the pH includes adding a base to the buffer. In some embodiments, the increasing of the pH includes adding NaOH to the buffer. In some embodiments, the optional elution step includes increasing the temperature of the buffer. In some embodiments, the increased temperature denatures the one or more isolation probes from the target polynucleotide. In some embodiments, the increased temperature denatures the one or more isolation probes from the adapter-target polynucleotide.

In some embodiments, the method optionally includes a step including formation of a pulldown complex. In some embodiments, the formation of the pulldown complex includes the binding of a cognate pair (e.g., cognate binding pair). In some embodiments, the formation of the pulldown complex includes the binding of a first portion of a cognate binding pair to a second portion of a cognate binding pair. In some embodiments, the formation of the pulldown complex includes the binding of a binding moiety to a capture moiety. In some embodiments, the pulldown complex includes the tagged adapter-target polynucleotide bound to a solid support. In some embodiments, the solid support includes one half of a cognate pair. In some embodiments, the solid support includes a capture moiety.

In some embodiments, the method optionally includes a step for pulldown the target polynucleotide. In some embodiments, the pulldown step includes isolating the pulldown complex. In some embodiments, the isolation of the pulldown complex enriches the target polynucleotide. In embodiments, the isolation of the pulldown complex may include exposing the pulldown complex to a magnet. In embodiments, the isolation of the pulldown complex may include exposing the pulldown complex to a magnet for about 1 minute. In embodiments, the isolation of the pulldown complex may include one or more exposures to a magnet. In embodiments, the isolation of the pulldown complex may include one or more exposures of the pulldown complex to a magnet, wherein each exposure is about 1 minute. In embodiments, the isolation of the pulldown complex may include two exposures of the pulldown complex to a magnet, wherein each exposure is about 1 minute. In embodiments, the isolation of the pulldown complex may include three exposures of the pulldown complex to a magnet, wherein each exposure is about 1 minute. In embodiments, the isolation of the pulldown complex may include incubating the pulldown complex with one or more wash buffers. In embodiments, the isolation of the pulldown complex may include incubating the pulldown complex with one wash buffer. In embodiments, the isolation of the pulldown complex may include incubating the pulldown complex with two wash buffers. In embodiments, the pulldown complex is incubated with a wash buffer for 5 minutes. In embodiments, the pulldown complex is incubated with one or more wash buffers, wherein each incubation occurs for 5 minutes. In embodiments, the pulldown complex is incubated with a wash buffer at 68° C. In embodiments, the pulldown complex is incubated with a wash buffer at 48° C.

In some embodiments, the method optionally includes a step for eluting the target polynucleotide from the pulldown complex. In some embodiments, the optional step for eluting the target polynucleotide includes eluting the adapter-target polynucleotide from the pulldown complex. In embodiments, the elution of the adapter-target polynucleotide from the pulldown complex may include contacting the pulldown complex with a buffer, e.g. an elution buffer. In embodiments, the elution of the adapter-target polynucleotide from the pulldown complex may include incubation (e.g., with the buffer) for about 5 minutes. In embodiments, the elution of the adapter-target polynucleotide from the pulldown complex may include incubation at room temperature (RT). In embodiments, the buffer may include an RNase. In embodiments, the RNase is RNase H. For example, when the adapter (probe) comprises RNA, addition of RNase to the buffer during elution allows removal of the adapter and elution in the same step.

In some embodiments, the method includes a step for second strand synthesis. In some embodiments, the step includes synthesizing a complementary adapter-target polynucleotide. In some embodiments, the pulldown complex or eluted adapter-target polynucleotide is contacted with a polymerase under conditions that promote creation of a complementary adapter-target polynucleotide. In some embodiments, the complementary adapter-target polynucleotide includes a 3′ protective group. In some embodiments, the 3′ protective group prevents sequencing of the synthesized complementary adapter-target polynucleotide. In some embodiments, the synthesized complementary adapter-target polynucleotide includes a 5′ protective group. In some embodiments, the 5′ protective group prevents sequencing of the synthesized complementary adapter-target polynucleotide.

In some embodiments, the second strand synthesis includes a barcode primer, a homopolymer primer, or a random hexamer primer.

In some embodiments, the second strand synthesis includes a barcode primer. In some embodiments, the second strand synthesis includes: (i) contacting the target polynucleotide with a DNA end-repair composition to produce an end-repaired target polynucleotide; (ii) contacting the end-repaired target polynucleotide with a ligase and one or more barcode sequences to produce a barcoded target polynucleotide; (iii) contacting the barcoded target polynucleotide with one or more isolation probes under conditions promoting hybridization of the one or more isolation probes to the barcoded target polynucleotide to produce an isolation probe-barcoded target polynucleotide complex, wherein the isolation probes include a first portion of a cognate binding pair and wherein the one or more isolation probes are complementary to at least a portion of the barcoded target polynucleotide; (iv) contacting the isolation probe-barcoded target polynucleotide complex with a solid support comprising a second portion of the cognate binding pair under conditions promoting non-covalent binding of the first portion of the cognate binding pair to the second portion of the cognate binding pair to produce a pulldown complex; (v) isolating the pulldown complex; (vi) denaturing the isolation probes to elute the barcoded target polynucleotide from the pulldown complex; (vii) contacting the isolated and eluted barcoded target polynucleotide with a barcode primer, wherein the barcode primer is a nucleic acid that is complementary to the barcode in the presence of a polymerase under conditions promoting extension of the barcode primer to produce a complementary barcoded target polynucleotide. In some embodiments, the barcode primer includes a 5′ protective group. In some embodiments, the 5′ protective group prevents sequencing of the complementary barcoded target polynucleotide (e.g., synthetic strand).

In some embodiments, the second strand synthesis includes a homopolymer primer. In some embodiments, the second strand synthesis includes: (i) denaturing the target polynucleotide; (ii) contacting the denatured target polynucleotide with a transferase to add a homopolymer tail to the target polynucleotide, thereby producing a homopolymer tailed-target polynucleotide; (iii) contacting the homopolymer tailed-target polynucleotide with one or more isolation probes under conditions promoting hybridization of the one or more isolation probes to the homopolymer tailed-target polynucleotide to produce an isolation probe-homopolymer tailed-target polynucleotide complex, wherein the isolation probes include a first portion of a cognate binding pair and wherein the one or more isolation probes are complementary to at least a portion of the homopolymer tailed-target polynucleotide; (iv) contacting the isolation probe-homopolymer tailed-target polynucleotide complex with a solid support comprising a second portion of the cognate binding pair under conditions promoting non-covalent binding of the first portion of the cognate binding pair to the second portion of the cognate binding pair to produce a pulldown complex; (v) isolating the pulldown complex; (vi) denaturing the isolation probes to elute the homopolymer tailed-target polynucleotide from the pulldown complex; (vii) contacting the isolated and eluted homopolymer tailed-target polynucleotide with a homopolymer primer, wherein the homopolymer primer is a nucleic acid that is complementary to the homopolymer tail added to the target polynucleotide in the presence of a polymerase under conditions promoting extension of the homopolymer primer to produce a complementary homopolymer tailed-target polynucleotide. In some embodiments, the homopolymer primer includes a 5′ protective group. In some embodiments, the 5′ protective group prevents sequencing of the complementary homopolymer tailed-target polynucleotide (e.g., synthetic strand).

In some embodiments, the transferase is a terminal deoxynucleotidyl transferase (TdT). In some embodiments, the homopolymer tail includes one or more of the same nucleotide. In some embodiments, the homopolymer tail is a polyadenine (poly-A) tail, a polycytosine (poly-C) tail, a polyguanine (poly-G) tail, or a polythymine (poly-T) tail. In some embodiments, the homopolymer tail is a polyadenine (poly-A) tail. In some embodiments, the homopolymer tail is a polycytosine (poly-C) tail. In some embodiments, the homopolymer tail is a polyguanine (poly-G) tail. In some embodiments, the homopolymer tail is a polythymine (poly-T) tail. In some embodiments, the homopolymer tail is added to the 5′ end or the 3′ end of the target polynucleotide. In some embodiments, the homopolymer tail is added to the 5′ end of the target polynucleotide. In some embodiments, the homopolymer tail is added to the 3′ end of the target polynucleotide. In some embodiments, the second strand synthesis includes contacting the complementary homopolymer tailed-target polynucleotide with an exonuclease prior to sequencing to remove the homopolymer tail from the 3′ end of the complementary homopolymer tailed-target polynucleotide. In some embodiments the exonuclease is a 3′->5′ exonuclease. In some embodiments, the exonuclease is a Klenow exo-enzyme (e.g., Klenow fragment).

In some embodiments, the second strand synthesis includes a random hexamer primer. In some embodiments, the second strand synthesis includes: (i) denaturing the target polynucleotide to produce a denatured target polynucleotide; (ii) contacting the denatured target polynucleotide with one or more isolation probes under conditions promoting hybridization of the one or more isolation probes to the denatured target polynucleotide to produce an isolation probe-denatured target polynucleotide complex, wherein the isolation probes include a first portion of a cognate binding pair and wherein the one or more isolation probes are complementary to at least a portion of the denatured target polynucleotide; (iii) contacting the isolation probe-denatured target polynucleotide complex with a solid support comprising a second portion of the cognate binding pair under conditions promoting non-covalent binding of the first portion of the cognate binding pair to the second portion of the cognate binding pair to produce a pulldown complex; (iv) isolating the pulldown complex; (v) denaturing the isolation probes to elute the denatured target polynucleotide from the pulldown complex; (vi) contacting the isolated and eluted denatured target polynucleotide with one or more random hexamer primers, wherein the one or more random hexamer primers are nucleic acid sequences that is complementary to at least a portion of the denatured target polynucleotide in the presence of a polymerase under conditions promoting extension of the one or more random hexamer primer to produce a complementary denatured target polynucleotide. In some embodiments, step (vi) further includes a ligase to repair a nick and/or gap in the complementary denatured target polynucleotide. In some embodiments, the second strand synthesis optionally includes an end-repair step comprising contacting the denatured target polynucleotide and/or the complementary target polynucleotide to produce an end-repaired target polynucleotide and/or an end-repaired complementary target polynucleotide.

In some embodiments, the method includes a step for formation of a sequencing complex. In some embodiments, the step includes ligating a sequencing adapter to the adapter-target polynucleotide to form a sequencing complex. In some embodiments, the 3′ protective group prevents ligation of the sequencing adapter to the complementary adapter-target polynucleotide. In some embodiments, the 5′ protective group prevents ligation of the sequencing adapter to the complementary adapter-target polynucleotide. In some embodiments, a primer without a 5′ is used to prevent ligation of the sequencing adapter to the complementary adapter-target polynucleotide.

In some embodiments, the method includes a step including sequencing the sequencing complex. In some embodiments, the complementary adapter-target polynucleotide is not sequenced.

In some embodiments, the method steps described herein may be optional. In some embodiments, the method steps described herein may be excluded from the method.

In an aspect is provided a method for sequencing a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with a first polymerase under conditions promoting creation of a complementary adapter-target polynucleotide, wherein the complementary adapter-target polynucleotide includes a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing includes sequencing of the target polynucleotide sequence without sequencing the complementary adapter-target polynucleotide, thereby sequencing the target polynucleotide, optionally wherein the target polynucleotide includes one or more base modifications.

In another aspect is provided a method for detecting a base modification in a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with a first polymerase under conditions promoting creation of a complementary adapter-target polynucleotide, wherein the complementary adapter-target polynucleotide includes a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing includes sequencing of the target polynucleotide sequence without sequencing the complementary adapter-target polynucleotide, thereby detecting the base modification in the target polynucleotide.

In another aspect is provided a method for quantifying a base modification in a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with a first polymerase under conditions promoting creation of a complementary adapter-target polynucleotide, wherein the complementary adapter-target polynucleotide includes a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing includes sequencing of the target polynucleotide sequence without sequencing the complementary adapter-target polynucleotide, thereby quantifying the base modification in the target polynucleotide.

In another aspect is provided a method for sequencing a plurality of target polynucleotides, the method including: (a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters include a primer binding site; (b) contacting the plurality of adapter-target polynucleotides with a first polymerase under conditions promoting creation of a plurality of complementary adapter-target polynucleotides, wherein each of the complementary adapter-target polynucleotides includes a 3′ protective group or a 5′ protective group; (c) contacting the plurality of adapter-target polynucleotides with a sequencing adapter in the presence of a second ligase under conditions promoting ligation of the sequencing adapter to the adapter-target polynucleotide to create a plurality of sequencing complexes; and (d) sequencing the plurality of sequencing complexes, wherein the sequencing includes sequencing of the plurality of the target polynucleotide sequences without sequencing the plurality of complementary adapter-target polynucleotides, thereby sequencing the plurality of target polynucleotides, optionally wherein the plurality of target polynucleotides includes one or more base modifications.

In another aspect is provided a method for detecting a plurality of base modifications in a plurality of target polynucleotides, the method including: (a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters include a primer binding site; (b) contacting the plurality of adapter-target polynucleotides with a first polymerase under conditions promoting creation of a plurality of complementary adapter-target polynucleotides, wherein each of the complementary adapter-target polynucleotides includes a 3′ protective group or a 5′ protective group; (c) contacting the plurality of adapter-target polynucleotides with a sequencing adapter in the presence of a second ligase under conditions promoting ligation of the sequencing adapter to the adapter-target polynucleotide to create a plurality of sequencing complexes; and (d) sequencing the plurality of sequencing complexes, wherein the sequencing includes sequencing of the plurality of the target polynucleotide sequences without sequencing the plurality of complementary adapter-target polynucleotides, thereby detecting the plurality of base modifications in the plurality of target polynucleotides.

In another aspect is provided a method for detecting a plurality of base modifications in a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with a first polymerase under conditions promoting creation of a complementary adapter-target polynucleotide, wherein the complementary adapter-target polynucleotide includes a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing includes sequencing of the target polynucleotide sequence without sequencing the complementary adapter-target polynucleotide, thereby detecting the plurality of base modifications in the target polynucleotide.

In another aspect is provided a method for detecting a base modification in a plurality of target polynucleotides, the method including: (a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters include a primer binding site; (b) contacting the plurality of adapter-target polynucleotides with a first polymerase under conditions promoting creation of a plurality of complementary adapter-target polynucleotides, wherein each of the complementary adapter-target polynucleotides includes a 3′ protective group or a 5′ protective group; (c) contacting the plurality of adapter-target polynucleotides with a sequencing adapter in the presence of a second ligase under conditions promoting ligation of the sequencing adapter to the adapter-target polynucleotide to create a plurality of sequencing complexes; and (d) sequencing the plurality of sequencing complexes, wherein the sequencing includes sequencing of the plurality of the target polynucleotide sequences without sequencing the plurality of complementary adapter-target polynucleotides, thereby detecting the base modification in the plurality of target polynucleotides.

In another aspect is provided a method for sequencing a target polynucleotide, the method including: (a) dephosphorylating the target polynucleotide; (b) contacting the target polynucleotide with one or more adapters in the presence of a first ligase under conditions promoting ligation of the one or more adapters to the target polynucleotide to create an adapter-target polynucleotide, wherein the one or more adapters optionally include a primer binding site, a first portion of a cognate binding pair, and/or a uridine; (c) contacting the adapter-target polynucleotide with a solid support including a second portion of a cognate binding pair under conditions allowing the binding of the first portion of a cognate binding pair and a second portion of a cognate binding pair to create a pulldown complex; (d) contacting the adapter-target polynucleotide with a first polymerase under conditions promoting creation of a complementary adapter-target polynucleotide, wherein the complementary adapter-target polynucleotide includes a 3′ protective group or a 5′ protective group; (e) optionally contacting the adapter-target polynucleotide and complementary adapter-target polynucleotide with one or more barcodes or ligation adapters in the presence of a second ligase under conditions promoting ligation of the one or more barcodes or ligation adapters to the adapter-target polynucleotide and complementary adapter-target polynucleotide; (f) optionally the pulldown complex is isolated; (g) the pulldown complex is contacted with a uridine-targeted enzyme to excise a uridine residue and elute the adapter-target polynucleotide and complementary adapter-target polynucleotide from the solid support, thereby forming a sequencing complex; and (h) sequencing the sequencing complex, wherein the sequencing includes sequencing of the adapter-target polynucleotide sequence without sequencing the complementary adapter-target polynucleotide, thereby sequencing the target polynucleotide, optionally wherein the target polynucleotide includes one or more base modifications.

In another aspect is provided a method for enriching a target polynucleotide.

In another aspect is provided a method of preparing a library of a plurality of target polynucleotides.

In some embodiments, the method further includes contacting the adapter-target polynucleotides after step (b) with a solid support including a second portion of a cognate binding pair, under conditions promoting hybridization of the first portion of a cognate binding pair to the second portion of a cognate binding pair, to create a pulldown complex; isolating the pulldown complex; and cleaving the adapter-target polynucleotide to remove the cognate binding pair and solid support before step (c).

In some embodiments, the first polymerase is a Q5U polymerase.

In some embodiments, step (b) includes a deoxyuridine triphosphate. In embodiments, step (b) does not include a deoxythymidine triphosphate. In some embodiments, step (b) includes a deoxyuridine triphosphate and does not include a deoxythymidine triphosphate.

In some embodiments, the complementary adapter-target polynucleotide includes a uridine and does not include a thymidine.

In some embodiments, the complementary adapter-target polynucleotide is contacted with a uridine-targeting enzyme, wherein the uridine-targeting enzyme excises a uridine residue.

In some embodiments, step (b) includes a DNA polymerase I and an E. coli ligase.

In embodiments, the conditions promoting ligation of the one or more adapters to the target polynucleotide to create an adapter-target polynucleotide include the presence of a first ligase, one or more adapters, and a target polynucleotide in an appropriate buffer and at a suitable temperature. In embodiments, the buffer includes appropriate components (e.g., pH, ionic strength, cofactors, etc.) that facilitate ligation of the one or more adapters to the target polynucleotide. In embodiments, the conditions promoting ligation of the one or more adapters to the target polynucleotide to create an adapter-target polynucleotide may include one or more adapters. In embodiments, the conditions promoting ligation of the one or more adapters to the target polynucleotide to create an adapter-target polynucleotide may include one or more barcodes. In embodiments, the conditions promoting ligation of the one or more adapters to the target polynucleotide to create an adapter-target polynucleotide may include a ligation enhancer. In embodiments, the conditions promoting ligation of the one or more adapters to the target polynucleotide to create an adapter-target polynucleotide may include a first ligase. In embodiments, the conditions promoting ligation of the one or more adapters to the target polynucleotide to create an adapter-target polynucleotide may include T4 DNA ligase. In embodiments, the conditions promoting ligation of the one or more adapters to the target polynucleotide to create an adapter-target polynucleotide may include polyethylene glycol (PEG). In embodiments, the conditions promoting ligation of the one or more adapters to the target polynucleotide to create an adapter-target polynucleotide may include adenosine triphosphate (ATP). In embodiments, the conditions promoting ligation of the one or more adapters to the target polynucleotide to create an adapter-target polynucleotide may include incubation at room temperature (RT). In embodiments, the conditions promoting ligation of the one or more adapters to the target polynucleotide to create an adapter-target polynucleotide may include incubation for about 15 minutes.

In embodiments, the conditions promoting ligation of the one or more adapters to the target polynucleotide to create an adapter-target polynucleotide may include incubation from about 16° C. to about 37° C. In embodiments, the conditions promoting ligation of the one or more adapters to the target polynucleotide to create an adapter-target polynucleotide may include incubation from about 17° C. to about 37° C. In embodiments, the conditions promoting ligation of the one or more adapters to the target polynucleotide to create an adapter-target polynucleotide may include incubation from about 18° C. to about 37° C. In embodiments, the conditions promoting ligation of the one or more adapters to the target polynucleotide to create an adapter-target polynucleotide may include incubation from about 19° C. to about 37° C. In embodiments, the conditions promoting ligation of the one or more adapters to the target polynucleotide to create an adapter-target polynucleotide may include incubation from about 20° C. to about 37° C. In embodiments, the conditions promoting ligation of the one or more adapters to the target polynucleotide to create an adapter-target polynucleotide may include incubation from about 21° C. to about 37° C. In embodiments, the conditions promoting ligation of the one or more adapters to the target polynucleotide to create an adapter-target polynucleotide may include incubation from about 22° C. to about 37° C. In embodiments, the conditions promoting ligation of the one or more adapters to the target polynucleotide to create an adapter-target polynucleotide may include incubation from about 23° C. to about 37° C. In embodiments, the conditions promoting ligation of the one or more adapters to the target polynucleotide to create an adapter-target polynucleotide may include incubation from about 24° C. to about 37° C. In embodiments, the conditions promoting ligation of the one or more adapters to the target polynucleotide to create an adapter-target polynucleotide may include incubation from about 25° C. to about 37° C. In embodiments, the conditions promoting ligation of the one or more adapters to the target polynucleotide to create an adapter-target polynucleotide may include incubation from about 26° C. to about 37° C. In embodiments, the conditions promoting ligation of the one or more adapters to the target polynucleotide to create an adapter-target polynucleotide may include incubation from about 27° C. to about 37° C. In embodiments, the conditions promoting ligation of the one or more adapters to the target polynucleotide to create an adapter-target polynucleotide may include incubation from about 28° C. to about 37° C. In embodiments, the conditions promoting ligation of the one or more adapters to the target polynucleotide to create an adapter-target polynucleotide may include incubation from about 29° C. to about 37° C. In embodiments, the conditions promoting ligation of the one or more adapters to the target polynucleotide to create an adapter-target polynucleotide may include incubation from about 30° C. to about 37° C. In embodiments, the conditions promoting ligation of the one or more adapters to the target polynucleotide to create an adapter-target polynucleotide may include incubation from about 31° C. to about 37° C. In embodiments, the conditions promoting ligation of the one or more adapters to the target polynucleotide to create an adapter-target polynucleotide may include incubation from about 32° C. to about 37° C. In embodiments, the conditions promoting ligation of the one or more adapters to the target polynucleotide to create an adapter-target polynucleotide may include incubation from about 33° C. to about 37° C. In embodiments, the conditions promoting ligation of the one or more adapters to the target polynucleotide to create an adapter-target polynucleotide may include incubation from about 34° C. to about 37° C. In embodiments, the conditions promoting ligation of the one or more adapters to the target polynucleotide to create an adapter-target polynucleotide may include incubation from about 35° C. to about 37° C. In embodiments, the conditions promoting ligation of the one or more adapters to the target polynucleotide to create an adapter-target polynucleotide may include incubation from about 36° C. to about 37° C.

In embodiments, the conditions promoting ligation of the one or more adapters to the target polynucleotide to create an adapter-target polynucleotide may include incubation from about 16° C. to about 36° C. In embodiments, the conditions promoting ligation of the one or more adapters to the target polynucleotide to create an adapter-target polynucleotide may include incubation from about 16° C. to about 35° C. In embodiments, the conditions promoting ligation of the one or more adapters to the target polynucleotide to create an adapter-target polynucleotide may include incubation from about 16° C. to about 34° C. In embodiments, the conditions promoting ligation of the one or more adapters to the target polynucleotide to create an adapter-target polynucleotide may include incubation from about 16° C. to about 33° C. In embodiments, the conditions promoting ligation of the one or more adapters to the target polynucleotide to create an adapter-target polynucleotide may include incubation from about 16° C. to about 32° C. In embodiments, the conditions promoting ligation of the one or more adapters to the target polynucleotide to create an adapter-target polynucleotide may include incubation from about 16° C. to about 31° C. In embodiments, the conditions promoting ligation of the one or more adapters to the target polynucleotide to create an adapter-target polynucleotide may include incubation from about 16° C. to about 30° C. In embodiments, the conditions promoting ligation of the one or more adapters to the target polynucleotide to create an adapter-target polynucleotide may include incubation from about 16° C. to about 29° C. In embodiments, the conditions promoting ligation of the one or more adapters to the target polynucleotide to create an adapter-target polynucleotide may include incubation from about 16° C. to about 28° C. In embodiments, the conditions promoting ligation of the one or more adapters to the target polynucleotide to create an adapter-target polynucleotide may include incubation from about 16° C. to about 27° C. In embodiments, the conditions promoting ligation of the one or more adapters to the target polynucleotide to create an adapter-target polynucleotide may include incubation from about 16° C. to about 26° C. In embodiments, the conditions promoting ligation of the one or more adapters to the target polynucleotide to create an adapter-target polynucleotide may include incubation from about 16° C. to about 25° C. In embodiments, the conditions promoting ligation of the one or more adapters to the target polynucleotide to create an adapter-target polynucleotide may include incubation from about 16° C. to about 24° C. In embodiments, the conditions promoting ligation of the one or more adapters to the target polynucleotide to create an adapter-target polynucleotide may include incubation from about 16° C. to about 23° C. In embodiments, the conditions promoting ligation of the one or more adapters to the target polynucleotide to create an adapter-target polynucleotide may include incubation from about 16° C. to about 22° C. In embodiments, the conditions promoting ligation of the one or more adapters to the target polynucleotide to create an adapter-target polynucleotide may include incubation from about 16° C. to about 21° C. In embodiments, the conditions promoting ligation of the one or more adapters to the target polynucleotide to create an adapter-target polynucleotide may include incubation from about 16° C. to about 20° C. In embodiments, the conditions promoting ligation of the one or more adapters to the target polynucleotide to create an adapter-target polynucleotide may include incubation from about 16° C. to about 19° C. In embodiments, the conditions promoting ligation of the one or more adapters to the target polynucleotide to create an adapter-target polynucleotide may include incubation from about 16° C. to about 18° C. In embodiments, the conditions promoting ligation of the one or more adapters to the target polynucleotide to create an adapter-target polynucleotide may include incubation from about 17° C. to about 36° C.

In embodiments, the conditions promoting hybridization of the one or more tags to the adapter-target polynucleotide to create a tagged adapter-target polynucleotide include the presence of one or more tags and an adapter-target polynucleotide in an appropriate buffer and at a suitable temperature. In embodiments, the buffer includes appropriate components (e.g., pH, ionic strength, cofactors, etc.) that facilitate hybridization of the one or more tags to the adapter-target polynucleotide. In embodiments, the conditions promoting hybridization of the one or more tags to the adapter-target polynucleotide to create a tagged adapter-target polynucleotide may include a hybridization enhancer. In embodiments, the conditions promoting hybridization of the one or more tags to the adapter-target polynucleotide to create a tagged adapter-target polynucleotide may include a synthetic polynucleotide. In embodiments, the synthetic polynucleotide prevents nonspecific binding. In embodiments, the conditions promoting hybridization of the one or more tags to the adapter-target polynucleotide to create a tagged adapter-target polynucleotide may include a nonspecific synthetic polynucleotide. In embodiments, the nonspecific synthetic polynucleotide prevents nonspecific binding. In embodiments, the conditions promoting hybridization of the one or more tags to the adapter-target polynucleotide to create a tagged adapter-target polynucleotide may include DNA from a different species than the target polynucleotide. In embodiments, the conditions promoting hybridization of the one or more tags to the adapter-target polynucleotide to create a tagged adapter-target polynucleotide may include salmon sperm DNA. In embodiments the salmon sperm DNA prevents nonspecific binding. In embodiments, the conditions promoting hybridization of the one or more tags to the adapter-target polynucleotide to create a tagged adapter-target polynucleotide may include incubation at about 70° C. In embodiments, the conditions promoting hybridization of the one or more tags to the adapter-target polynucleotide to create a tagged adapter-target polynucleotide may include incubation for about 20 hours.

In embodiments, the conditions promoting hybridization of the one or more tags to the adapter-target polynucleotide to create a tagged adapter-target polynucleotide may include incubation from about 50° C. to about 80° C. In embodiments, the conditions promoting hybridization of the one or more tags to the adapter-target polynucleotide to create a tagged adapter-target polynucleotide may include incubation from about 55° C. to about 80° C. In embodiments, the conditions promoting hybridization of the one or more tags to the adapter-target polynucleotide to create a tagged adapter-target polynucleotide may include incubation from about 60° C. to about 80° C. In embodiments, the conditions promoting hybridization of the one or more tags to the adapter-target polynucleotide to create a tagged adapter-target polynucleotide may include incubation from about 65° C. to about 80° C. In embodiments, the conditions promoting hybridization of the one or more tags to the adapter-target polynucleotide to create a tagged adapter-target polynucleotide may include incubation from about 70° C. to about 80° C. In embodiments, the conditions promoting hybridization of the one or more tags to the adapter-target polynucleotide to create a tagged adapter-target polynucleotide may include incubation from about 75° C. to about 80° C.

In embodiments, the conditions promoting hybridization of the one or more tags to the adapter-target polynucleotide to create a tagged adapter-target polynucleotide may include incubation from about 50° C. to about 75° C. In embodiments, the conditions promoting hybridization of the one or more tags to the adapter-target polynucleotide to create a tagged adapter-target polynucleotide may include incubation from about 50° C. to about 70° C. In embodiments, the conditions promoting hybridization of the one or more tags to the adapter-target polynucleotide to create a tagged adapter-target polynucleotide may include incubation from about 50° C. to about 65° C. In embodiments, the conditions promoting hybridization of the one or more tags to the adapter-target polynucleotide to create a tagged adapter-target polynucleotide may include incubation from about 50° C. to about 60° C. In embodiments, the conditions promoting hybridization of the one or more tags to the adapter-target polynucleotide to create a tagged adapter-target polynucleotide may include incubation from about 50° C. to about 55° C.

In embodiments, the conditions promoting hybridization of the one or more tags to the solid support to create a pulldown complex include the presence of one or more tags bound to an adapter-target polynucleotide and a solid support in an appropriate buffer and at a suitable temperature. In embodiments, the buffer includes appropriate components (e.g., pH, ionic strength, cofactors, etc.) that facilitate hybridization of the one or more tags to the solid support. In embodiments, the conditions promoting hybridization of the one or more tags to the solid support to create a pulldown complex may include incubation at about 68° C. In embodiments, the conditions promoting hybridization of the one or more tags to the solid support to create a pulldown complex may include incubation for about 5 minutes.

In embodiments, the conditions promoting creation of a complementary adapter-target polynucleotide include the presence of nucleotide triphosphates and a polymerase enzyme (e.g., a thermostable polymerase enzyme) in an appropriate buffer and at a suitable temperature. In embodiments, the buffer includes appropriate components (e.g., pH, ionic strength, cofactors, etc.) that facilitate nucleic acid synthesis. In embodiments, the conditions promoting creation of a complementary adapter-target polynucleotide may include standard polymerase chain reaction conditions. In embodiments, the conditions promoting creation of a complementary adapter-target polynucleotide may include incubation at about 60° C. In embodiments, the conditions promoting creation of a complementary adapter-target polynucleotide may include incubation for about 15 minutes. In embodiments, the conditions promoting creation of a complementary adapter-target polynucleotide may include polyethylene glycol (PEG). In embodiments, the conditions promoting creation of a complementary adapter-target polynucleotide may include adenosine triphosphate (ATP). In embodiments, the conditions promoting creation of a complementary adapter-target polynucleotide may include a Bst 3.0 DNA polymerase. In embodiments, the conditions promoting creation of a complementary adapter-target polynucleotide may include a BST buffer. In embodiments, the conditions promoting creation of a complementary adapter-target polynucleotide may include an elution primer. In embodiments, the conditions promoting creation of a complementary adapter-target polynucleotide may include one or more free nucleotides. In embodiments, the conditions promoting creation of a complementary adapter-target polynucleotide may include one or more deoxynucleotide triphosphates. In embodiments, the conditions promoting creation of a complementary adapter-target polynucleotide may include MgSO₄.

In embodiments, the conditions promoting ligation of the sequencing adapter to the adapter-target polynucleotide to create a sequencing complex include the presence of a second ligase, a sequencing adapter, and an adapter-target polynucleotide in an appropriate buffer and at a suitable temperature. In embodiments, the buffer includes appropriate components (e.g., pH, ionic strength, cofactors, etc.) that facilitate ligation of the sequencing adapter to the adapter-target polynucleotide. In embodiments, the conditions promoting ligation of the sequencing adapter to the adapter-target polynucleotide to create a sequencing complex may include incubation for about 15 minutes. In embodiments, the conditions promoting ligation of the sequencing adapter to the adapter-target polynucleotide to create a sequencing complex may include incubation at room temperature (RT). In embodiments, the conditions promoting ligation of the sequencing adapter to the adapter-target polynucleotide to create a sequencing complex may include a ligation adapter (LA). In embodiments, the conditions promoting ligation of the sequencing adapter to the adapter-target polynucleotide to create a sequencing complex may include a ligation buffer (LNB). In embodiments, the conditions promoting ligation of the sequencing adapter to the adapter-target polynucleotide to create a sequencing complex may include a Quick Ligase. In embodiments, the conditions promoting ligation of the sequencing adapter to the adapter-target polynucleotide to create a sequencing complex may include a Quick T4 Ligase.

In embodiments, the conditions promoting ligation of the sequencing adapter to the adapter-target polynucleotide to create a sequencing complex may include incubation from about 16° C. to about 37° C. In embodiments, the conditions promoting ligation of the sequencing adapter to the adapter-target polynucleotide to create a sequencing complex may include incubation from about 17° C. to about 37° C. In embodiments, the conditions promoting ligation of the sequencing adapter to the adapter-target polynucleotide to create a sequencing complex may include incubation from about 18° C. to about 37° C. In embodiments, the conditions promoting ligation of the sequencing adapter to the adapter-target polynucleotide to create a sequencing complex may include incubation from about 19° C. to about 37° C. In embodiments, the conditions promoting ligation of the sequencing adapter to the adapter-target polynucleotide to create a sequencing complex may include incubation from about 20° C. to about 37° C. In embodiments, the conditions promoting ligation of the sequencing adapter to the adapter-target polynucleotide to create a sequencing complex may include incubation from about 21° C. to about 37° C. In embodiments, the conditions promoting ligation of the sequencing adapter to the adapter-target polynucleotide to create a sequencing complex may include incubation from about 22° C. to about 37° C. In embodiments, the conditions promoting ligation of the sequencing adapter to the adapter-target polynucleotide to create a sequencing complex may include incubation from about 23° C. to about 37° C. In embodiments, the conditions promoting ligation of the sequencing adapter to the adapter-target polynucleotide to create a sequencing complex may include incubation from about 24° C. to about 37° C. In embodiments, the conditions promoting ligation of the sequencing adapter to the adapter-target polynucleotide to create a sequencing complex may include incubation from about 25° C. to about 37° C. In embodiments, the conditions promoting ligation of the sequencing adapter to the adapter-target polynucleotide to create a sequencing complex may include incubation from about 26° C. to about 37° C. In embodiments, the conditions promoting ligation of the sequencing adapter to the adapter-target polynucleotide to create a sequencing complex may include incubation from about 27° C. to about 37° C. In embodiments, the conditions promoting ligation of the sequencing adapter to the adapter-target polynucleotide to create a sequencing complex may include incubation from about 28° C. to about 37° C. In embodiments, the conditions promoting ligation of the sequencing adapter to the adapter-target polynucleotide to create a sequencing complex may include incubation from about 29° C. to about 37° C. In embodiments, the conditions promoting ligation of the sequencing adapter to the adapter-target polynucleotide to create a sequencing complex may include incubation from about 30° C. to about 37° C. In embodiments, the conditions promoting ligation of the sequencing adapter to the adapter-target polynucleotide to create a sequencing complex may include incubation from about 31° C. to about 37° C. In embodiments, the conditions promoting ligation of the sequencing adapter to the adapter-target polynucleotide to create a sequencing complex may include incubation from about 32° C. to about 37° C. In embodiments, the conditions promoting ligation of the sequencing adapter to the adapter-target polynucleotide to create a sequencing complex may include incubation from about 33° C. to about 37° C. In embodiments, the conditions promoting ligation of the sequencing adapter to the adapter-target polynucleotide to create a sequencing complex may include incubation from about 34° C. to about 37° C. In embodiments, the conditions promoting ligation of the sequencing adapter to the adapter-target polynucleotide to create a sequencing complex may include incubation from about 35° C. to about 37° C. In embodiments, the conditions promoting ligation of the sequencing adapter to the adapter-target polynucleotide to create a sequencing complex may include incubation from about 36° C. to about 37° C.

In embodiments, the conditions promoting ligation of the sequencing adapter to the adapter-target polynucleotide to create a sequencing complex may include incubation from about 16° C. to about 36° C. In embodiments, the conditions promoting ligation of the sequencing adapter to the adapter-target polynucleotide to create a sequencing complex may include incubation from about 16° C. to about 35° C. In embodiments, the conditions promoting ligation of the sequencing adapter to the adapter-target polynucleotide to create a sequencing complex may include incubation from about 16° C. to about 34° C. In embodiments, the conditions promoting ligation of the sequencing adapter to the adapter-target polynucleotide to create a sequencing complex may include incubation from about 16° C. to about 33° C. In polynucleotide to create a sequencing complex may include incubation from about 16° C. to about 32° C. In embodiments, the conditions promoting ligation of the sequencing adapter to the adapter-target polynucleotide to create a sequencing complex may include incubation from about 16° C. to about 31° C. In embodiments, the conditions promoting ligation of the sequencing adapter to the adapter-target polynucleotide to create a sequencing complex may include incubation from about 16° C. to about 30° C. In embodiments, the conditions promoting ligation of the sequencing adapter to the adapter-target polynucleotide to create a sequencing complex may include incubation from about 16° C. to about 29° C. In embodiments, the conditions promoting ligation of the sequencing adapter to the adapter-target polynucleotide to create a sequencing complex may include incubation from about 16° C. to about 28° C. In embodiments, the conditions promoting ligation of the sequencing adapter to the adapter-target polynucleotide to create a sequencing complex may include incubation from about 16° C. to about 27° C. In embodiments, the conditions promoting ligation of the sequencing adapter to the adapter-target polynucleotide to create a sequencing complex may include incubation from about 16° C. to about 26° C. In embodiments, the conditions promoting ligation of the sequencing adapter to the adapter-target polynucleotide to create a sequencing complex may include incubation from about 16° C. to about 25° C. In embodiments, the conditions promoting ligation of the sequencing adapter to the adapter-target polynucleotide to create a sequencing complex may include incubation from about 16° C. to about 24° C. In embodiments, the conditions promoting ligation of the sequencing adapter to the adapter-target polynucleotide to create a sequencing complex may include incubation from about 16° C. to about 23° C. In embodiments, the conditions promoting ligation of the sequencing adapter to the adapter-target polynucleotide to create a sequencing complex may include incubation from about 16° C. to about 22° C. In embodiments, the conditions promoting ligation of the sequencing adapter to the adapter-target polynucleotide to create a sequencing complex may include incubation from about 16° C. to about 21° C. In embodiments, the conditions promoting ligation of the sequencing adapter to the adapter-target polynucleotide to create a sequencing complex may include incubation from about 16° C. to about 20° C. In embodiments, the conditions promoting ligation of the sequencing adapter to the adapter-target polynucleotide to create a sequencing complex may include incubation from about 16° C. to about 19° C. In embodiments, the conditions promoting ligation of the sequencing adapter to the adapter-target polynucleotide to create a sequencing complex may include incubation from about 16° C. to about 18° C. In polynucleotide to create a sequencing complex may include incubation from about 16° C. to about 17° C.

In some embodiments, the method further includes: (e) analyzing the sequenced sequencing complex to detect the base modification.

In some embodiments, the method further includes repeating steps (a) through (c) at least once with one or more additional adapters.

In some embodiments, the target polynucleotide is isolated from a biological sample prior to step (a). In embodiments, the biological sample is from a human.

In some embodiments, the target polynucleotide includes genomic DNA (gDNA) or cell-free DNA (cfDNA). In some embodiments, the target polynucleotide includes genomic DNA (gDNA). In some embodiments, the target polynucleotide includes cell-free DNA (cfDNA).

In some embodiments, the biological sample is denatured prior to step (a) (e.g., adapter ligation step). In some embodiments, the biological sample is denatured prior to the isolation step. In some embodiments, the gDNA is denatured prior to step (a) (e.g., adapter ligation step). In some embodiments, the gDNA is denatured prior to the isolation step. In some embodiments, the cfDNA is denatured prior to step (a) (e.g., adapter ligation step). In some embodiments, the cfDNA is denatured prior to the isolation step. In some embodiments, the target polynucleotide is denatured prior to step (a) (e.g., adapter ligation step). In some embodiments, the target polynucleotide is denatured prior to the isolation step.

In some embodiments, the target polynucleotide is fragmented prior to step (a). In some embodiments, the fragmenting includes mechanical fragmentation or enzymatic fragmentation. In some embodiments, the fragmenting includes mechanical fragmentation. In some embodiments, the mechanical fragmentation includes focused acoustic shearing, hydrodynamic shearing, or nebulization. In some embodiments, the mechanical fragmentation includes focused acoustic shearing. In some embodiments, the mechanical fragmentation includes hydrodynamic shearing. In some embodiments, the mechanical fragmentation includes nebulization. In some embodiments, the fragmenting includes enzymatic fragmentation. In some embodiments, the enzymatic fragmentation includes transposases, restriction enzymes, or non-specific nicking enzymes. In some embodiments, the enzymatic fragmentation includes transposases. In some embodiments, the enzymatic fragmentation includes restriction enzymes. In some embodiments, the enzymatic fragmentation includes non-specific nicking enzymes. In some embodiments, the target polynucleotide is fragmented into one or more target polynucleotide fragments.

In some embodiments, the target polynucleotide fragments are between about 75 base pairs (bp) and about 50,000 bp. In some embodiments, the target polynucleotide fragments are between about 100 bp and about 50,000 bp. In some embodiments, the target polynucleotide fragments are between about 125 bp and about 50,000 bp. In some embodiments, the target polynucleotide fragments are between about 150 bp and about 50,000 bp. In some embodiments, the target polynucleotide fragments are between about 175 bp and about 50,000 bp. In some embodiments, the target polynucleotide fragments are between about 200 bp and about 50,000 bp. In some embodiments, the target polynucleotide fragments are between about 300 bp and about 50,000 bp. In some embodiments, the target polynucleotide fragments are between about 400 bp and about 50,000 bp. In some embodiments, the target polynucleotide fragments are between about 500 bp and about 50,000 bp. In some embodiments, the target polynucleotide fragments are between about 600 bp and about 50,000 bp. In some embodiments, the target polynucleotide fragments are between about 700 bp and about 50,000 bp. In some embodiments, the target polynucleotide fragments are between about 800 bp and about 50,000 bp. In some embodiments, the target polynucleotide fragments are between about 900 bp and about 50,000 bp. In some embodiments, the target polynucleotide fragments are between about 1000 bp and about 50,000 bp. In some embodiments, the target polynucleotide fragments are between about 2000 bp and about 50,000 bp. In some embodiments, the target polynucleotide fragments are between about 3000 bp and about 50,000 bp. In some embodiments, the target polynucleotide fragments are between about 4000 bp and about 50,000 bp. In some embodiments, the target polynucleotide fragments are between about 5000 bp and about 50,000 bp. In some embodiments, the target polynucleotide fragments are between about 6000 bp and about 50,000 bp. In some embodiments, the target polynucleotide fragments are between about 7000 bp and about 50,000 bp. In some embodiments, the target polynucleotide fragments are between about 8000 bp and about 50,000 bp. In some embodiments, the target polynucleotide fragments are between about 9000 bp and about 50,000 bp. In some embodiments, the target polynucleotide fragments are between about 10,000 bp and about 50,000 bp. In some embodiments, the target polynucleotide fragments are between about 15,000 bp and about 50,000 bp. In some embodiments, the target polynucleotide fragments are between about 20,000 bp and about 50,000 bp. In some embodiments, the target polynucleotide fragments are between about 25,000 bp and about 50,000 bp. In some embodiments, the target polynucleotide fragments are between about 30,000 bp and about 50,000 bp. In some embodiments, the target polynucleotide fragments are between about 35,000 bp and about 50,000 bp. In some embodiments, the target polynucleotide fragments are between about 40,000 bp and about 50,000 bp. In some embodiments, the target polynucleotide fragments are between about 45,000 bp and about 50,000 bp.

In some embodiments, the target polynucleotide fragments are between about 75 bp and about 45,000 bp. In some embodiments, the target polynucleotide fragments are between about 75 bp and about 40,000 bp. In some embodiments, the target polynucleotide fragments are between about 75 bp and about 35,000 bp. In some embodiments, the target polynucleotide fragments are between about 75 bp and about 30,000 bp. In some embodiments, the target polynucleotide fragments are between about 75 bp and about 25,000 bp. In some embodiments, the target polynucleotide fragments are between about 75 bp and about 20,000 bp. In some embodiments, the target polynucleotide fragments are between about 75 bp and about 15,000 bp. In some embodiments, the target polynucleotide fragments are between about 75 bp and about 10,000 bp. In some embodiments, the target polynucleotide fragments are between about 75 bp and about 9,000 bp. In some embodiments, the target polynucleotide fragments are between about 75 bp and about 8,000 bp. In some embodiments, the target polynucleotide fragments are between about 75 bp and about 7,000 bp. In some embodiments, the target polynucleotide fragments are between about 75 bp and about 6,000 bp. In some embodiments, the target polynucleotide fragments are between about 75 bp and about 5,000 bp. In some embodiments, the target polynucleotide fragments are between about 75 bp and about 4,000 bp. In some embodiments, the target polynucleotide fragments are between about 75 bp and about 3,000 bp. In some embodiments, the target polynucleotide fragments are between about 75 bp and about 2,000 bp. In some embodiments, the target polynucleotide fragments are between about 75 bp and about 1,000 bp. In some embodiments, the target polynucleotide fragments are between about 75 bp and about 900 bp. In some embodiments, the target polynucleotide fragments are between about 75 bp and about 800 bp. In some embodiments, the target polynucleotide fragments are between about 75 bp and about 700 bp. In some embodiments, the target polynucleotide fragments are between about 75 bp and about 600 bp. In some embodiments, the target polynucleotide fragments are between about 75 bp and about 500 bp. In some embodiments, the target polynucleotide fragments are between about 75 bp and about 400 bp. In some embodiments, the target polynucleotide fragments are between about 75 bp and about 300 bp. In some embodiments, the target polynucleotide fragments are between about 75 bp and about 200 bp. In some embodiments, the target polynucleotide fragments are between about 75 bp and about 175 bp. In some embodiments, the target polynucleotide fragments are between about 75 bp and about 150 bp. In some embodiments, the target polynucleotide fragments are between about 75 bp and about 125 bp. In some embodiments, the target polynucleotide fragments are between about 75 bp and about 100 bp.

In some embodiments, the target polynucleotide fragments are between 75 base bp and 50,000 bp. In some embodiments, the target polynucleotide fragments are between 100 bp and 50,000 bp. In some embodiments, the target polynucleotide fragments are between 125 bp and 50,000 bp. In some embodiments, the target polynucleotide fragments are between 150 bp and 50,000 bp. In some embodiments, the target polynucleotide fragments are between 175 bp and 50,000 bp. In some embodiments, the target polynucleotide fragments are between 200 bp and 50,000 bp. In some embodiments, the target polynucleotide fragments are between 300 bp and 50,000 bp. In some embodiments, the target polynucleotide fragments are between 400 bp and 50,000 bp. In some embodiments, the target polynucleotide fragments are between 500 bp and 50,000 bp. In some embodiments, the target polynucleotide fragments are between 600 bp and 50,000 bp. In some embodiments, the target polynucleotide fragments are between 700 bp and 50,000 bp. In some embodiments, the target polynucleotide fragments are between 800 bp and 50,000 bp. In some embodiments, the target polynucleotide fragments are between 900 bp and 50,000 bp. In some embodiments, the target polynucleotide fragments are between 1000 bp and 50,000 bp. In some embodiments, the target polynucleotide fragments are between 2000 bp and 50,000 bp. In some embodiments, the target polynucleotide fragments are between 3000 bp and 50,000 bp. In some embodiments, the target polynucleotide fragments are between 4000 bp and 50,000 bp. In some embodiments, the target polynucleotide fragments are between 5000 bp and 50,000 bp. In some embodiments, the target polynucleotide fragments are between 6000 bp and 50,000 bp. In some embodiments, the target polynucleotide fragments are between 7000 bp and 50,000 bp. In some embodiments, the target polynucleotide fragments are between 8000 bp and 50,000 bp. In some embodiments, the target polynucleotide fragments are between 9000 bp and 50,000 bp. In some embodiments, the target polynucleotide fragments are between 10,000 bp and 50,000 bp. In some embodiments, the target polynucleotide fragments are between 15,000 bp and 50,000 bp. In some embodiments, the target polynucleotide fragments are between 20,000 bp and 50,000 bp. In some embodiments, the target polynucleotide fragments are between 25,000 bp and 50,000 bp. In some embodiments, the target polynucleotide fragments are between 30,000 bp and 50,000 bp. In some embodiments, the target polynucleotide fragments are between 35,000 bp and 50,000 bp. In some embodiments, the target polynucleotide fragments are between 40,000 bp and 50,000 bp. In some embodiments, the target polynucleotide fragments are between 45,000 bp and 50,000 bp.

In some embodiments, the target polynucleotide fragments are between 75 bp and 45,000 bp. In some embodiments, the target polynucleotide fragments are between 75 bp and 40,000 bp. In some embodiments, the target polynucleotide fragments are between 75 bp and 35,000 bp. In some embodiments, the target polynucleotide fragments are between 75 bp and 30,000 bp. In some embodiments, the target polynucleotide fragments are between 75 bp and 25,000 bp. In some embodiments, the target polynucleotide fragments are between 75 bp and 20,000 bp. In some embodiments, the target polynucleotide fragments are between 75 bp and 15,000 bp. In some embodiments, the target polynucleotide fragments are between 75 bp and 10,000 bp. In some embodiments, the target polynucleotide fragments are between 75 bp and 9,000 bp. In some embodiments, the target polynucleotide fragments are between 75 bp and 8,000 bp. In some embodiments, the target polynucleotide fragments are between 75 bp and 7,000 bp. In some embodiments, the target polynucleotide fragments are between 75 bp and 6,000 bp. In some embodiments, the target polynucleotide fragments are between 75 bp and 5,000 bp. In some embodiments, the target polynucleotide fragments are between 75 bp and 4,000 bp. In some embodiments, the target polynucleotide fragments are between 75 bp and 3,000 bp. In some embodiments, the target polynucleotide fragments are between 75 bp and 2,000 bp. In some embodiments, the target polynucleotide fragments are between 75 bp and 1,000 bp. In some embodiments, the target polynucleotide fragments are between 75 bp and 900 bp. In some embodiments, the target polynucleotide fragments are between 75 bp and 800 bp. In some embodiments, the target polynucleotide fragments are between 75 bp and 700 bp. In some embodiments, the target polynucleotide fragments are between 75 bp and 600 bp. In some embodiments, the target polynucleotide fragments are between 75 bp and 500 bp. In some embodiments, the target polynucleotide fragments are between 75 bp and 400 bp. In some embodiments, the target polynucleotide fragments are between 75 bp and 300 bp. In some embodiments, the target polynucleotide fragments are between 75 bp and 200 bp. In some embodiments, the target polynucleotide fragments are between 75 bp and 175 bp. In some embodiments, the target polynucleotide fragments are between 75 bp and 150 bp. In some embodiments, the target polynucleotide fragments are between 75 bp and 125 bp. In some embodiments, the target polynucleotide fragments are between 75 bp and 100 bp.

In some embodiments, the target polynucleotide is contacted with a DNA end-repair composition prior to step (a) (e.g., adapter ligation step). In some embodiments, the target polynucleotide is contacted with a DNA end-repair composition denatured prior to the isolation step. In some embodiments, the one or more target polynucleotide fragments are contacted with a DNA end-repair composition prior to step (a) (e.g., adapter ligation step). In some embodiments, the one or more target polynucleotide fragments are contacted with a DNA end-repair composition denatured prior to the isolation step.

In some embodiments, the one or more adapters further include a first barcode.

In some embodiments, the one or more additional adapters further include one or more additional barcodes.

In some embodiments, the one or more adapters include one half of a cognate pair. In some embodiments, the one or more adapters include one portion of a cognate binding pair. In some embodiments, the one or more adapters include one or more binding moieties. In some embodiments, the one or more adapters include streptavidin, biotin, maltose, maltose binding protein, glutathione, glutathione S-transferase, chitin, chitin binding protein, an aptamer, an antigen, SpyCatcher, SpyTag, or an antibody. In some embodiments, the one or more adapters include streptavidin. In some embodiments, the one or more adapters include biotin. In some embodiments, the one or more adapters include maltose. In some embodiments, the one or more adapters include maltose binding protein. In some embodiments, the one or more adapters include glutathione. In some embodiments, the one or more adapters include glutathione S-transferase. In some embodiments, the one or more adapters include chitin. In some embodiments, the one or more adapters include chitin binding protein. In some embodiments, the one or more adapters include an aptamer. In some embodiments, the one or more adapters include an antigen. In some embodiments, the one or more adapters include SpyCatcher. In some embodiments, the one or more adapters include SpyTag. In some embodiments, the one or more adapters include an antibody.

In some embodiments, the one or more adapters target the same region of the target polynucleotide.

In some embodiments, the one or more adapters target different regions of the target polynucleotide.

In some embodiments, the one or more adapters include an oligonucleotide. In some embodiments, the one or more adapters include an oligonucleotide sequence complementary to the target polynucleotide. In some embodiments, the one or more adapters are capable of binding to the target polynucleotide.

In some embodiments, the solid support includes one half of a cognate pair. In some embodiments, the solid support includes a capture moiety. In some embodiments, the solid support includes streptavidin, biotin, maltose, maltose binding protein, glutathione, glutathione S-transferase, chitin, chitin binding protein, an aptamer, an antigen, SpyCatcher, SpyTag, or an antibody. In some embodiments, the solid support includes streptavidin. In some embodiments, the solid support includes biotin. In some embodiments, the solid support includes maltose. In some embodiments, the solid support includes maltose binding protein. In some embodiments, the solid support includes glutathione. In some embodiments, the solid support includes glutathione S-transferase. In some embodiments, the solid support includes chitin. In some embodiments, the solid support includes chitin binding protein. In some embodiments, the solid support includes an aptamer. In some embodiments, the solid support includes an antigen. In some embodiments, the solid support includes SpyCatcher. In some embodiments, the solid support includes SpyTag. In some embodiments, the solid support includes an antibody.

In some embodiments, the solid support is a bead or a column. In some embodiments, the solid support is a bead. In some embodiments, the solid support is a column. In some embodiments, the bead is paramagnetic.

In some embodiments, the elution includes separating the adapter-target polynucleotide from the one or more adapters, thereby separating the target polynucleotide from the solid support. In some embodiments, the separating of the elution includes denaturing the one or more adapters. In some embodiments, the denaturing of the one or more adapters causes the one or more adapters to unzip, thereby separating the adapter from the target polynucleotide and/or the solid support. In some embodiments, the separating of the elution includes a strand-displacing polymerase displacing the one or more adapters from the target polynucleotide, thereby separating the target polynucleotide from the solid support and/or adapter. In some embodiments, the separating of the elution includes a polymerase with exonuclease activity digesting the one or more adapters, thereby separating the target polynucleotide from the solid support and/or the adapter.

In some embodiments, the elution includes NaOH, heat, a strand-displacing polymerase, a polymerase with exonuclease activity, or a uridine-targeting enzyme. In some embodiments, the elution includes a base. In embodiments, the elution may include incubation with a base for about 5 minutes. In embodiments, the elution may include incubation with a base at room temperature (RT). In some embodiments, the elution includes NaOH. In embodiments, the elution may include incubation with NaOH for about 5 minutes. In embodiments, the elution may include incubation with NaOH at room temperature (RT). In some embodiments, the elution includes heat. In some embodiments, the elution includes a strand-displacing polymerase. In some embodiments, the elution includes a polymerase with exonuclease activity. In some embodiments, the elution includes a uridine-targeting enzyme. In some embodiments, the uridine-targeting enzyme excises a uridine residue. In some embodiments, the uridine-targeting enzyme is a uracil-specific excision reagent (USER) enzyme. In some embodiments, the uracil-specific excision reagent (USER) includes a uracil DNA glycosylase (UDG). In some embodiments, the uracil-specific excision reagent (USER) includes a DNA glycosylase-lyase endonuclease VIII. In some embodiments the uracil-specific excision reagent (USER) includes a uracil DNA glycosylase (UDG) and a DNA glycosylase-lyase endonuclease VIII. In embodiments, the uracil-specific excision reagent (USER) is a thermolabile USER II enzyme. In embodiments, the uracil-specific excision reagent (USER) is a thermostable USER III enzyme.

In some embodiments, the 3′ protective group prevents sequencing of the complementary adapter-target polynucleotide. In some embodiments, the 3′ protective group includes a nucleotide overhang. In some embodiments, the nucleotide overhang includes at least one nucleotide, two nucleotides, or three nucleotides. In some embodiments, the nucleotide overhang includes at least one nucleotide. In some embodiments, the nucleotide overhang includes at least two nucleotides. In some embodiments, the nucleotide overhang includes at least three nucleotides.

In some embodiments, the 5′ protective group prevents sequencing of the complementary adapter-target polynucleotide. In some embodiments, the 5′ protective group includes an aldehyde. In some embodiments, the 5′ protective group includes an amine. In some embodiments, the 5′ protective group includes a thiol.

In some embodiments, the primer does not include a 5′ phosphate group.

In some embodiments, the sequencing adapter includes a second barcode.

In some embodiments, the base modification includes an n1-methyl-pseudouridine, a pseudouridine (Ψ), a 5-methylcytosine (5mC), a 5-hydroxymethylcytosine (5hmC), a 4-methylcytosine (4mC), an N 6-methyladenine (6 mA), an N 6-methyladenosine (m6a), an N 1-methyladenosine (m1aA), a 7-methylguanine (m7G), a 2′-O-methylation (2′-O-Methyl), a 5-bromo-2′-deoxyuridine (BrdU), or a 5-ethynyl-2′-deoxyuridine (ErdU). In some embodiments, the base modification includes an n1-methyl-pseudouridine. In some embodiments, the base modification includes a pseudouridine (Ψ). In some embodiments, the base modification includes a 5-methylcytosine (5mC). In some embodiments, the base modification includes a 5-hydroxymethylcytosine (5hmC). In some embodiments, the base modification includes a 4-methylcytosine (4mC). In some embodiments, the base modification includes an N 6-methyladenine (6 mA). In some embodiments, the base modification includes an N 6-methyladenosine (m6a). In some embodiments, the base modification includes an N 1-methyladenosine (m1aA). In some embodiments, the base modification includes a 7-methylguanine (m7G). In some embodiments, the base modification includes a 2′-O-methylation (2′-O-Methyl). In some embodiments, the base modification includes a 5-bromo-2′-deoxyuridine (BrdU). In some embodiments, the base modification includes a 5-ethynyl-2′-deoxyuridine (ErdU).

In some embodiments, the plurality of base modifications includes one or more of an n1-methyl-pseudouridine, a pseudouridine (Ψ), a 5-methylcytosine (5mC), a 5-hydroxymethylcytosine (5hmC), a 4-methylcytosine (4mC), an N 6-methyladenine (6 mA), an N 6-methyladenosine (m6a), an N 1-methyladenosine (m1aA), a 7-methylguanine (m7G), a 2′-O-methylation (2′-O-Methyl), a 5-bromo-2′-deoxyuridine (BrdU), or 5-ethynyl-2′-deoxyuridine (ErdU). In some embodiments, the plurality of base modifications includes an n1-methyl-pseudouridine. In some embodiments, the plurality of base modifications includes a pseudouridine (Ψ). In some embodiments, the plurality of base modifications includes a 5-methylcytosine (5mC). In some embodiments, the plurality of base modifications includes a 5-hydroxymethylcytosine (5hmC). In some embodiments, the base modification includes a 4-methylcytosine (4mC). In some embodiments, the plurality of base modifications includes a N 6-methyladenine (6 mA). In some embodiments, the plurality of base modifications includes an N 6-methyladenosine (m6a). In some embodiments, the plurality of base modifications includes an N 1-methyladenosine (m1aA). In some embodiments, the plurality of base modifications includes a 7-methylguanine (m7G). In some embodiments, the plurality of base modifications includes 2′-O-methylation (2′-O-Methyl). In some embodiments, the plurality of base modifications includes a 5-bromo-2′-deoxyuridine (BrdU). In some embodiments, the plurality of base modifications includes a 5-ethynyl-2′-deoxyuridine (ErdU).

In some embodiments, the method does not include bisulfite conversion or amplification of the target polynucleotide. In some embodiments, the method does not include bisulfite conversion. In some embodiments, the method does not include amplification of the target polynucleotide.

In some embodiments, the method does not include reduced representation bisulfite sequencing (RRBS) or reduced representation methylation sequencing (RRMS). In some embodiments, the method does not include reduced representation bisulfite sequencing (RRBS). In some embodiments, the method does not include reduced representation methylation sequencing (RRMS).

In some embodiments, the first ligase or the second ligase is a T4 DNA ligase, a Quick ligase, a T3 ligase, a T7 ligase, or a Tag ligase. In some embodiments, the first ligase or the second ligase is a T4 DNA ligase. In some embodiments, the first ligase or the second ligase is a Quick ligase. In some embodiments, the first ligase or the second ligase is a T3 ligase. In some embodiments, the first ligase or the second ligase is a T7 ligase. In some embodiments, the first ligase or the second ligase is a Tag ligase.

In some embodiments, the first ligase is a T4 DNA ligase, a Quick ligase, a T3 ligase, a T7 ligase, or a Tag ligase. In some embodiments, the first ligase is a T4 DNA ligase. In some embodiments, the first ligase is a Quick ligase. In some embodiments, the first ligase is a T3 ligase. In some embodiments, the first ligase is a T7 ligase. In some embodiments, the first ligase is a Tag ligase.

In some embodiments, the second ligase is a T4 DNA ligase, a Quick ligase, a T3 ligase, a T7 ligase, or a Tag ligase. In some embodiments, the second ligase is a T4 DNA ligase. In some embodiments, the second ligase is a Quick ligase. In some embodiments, the second ligase is a T3 ligase. In some embodiments, the second ligase is a T7 ligase. In some embodiments, the second ligase is a Tag ligase.

In some embodiments, the first polymerase is a Taq polymerase, a BST polymerase, a Sulfolobus polymerase, a Therminator polymerase, a Klenow polymerase, a deep vent polymerase. In some embodiments, the first polymerase is a Taq polymerase. In some embodiments, the first polymerase is a BST polymerase. In some embodiments, the first polymerase is a Sulfolobus polymerase. In some embodiments, the first polymerase is a Therminator polymerase. In some embodiments, the first polymerase is a Klenow polymerase. In some embodiments, the first polymerase is a deep vent polymerase.

In some embodiments, the methods described herein are performed in a buffer. In some embodiments, the method is performed in a buffer. In some embodiments, each step of the method are performed in a buffer. In some embodiments, the buffer includes water. In some embodiments, the buffer includes nuclease free water. In some embodiments, the buffer includes one or more of the following: a ligation buffer, a hybridization buffer, a blocking buffer, an extension buffer, a second strand synthesis buffer, a fragmentation buffer, an elution buffer, a sequencing buffer, and/or combinations thereof. In some embodiments, the buffer includes a ligation buffer In some embodiments, the buffer includes a hybridization buffer In some embodiments, the buffer includes a blocking buffer, an extension buffer, a second strand synthesis buffer In some embodiments, the buffer includes a fragmentation buffer In some embodiments, the buffer includes an elution buffer In some embodiments, the buffer includes a sequencing buffer. Buffers for use in sequencing or library preparation methods are well known in the art and are widely available commercially.

In some embodiments, the sequencing is performed on a sequencer capable of identifying modified bases.

In some embodiments, the base modification in the target polynucleotide is retained until sequencing.

In some embodiments, the method enriches the base modification in the target polynucleotide between about 300-fold to about 2400-fold compared to whole genome sequencing. In some embodiments, the method enriches the base modification in the target polynucleotide between about 400-fold to about 2400-fold compared to whole genome sequencing. In some embodiments, the method enriches the base modification in the target polynucleotide between about 500-fold to about 2400-fold compared to whole genome sequencing. In some embodiments, the method enriches the base modification in the target polynucleotide between about 600-fold to about 2400-fold compared to whole genome sequencing. In some embodiments, the method enriches the base modification in the target polynucleotide between about 700-fold to about 2400-fold compared to whole genome sequencing. In some embodiments, the method enriches the base modification in the target polynucleotide between about 800-fold to about 2400-fold compared to whole genome sequencing. In some embodiments, the method enriches the base modification in the target polynucleotide between about 900-fold to about 2400-fold compared to whole genome sequencing. In some embodiments, the method enriches the base modification in the target polynucleotide between about 1000-fold to about 2400-fold compared to whole genome sequencing. In some embodiments, the method enriches the base modification in the target polynucleotide between about 1100-fold to about 2400-fold compared to whole genome sequencing. In some embodiments, the method enriches the base modification in the target polynucleotide between about 1200-fold to about 2400-fold compared to whole genome sequencing. In some embodiments, the method enriches the base modification in the target polynucleotide between about 1300-fold to about 2400-fold compared to whole genome sequencing. In some embodiments, the method enriches the base modification in the target polynucleotide between about 1400-fold to about 2400-fold compared to whole genome sequencing. In some embodiments, the method enriches the base modification in the target polynucleotide between about 1500-fold to about 2400-fold compared to whole genome sequencing. In some embodiments, the method enriches the base modification in the target polynucleotide between about 1600-fold to about 2400-fold compared to whole genome sequencing. In some embodiments, the method enriches the base modification in the target polynucleotide between about 1700-fold to about 2400-fold compared to whole genome sequencing. In some embodiments, the method enriches the base modification in the target polynucleotide between about 1800-fold to about 2400-fold compared to whole genome sequencing. In some embodiments, the method enriches the base modification in the target polynucleotide between about 1900-fold to about 2400-fold compared to whole genome sequencing. In some embodiments, the method enriches the base modification in the target polynucleotide between about 2000-fold to about 2400-fold compared to whole genome sequencing. In some embodiments, the method enriches the base modification in the target polynucleotide between about 2100-fold to about 2400-fold compared to whole genome sequencing. In some embodiments, the method enriches the base modification in the target polynucleotide between about 2200-fold to about 2400-fold compared to whole genome sequencing. In some embodiments, the method enriches the base modification in the target polynucleotide between about 2300-fold to about 2400-fold compared to whole genome sequencing.

In some embodiments, the method enriches the base modification in the target polynucleotide between about 300-fold to about 2300-fold compared to whole genome sequencing. In some embodiments, the method enriches the base modification in the target polynucleotide between about 300-fold to about 2200-fold compared to whole genome sequencing. In some embodiments, the method enriches the base modification in the target polynucleotide between about 300-fold to about 2100-fold compared to whole genome sequencing. In some embodiments, the method enriches the base modification in the target polynucleotide between about 300-fold to about 2000-fold compared to whole genome sequencing. In some embodiments, the method enriches the base modification in the target polynucleotide between about 300-fold to about 1900-fold compared to whole genome sequencing. In some embodiments, the method enriches the base modification in the target polynucleotide between about 300-fold to about 1800-fold compared to whole genome sequencing. In some embodiments, the method enriches the base modification in the target polynucleotide between about 300-fold to about 1700-fold compared to whole genome sequencing. In some embodiments, the method enriches the base modification in the target polynucleotide between about 300-fold to about 1600-fold compared to whole genome sequencing. In some embodiments, the method enriches the base modification in the target polynucleotide between about 300-fold to about 1500-fold compared to whole genome sequencing. In some embodiments, the method enriches the base modification in the target polynucleotide between about 300-fold to about 1400-fold compared to whole genome sequencing. In some embodiments, the method enriches the base modification in the target polynucleotide between about 300-fold to about 1300-fold compared to whole genome sequencing. In some embodiments, the method enriches the base modification in the target polynucleotide between about 300-fold to about 1200-fold compared to whole genome sequencing. In some embodiments, the method enriches the base modification in the target polynucleotide between about 300-fold to about 1100-fold compared to whole genome sequencing. In some embodiments, the method enriches the base modification in the target polynucleotide between about 300-fold to about 1000-fold compared to whole genome sequencing. In some embodiments, the method enriches the base modification in the target polynucleotide between about 300-fold to about 900-fold compared to whole genome sequencing. In some embodiments, the method enriches the base modification in the target polynucleotide between about 300-fold to about 800-fold compared to whole genome sequencing. In some embodiments, the method enriches the base modification in the target polynucleotide between about 300-fold to about 700-fold compared to whole genome sequencing. In some embodiments, the method enriches the base modification in the target polynucleotide between about 300-fold to about 600-fold compared to whole genome sequencing. In some embodiments, the method enriches the base modification in the target polynucleotide between about 300-fold to about 500-fold compared to whole genome sequencing. In some embodiments, the method enriches the base modification in the target polynucleotide between about 300-fold to about 400-fold compared to whole genome sequencing.

In some embodiments, the method enriches the base modification in the target polynucleotide between 300-fold to 2400-fold compared to whole genome sequencing. In some embodiments, the method enriches the base modification in the target polynucleotide between 400-fold to 2400-fold compared to whole genome sequencing. In some embodiments, the method enriches the base modification in the target polynucleotide between 500-fold to 2400-fold compared to whole genome sequencing. In some embodiments, the method enriches the base modification in the target polynucleotide between 600-fold to 2400-fold compared to whole genome sequencing. In some embodiments, the method enriches the base modification in the target polynucleotide between 700-fold to 2400-fold compared to whole genome sequencing. In some embodiments, the method enriches the base modification in the target polynucleotide between 800-fold to 2400-fold compared to whole genome sequencing. In some embodiments, the method enriches the base modification in the target polynucleotide between 900-fold to 2400-fold compared to whole genome sequencing. In some embodiments, the method enriches the base modification in the target polynucleotide between 1000-fold to 2400-fold compared to whole genome sequencing. In some embodiments, the method enriches the base modification in the target polynucleotide between 1100-fold to 2400-fold compared to whole genome sequencing. In some embodiments, the method enriches the base modification in the target polynucleotide between 1200-fold to 2400-fold compared to whole genome sequencing. In some embodiments, the method enriches the base modification in the target polynucleotide between 1300-fold to 2400-fold compared to whole genome sequencing. In some embodiments, the method enriches the base modification in the target polynucleotide between 1400-fold to 2400-fold compared to whole genome sequencing. In some embodiments, the method enriches the base modification in the target polynucleotide between 1500-fold to 2400-fold compared to whole genome sequencing. In some embodiments, the method enriches the base modification in the target polynucleotide between 1600-fold to 2400-fold compared to whole genome sequencing. In some embodiments, the method enriches the base modification in the target polynucleotide between 1700-fold to 2400-fold compared to whole genome sequencing. In some embodiments, the method enriches the base modification in the target polynucleotide between 1800-fold to 2400-fold compared to whole genome sequencing. In some embodiments, the method enriches the base modification in the target polynucleotide between 1900-fold to 2400-fold compared to whole genome sequencing. In some embodiments, the method enriches the base modification in the target polynucleotide between 2000-fold to 2400-fold compared to whole genome sequencing. In some embodiments, the method enriches the base modification in the target polynucleotide between 2100-fold to 2400-fold compared to whole genome sequencing. In some embodiments, the method enriches the base modification in the target polynucleotide between 2200-fold to 2400-fold compared to whole genome sequencing. In some embodiments, the method enriches the base modification in the target polynucleotide between 2300-fold to 2400-fold compared to whole genome sequencing.

In some embodiments, the method enriches the base modification in the target polynucleotide between 300-fold to 2300-fold compared to whole genome sequencing. In some embodiments, the method enriches the base modification in the target polynucleotide between 300-fold to 2200-fold compared to whole genome sequencing. In some embodiments, the method enriches the base modification in the target polynucleotide between 300-fold to 2100-fold compared to whole genome sequencing. In some embodiments, the method enriches the base modification in the target polynucleotide between 300-fold to 2000-fold compared to whole genome sequencing. In some embodiments, the method enriches the base modification in the target polynucleotide between 300-fold to 1900-fold compared to whole genome sequencing. In some embodiments, the method enriches the base modification in the target polynucleotide between 300-fold to 1800-fold compared to whole genome sequencing. In some embodiments, the method enriches the base modification in the target polynucleotide between 300-fold to 1700-fold compared to whole genome sequencing. In some embodiments, the method enriches the base modification in the target polynucleotide between 300-fold to 1600-fold compared to whole genome sequencing. In some embodiments, the method enriches the base modification in the target polynucleotide between 300-fold to 1500-fold compared to whole genome sequencing. In some embodiments, the method enriches the base modification in the target polynucleotide between 300-fold to 1400-fold compared to whole genome sequencing. In some embodiments, the method enriches the base modification in the target polynucleotide between 300-fold to 1300-fold compared to whole genome sequencing. In some embodiments, the method enriches the base modification in the target polynucleotide between 300-fold to 1200-fold compared to whole genome sequencing. In some embodiments, the method enriches the base modification in the target polynucleotide between 300-fold to 1100-fold compared to whole genome sequencing. In some embodiments, the method enriches the base modification in the target polynucleotide between 300-fold to 1000-fold compared to whole genome sequencing. In some embodiments, the method enriches the base modification in the target polynucleotide between 300-fold to 900-fold compared to whole genome sequencing. In some embodiments, the method enriches the base modification in the target polynucleotide between 300-fold to 800-fold compared to whole genome sequencing. In some embodiments, the method enriches the base modification in the target polynucleotide between 300-fold to 700-fold compared to whole genome sequencing. In some embodiments, the method enriches the base modification in the target polynucleotide between 300-fold to 600-fold compared to whole genome sequencing. In some embodiments, the method enriches the base modification in the target polynucleotide between 300-fold to 500-fold compared to whole genome sequencing. In some embodiments, the method enriches the base modification in the target polynucleotide between 300-fold to 400-fold compared to whole genome sequencing.

### Compositions

Provided herein are compositions for use in a method of sequencing a nucleic acid (e.g., a target polynucleotide). In some embodiments, the compositions provided herein may be used in a method described herein including embodiments thereof. In some embodiments, the methods provided herein may produce a composition described herein including embodiments thereof. In some embodiments, the methods provided herein may produce a composition described herein including embodiments thereof at one or more steps in the method.

In an aspect is provided a hemisynthetic polynucleotide including a native nucleotide strand and a synthetic nucleotide strand, wherein the synthetic nucleotide strand is complementary to the native nucleotide strand, and wherein the hemisynthetic polynucleotide includes an adapter.

In some embodiments, the adapter is an oligonucleotide, a barcode, or a tag. In some embodiments, the adapter is an oligonucleotide. In some embodiments, the adapter is a barcode. In some embodiments, the adapter is a tag. In some embodiments, the adapter is RNA or includes RNA.

In some embodiments, the synthetic nucleotide strand includes a 3′ protective group or a 5′ protective group. In some embodiments, the synthetic nucleotide strand includes a 3′ protective group. In some embodiments, the synthetic nucleotide strand includes a 5′ protective group.

In some embodiments, the native strand includes a base modification.

In some embodiments, the base modification is an n1-methyl-pseudouridine, a pseudouridine (Ψ), a 5-methylcytosine (5mC), a 5-hydroxymethylcytosine (5hmC), a 4-methylcytosine (4mC), an N 6-methyladenine (6 mA), an N 6-methyladenosine (m6a), an N 1-methyladenosine (m1aA), a 7-methylguanine (m7G), a 2′-O-methylation (2′-O-Methyl), a 5-bromo-2′-deoxyuridine (BrdU), or a 5-ethynyl-2′-deoxyuridine (ErdU). In some embodiments, the base modification is an n1-methyl-pseudouridine. In some embodiments, the base modification is a pseudouridine (Ψ). In some embodiments, the base modification is a 5-methylcytosine (5mC). In some embodiments, the base modification is a 5-hydroxymethylcytosine (5hmC). In some embodiments, the base modification is a 4-methylcytosine (4mC). In some embodiments, the base modification is an N 6-methyladenine (6 mA). In some embodiments, the base modification is an N 6-methyladenosine (m6a). In some embodiments, the base modification is an N 1-methyladenosine (m1aA). In some embodiments, the base modification is a 7-methylguanine (m7G). In some embodiments, the base modification is a 2′-O-methylation (2′-O-Methyl). In some embodiments, the base modification is a 5-bromo-2′-deoxyuridine (BrdU). In some embodiments, the base modification is a 5-ethynyl-2′-deoxyuridine (ErdU).

In some embodiments, the 3′ protective group or the 5′ protective group prevents binding of a polymerase to the synthetic nucleotide strand. In some embodiments, the 3′ protective group prevents binding of a polymerase to the synthetic nucleotide strand. In some embodiments, the 5′ protective group prevents binding of a polymerase to the synthetic nucleotide strand.

In some embodiments, the synthetic nucleotide strand does not include an n1-methyl-pseudouridine, a pseudouridine (Ψ), a 5-methylcytosine (5mC), a 5-hydroxymethylcytosine (5hmC), a 4-methylcytosine (4mC), an N 6-methyladenine (6 mA), an N 6-methyladenosine (m6a), an N 1-methyladenosine (m1aA), a 7-methylguanine (m7G), a 2′-O-methylation (2′-O-Methyl), a 5-bromo-2′-deoxyuridine (BrdU), or a 5-ethynyl-2′-deoxyuridine (ErdU). In some embodiments, the synthetic nucleotide strand does not include an n1-methyl-pseudouridine. In some embodiments, the synthetic nucleotide strand does not include a pseudouridine (Ψ). In some embodiments, the synthetic nucleotide strand does not include a 5-methylcytosine (5mC). In some embodiments, the synthetic nucleotide strand does not include a 5-hydroxymethylcytosine (5hmC). In some embodiments, the synthetic nucleotide strand does not include a 4-methylcytosine (4mC). In some embodiments, the synthetic nucleotide strand does not include an N 6-methyladenine (6 mA). In some embodiments, the synthetic nucleotide strand does not include an N 6-methyladenosine (m6a). In some embodiments, the synthetic nucleotide strand does not include an N 1-methyladenosine (m1aA). In some embodiments, the synthetic nucleotide strand does not include a 7-methylguanine (m7G). In some embodiments, the synthetic nucleotide strand does not include a 2′-O-methylation (2′-O-Methyl). In some embodiments, the synthetic nucleotide strand does not include a 5-bromo-2′-deoxyuridine (BrdU). In some embodiments, the synthetic nucleotide strand does not include a 5-ethynyl-2′-deoxyuridine (ErdU).

### EXAMPLES

One skilled in the art would understand that the examples described herein are for the sole purpose of illustration, and that the present disclosure is not limited by this illustration.

### Example 1

Neurodegenerative diseases are marked by increased cell death of specific neuronal populations. For example, Alzheimer's disease results from a loss of cortical neurons, Parkinson's disease from the loss of dopaminergic neurons, and ALS from the loss of spinal motor neurons. However, although each disease has been classically linked to each cell type, the extent of secondary neuron loss among other populations is unknown. Here we sought to classify circulating cell-free DNA by neuronal cell-type of origin to measure the relative loss of each cell type in different types of neurodegeneration.

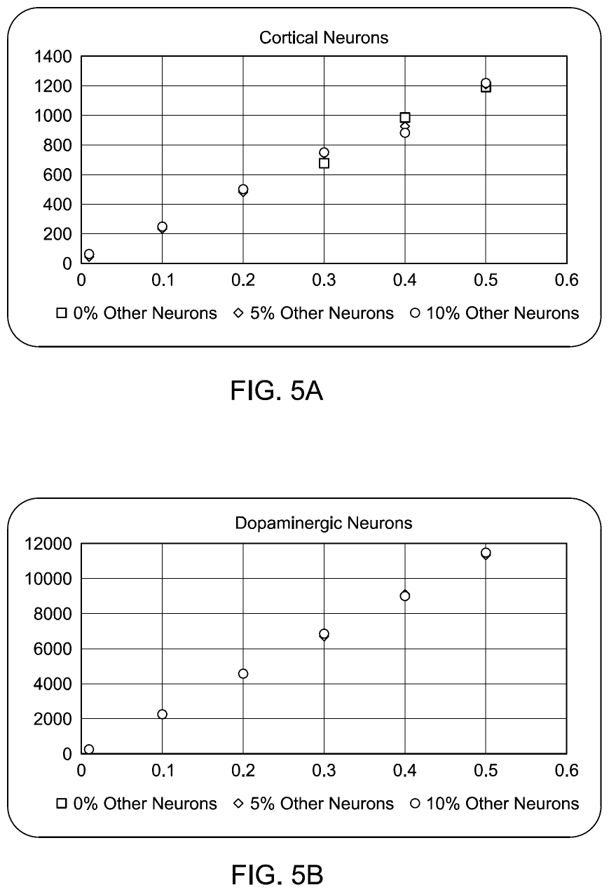

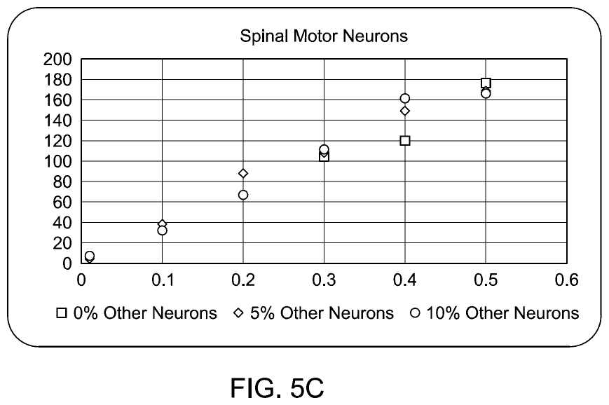

In order to classify reads, we first sequenced purified cortical neurons, purified dopaminergic neurons, and IPSC-derived spinal motor neurons using Nanopore sequencing. The resulting data was used to perform differential methylation analysis to identify genomic loci containing methylation signatures unique to a single cell-type. The number of informative loci include 4,152,224 informative CpGs from cortical neurons, 130,137 informative CpGs from dopaminergic neurons, and 1,427,252 informative CpGs from spinal motor neurons (each of these had >96% assignment accuracy). These sites were used to create a database containing informative genomic locations, as well as the cell type(s) associated with either methylation or non-methylation at that site. The model's accuracy was confirmed by sampling reads from each cell type and blood and differing ratios in the presence of 0, 10, and 15 percent other neuron types. Each synthetic sample showed a high correlation between classified read number and known percentage in the sample, and this was not affected by the presence of background from other neuron types (FIGS. 5A-5C).

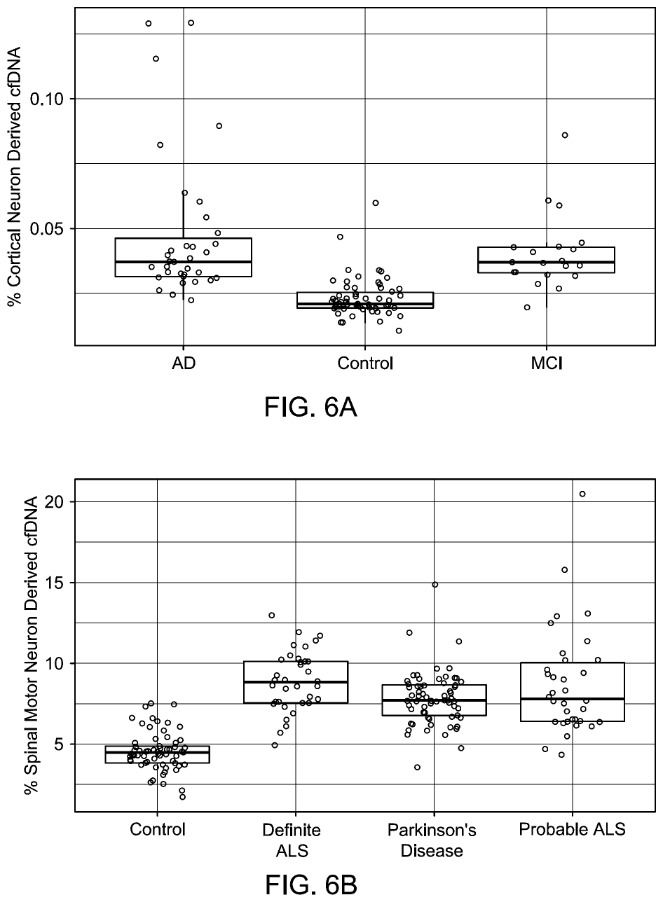

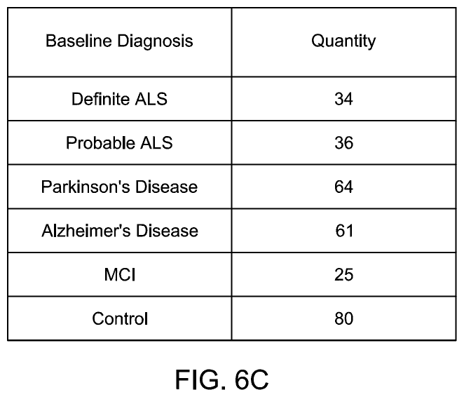

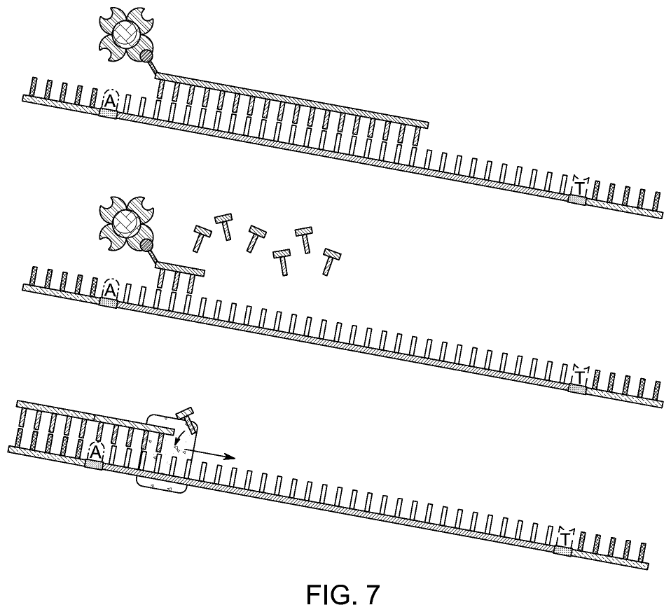

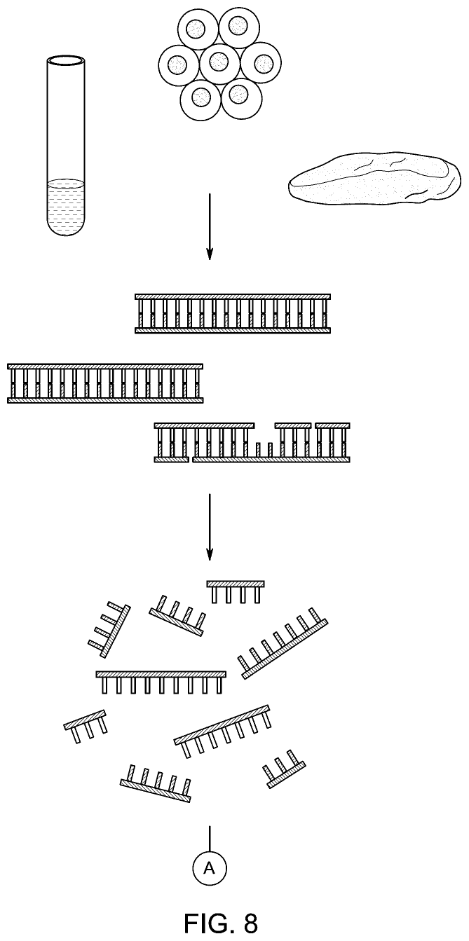

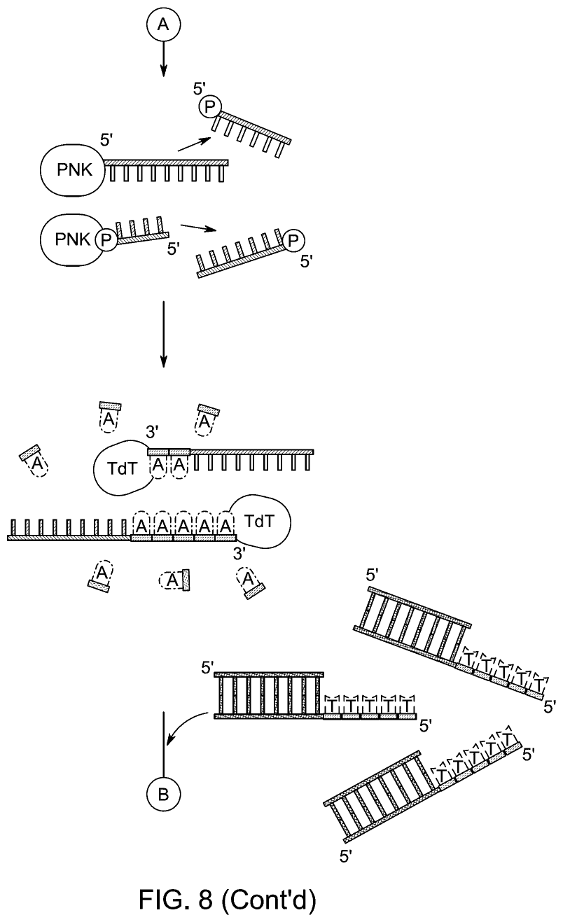

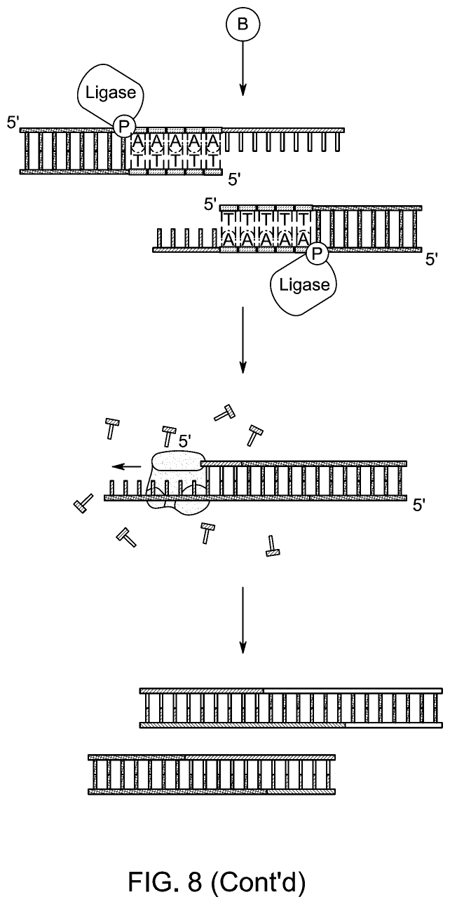

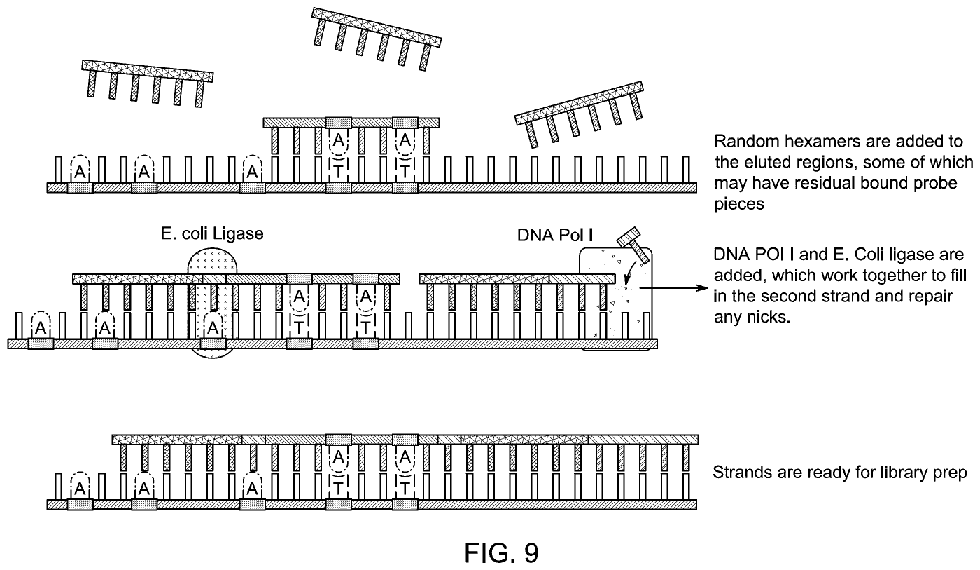

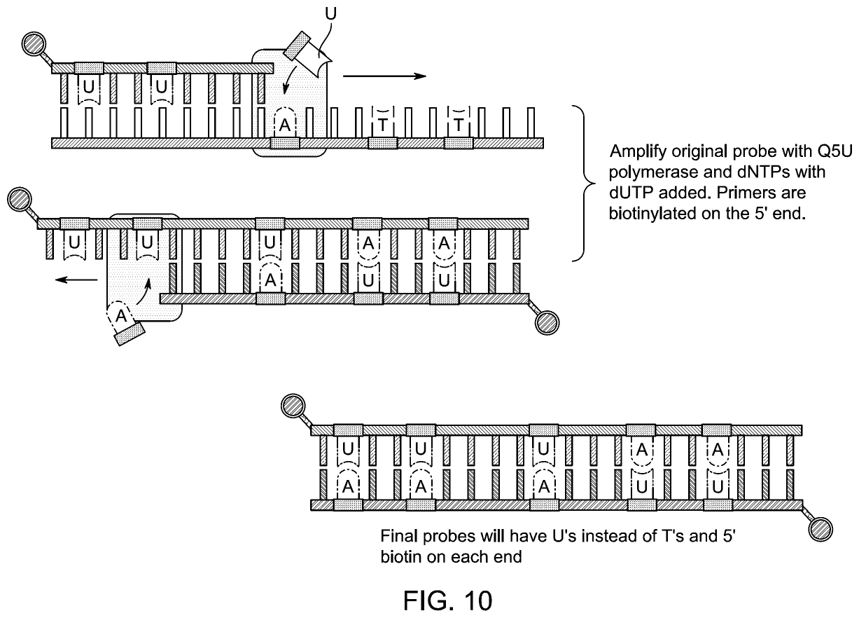

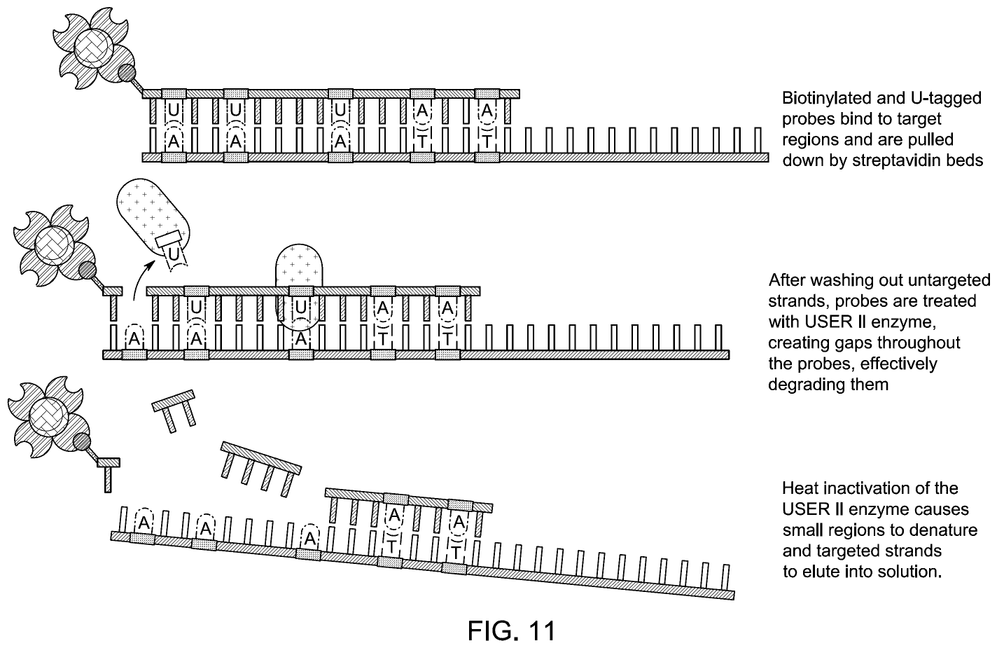

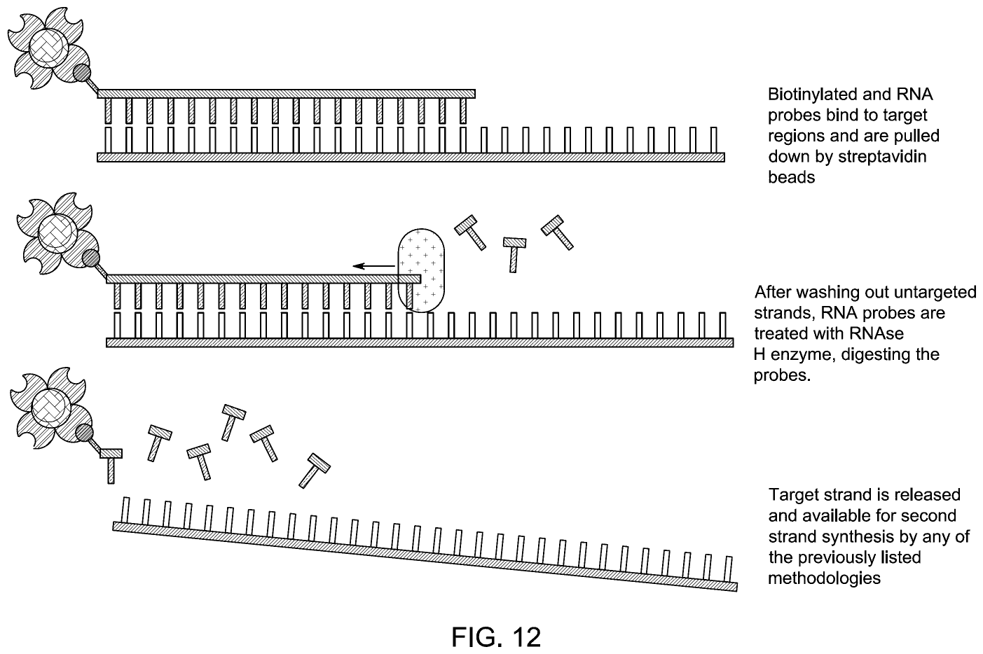

After model creation, cfDNA from patient cohorts with ALS, Parkinson's, and Alzheimer's, as well as both young healthy and age-matched controls (FIG. 6C), were sequenced to an average of 0.5× coverage using the procedure proposed here. Reads were then quality-filtered and mapped to the database of informative genomic coordinates. Passed reads were labeled as unclassified or a specific neuron type. Unclassified reads were discarded, and the percent of each neuron type was taken as a fraction of reads labeled as the cell type of interest divided by the total number of classifiable reads (FIGS. 6A-6B). In FIG. 6A, we specifically compared the fraction of cortical neuron-derived cfDNA in patients with Alzheimer's disease (AD) and Mild Cognitive Impairment (MCI) against healthy controls. We found that both neurodegenerative groups had higher percentages than the Controls. In another experiment, we looked at the fraction of cfDNA derived from spinal motor neurons in patients with ALS and Parkinson's and found that both disease groups contained biomarkers for increased spinal motor neuron death (FIG. 6B). Thus, our method allowed us to efficiently and accurately create methylation calls from cfDNA.

### Example 2: Methylation of Different Corn Genotypes

Epigenomic features, such as DNA methylation patterns, can serve as molecular signatures that distinguish organisms and reflect underlying genomic variation, developmental states, or environmental responses. The methods and compositions provided herein may be used to detect and analyze epigenomic features or patterns to differentiate genotypes.

In order to differentiate between corn (Zea mays) varieties, we sequenced genomic DNA using the method described herein. We first isolated and purified genomic DNA from different corn varieties and sequenced the purified DNA using Nanopore sequencing. The resulting data was used to perform a differential methylation analysis to identify genomic loci containing methylation signatures unique to a single corn variety. The methylated and unmethylated loci were used to create a database containing informative genomic locations, as well as the corn varieties associated with either methylation or non-methylation at that site. The model's accuracy was confirmed by sampling reads.

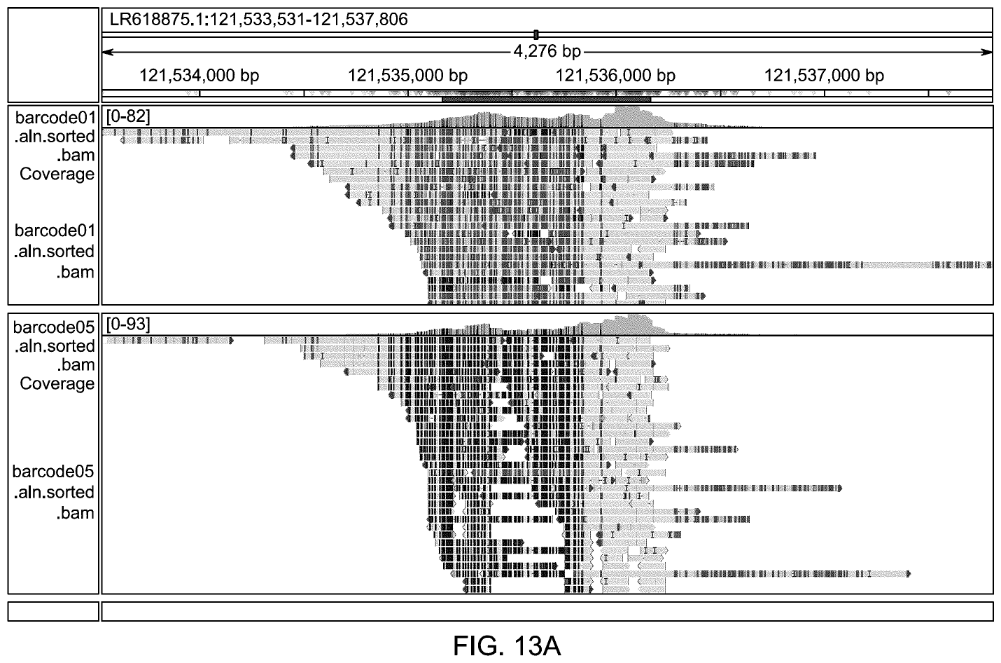

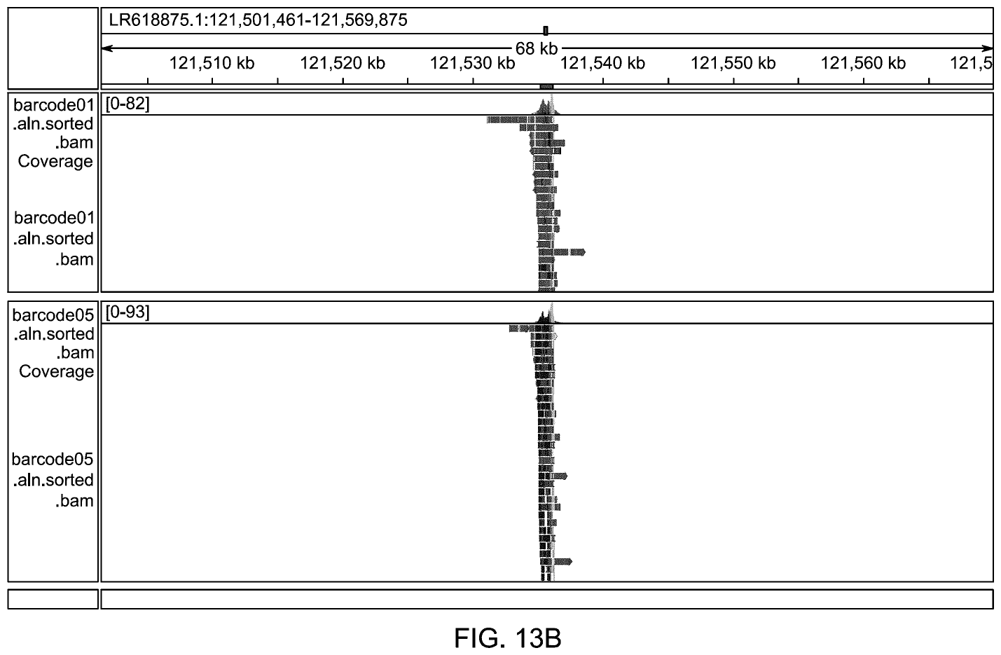

We were able to distinguish between different corn varieties by analyzing the methylation features of the genomic DNA produced by the sequencing method provided herein. Specifically, we were able to distinguish between different corn varieties with methylation (FIG. 13A, upper panel, gray) or unmethylation (FIG. 13A, lower panel, black) at specific targeted loci. Furthermore, we demonstrated that there was little to no off-target sequencing in regions near the targeted loci, thereby demonstrating our method's efficiency of enrichment of the targeted loci (FIG. 13B). These results underscore the method's capacity to efficiently resolve biologically relevant epigenomic variation across different corn varieties. These findings further illustrate the method's applicability in sequencing non-animal organisms and genomes.

### Example 3: Methylation of Polycystic Kidney Disease (PKD) Genes

Polycystic kidney disease (PKD) is associated with mutations and epigenomic alterations in the PKD1 and PKD2 genes, which regulate key pathways in renal development and function. Epigenomic features such as DNA methylation can influence gene expression and may serve as biomarkers for disease onset, progression, or therapeutic response. However, comprehensive analysis of these genes presents significant technical challenges due to their genomic architecture. PKD1, in particular, spans over 50 kb and contains regions of high GC content, extensive internal repeats, and six closely related pseudogenes with high sequence homology, complicating both amplification and alignment. PKD2 also exhibits elevated GC content and shares homology with related gene families.

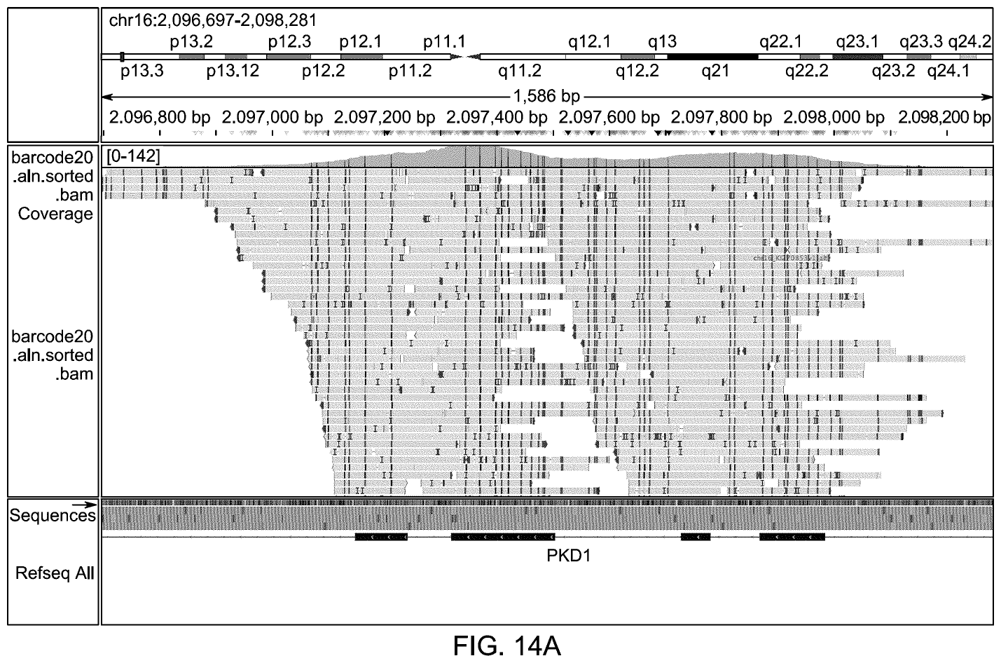

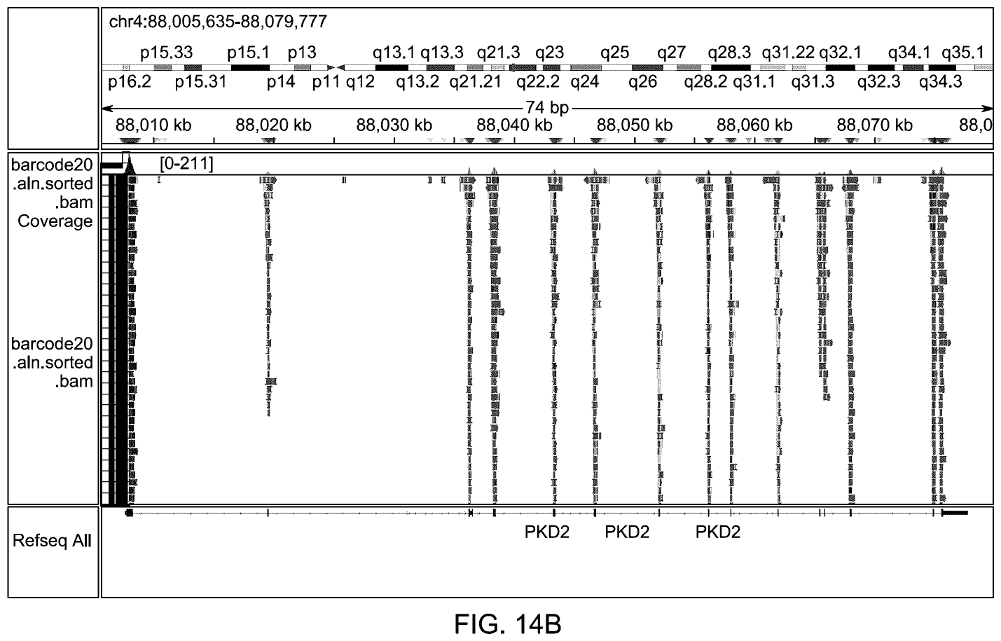

To demonstrate the robustness of the disclosed sequencing methods, genomic DNA was extracted from biological samples. The samples were processed using the sequencing methodology described herein, which enabled accurate detection of methylation marks even in GC-rich and repetitive regions. The method's long-read capability and optimized chemistry allowed for reliable differentiation between PKD1 and its pseudogenes, and for high-resolution mapping of methylation profiles across PKD1 (FIG. 14A). We also demonstrated that the method successfully enriched all exons of the PKD2 gene with little to no background.

These results demonstrate the effectiveness of the disclosed sequencing methodology in resolving epigenomic (e.g., methylation) patterns within complex genomic regions, including those with high GC content, repetitive elements, and pseudogene interference. The method's precision and adaptability make it well-suited for epigenomic profiling in clinically relevant genes, supporting applications in disease biomarker discovery, diagnostic assay development, and translational research across a range of genetic disorders.

### Example 4: Detection of Imprinted Genes

The methods provided herein may be applied to the detection of methylation patterns in imprinted genomic regions associated with a range of developmental and neurogenetic disorders. Imprinted genes exhibit parent-of-origin-specific methylation, and aberrant methylation in these regions has been implicated in conditions such as Angelman syndrome, Prader-Willi syndrome, Beckwith-Wiedemann syndrome, Silver-Russell syndrome, and the 15q11.2 BP1-BP2 deletion. These regions are known to contain tightly regulated methylation marks that are critical for normal gene expression and developmental timing.

Biological samples are collected from individuals suspected of having imprinting disorders. The methods described herein are used to target and enrich genomic regions known to harbor imprinted loci, enabling high-resolution detection of methylation states across these sites. The method's sensitivity to GC-rich and repetitive sequences, combined with its long-read capability, allows for accurate differentiation of methylated and unmethylated alleles, even in complex genomic contexts. This approach facilitates early and precise diagnosis of imprinting-related syndromes and supports the development of targeted gene therapies by providing detailed epigenomic maps of disease-relevant loci. The versatility of the methods provided herein provides a powerful tool for both diagnostic precision and therapeutic innovation in imprinting-related disorders or conditions.

### Example 5: Detection and Quantification of Sperm

The methods described herein may be used to detect and quantify sperm-derived DNA within complex biological mixtures such as seminal fluid. Sperm cells exhibit distinct methylation patterns at specific genomic loci, which differ from those found in somatic or epithelial cells present in the same fluid. These epigenomic signatures can be used to distinguish sperm DNA from non-sperm (e.g., background) DNA, enabling accurate assessment of sperm content and quality.

DNA is extracted from seminal fluid samples and the DNA is sequenced using the method disclosed herein. The disclosed method targets known methylation-enriched and methylation-depleted regions characteristic of sperm cells. By quantifying the number of reads mapping to these sperm-specific loci relative to background-associated loci, the method allows for reliable estimation of sperm DNA abundance. This approach leverages the method's sensitivity to methylation and its ability to resolve complex mixtures, supporting applications in fertility diagnostics, reproductive health monitoring, and research into epigenetic inheritance.

### Example 6: Epigenetic Stability in Sperm Cells

The methods described herein may be used to evaluate the epigenetic stability in sperm cells, which has been correlated with male fertility outcomes. Specific genomic loci in sperm exhibit tightly regulated methylation patterns that are essential for proper embryonic development and successful fertilization. Aberrant methylation or increased variability at these loci may serve as indicators of reduced fertility potential.

Sperm DNA is isolated from semen samples and the isolated DNA is sequenced using the methods provided herein. The method is used to selectively target genomic regions known to be epigenetically stable in fertile individuals. High coverage of these loci enables accurate quantification of methylation variability. By focusing sequencing efforts on these predefined regions, the method allows for cost-effective and high-resolution assessment of sperm epigenetic integrity. This approach utilizes the disclosed methods to support fertility diagnostics, informs assisted reproductive strategies, and contributes to research on epigenetic inheritance and reproductive health.

### Example 7: Detection of Neurological Disease

The methods described herein may be used to detect neurological disease by analyzing cell-free DNA (cfDNA) for methylation patterns specific to neuronal cell types. Each neuronal cell type exhibits a unique epigenomic signature, including distinct DNA methylation profiles that reflect its identity and functional state. During neurodegeneration or brain injury, dying neurons release DNA into the cerebrospinal fluid. That cfDNA can be detected in the bloodstream, and the presence and abundance of neuron-specific methylation patterns in cfDNA may serve as a biomarker for neuronal cell death.

cfDNA is extracted from blood samples of individuals suspected of having neurodegenerative or neuroinflammatory conditions. The disclosed sequencing methods are used to selectively target genomic regions known to be uniquely methylated or unmethylated in specific neuronal subtypes. By quantifying the methylation patterns in the cfDNA, the method enables sensitive and non-invasive detection of neuronal damage. The approach leverages the method's high resolution and ability to distinguish between methylation states in complex mixtures, supporting early diagnosis, disease monitoring, and therapeutic development in neurological disorders such as Alzheimer's disease, Parkinson's disease, and multiple sclerosis, among other neurological diseases.

### Example 8: Immune Cell Deconvolution

The methods described herein may be used to identify and quantify immune cell subpopulations based on cell-type-specific DNA methylation patterns. Each immune cell type and subtype—including T cells, B cells, monocytes, Natural Killer cells, and dendritic cells—is known to exhibit distinct epigenomic signatures that reflect lineage, activation state, and functional specialization. Changes in the relative abundance or methylation profiles of these cells may serve as indicators of immune status, disease progression, or therapeutic response.

DNA is extracted from whole blood or peripheral blood mononuclear cells (PBMCs) and the DNA is processed using the disclosed sequencing methods. The method targets genomic regions known to be differentially methylated across immune cell types. By quantifying the number of reads corresponding to these cell-specific loci, the method enables estimation of the relative proportions of each immune cell subtype within a sample. This approach supports applications in immunological research, disease monitoring (e.g., autoimmune disorders, infections, cancer), and personalized medicine by providing a non-invasive, epigenetically informed snapshot of immune cell composition.

### Example 9: Viral Detection in Crop Plants

The methods described herein may be used to detect a viral infection and its epigenetic consequences in crop plants (e.g., tubers). Viral exposure in certain plant species has been shown to induce stable, heritable changes in DNA methylation at specific genomic loci, which can negatively affect traits such as yield, disease resistance, and stress tolerance. Identifying these epigenetic alterations provides a means to assess the long-term impact of viral infection and to screen seed stock for quality assurance.

DNA is extracted from plant tissue samples and the extracted DNA is processed for sequencing using the methods disclosed herein. The method targets genomic regions known to undergo methylation changes in response to viral infection. By quantifying methylation states at these loci, the method enables detection of virus-induced epigenetic signatures and estimation of viral load. This targeted approach allows for high-resolution analysis with reduced sequencing cost, supporting the screening of seed lots and the removal of affected individuals prior to planting. This approach enhances agricultural productivity and informs breeding strategies by linking epigenomic profiles to crop performance.

### Example 10: Oligo Hybridization Enrichment for Native Strand Sequencing

Provided herein is an example method for sequencing a target polynucleotide. Other similar kits, reagents, enzymes, and/or buffers may be contemplated for use in the method described herein.

### Example 11: Sequencing Native Trands with RNA-Based Isolation Probes

Provided herein is an example method for sequencing a target polynucleotide using RNA-based isolation probes. Other similar kits, reagents, enzymes, and/or buffers may be contemplated for use in the method described herein.

### Example 12: Isolation Probe Library Amplification

Provided herein is an example method for amplifying a library of isolation probes using biotinylated primers. Other similar kits, reagents, enzymes, and/or buffers may be contemplated for use in the method described herein.

### Equipment

P10, P20, P200, and P1000 Pipettes
Thermocycler
Microcentrifuge or Vacuum Manifold
Gel Electrophoresis Apparatus
Gel Doc Imager

### Pre-Prepared Stock Solutions

Pool Amp Primer Solution

### Consumables

| | | Cat. | |
|---|---|---|---|
| Product | Vendor | Number | Link |
| Q5U DNA Polymerase | NEB | M0597L | Q5U Polymerase |
| | | | Product [symbol] |
| dUTP | NEB | N0459S | dUTP Product Page |
| dNTP Solution Set | NEB | N0446S | dNTP Solution Set |
| | | | Product [symbol] |
| Molecular Biology Grade | Genesee | 18-196 | Water Product Page |
| Water | | | |
| Clean & Concentrator-5 Kit | Genesee | 11-303 | C&C-5 Product Page |
| PCR Strip Tubes | Genesee | 27-125 | PCR Tubes Product |
| | | | Page |
| 1.5 mL Centrifuge Tubes | Genesee | 22-281S | 1.5 mL Tubes Product |
| | | | Page |
| Agarose | Genesee | 20-102QD | Agarose Product Page |
| TAE Buffer | Genesee | 20-194 | TAE Product Page |
| 50 bp Quick Load DNA | NEB | N0556S | 50 bp Ladder Product |
| Ladder (comes with | | | Page |
| Purple Loading Dye) | | | |
| Qubit DNA Broad Range | Thermo | Q32853 | Qubit DNA Kit |
| Kit | | | |

[symbol]

indicates data missing or illegible when filed

### Procedure

Plan 2 hours in a single day to complete.

12 Oligo Library Pools can be made simultaneously.

### P Embodiments

P Embodiment 1. A method for sequencing a target polynucleotide, the method comprising: (a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site; (b) contacting the adapter-target polynucleotide with a first polymerase under conditions promoting creation of a complementary adapter-target polynucleotide, wherein the complementary adapter-target polynucleotide comprises a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing comprises sequencing of the target polynucleotide sequence without sequencing the complementary adapter-target polynucleotide, thereby sequencing the target polynucleotide, optionally wherein the target polynucleotide comprises one or more base modifications.

P Embodiment 2. A method for detecting a base modification in a target polynucleotide, the method comprising: (a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site; (b) contacting the adapter-target polynucleotide with a first polymerase under conditions promoting creation of a complementary adapter-target polynucleotide, wherein the complementary adapter-target polynucleotide comprises a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing comprises sequencing of the target polynucleotide sequence without sequencing the complementary adapter-target polynucleotide, thereby detecting the base modification in the target polynucleotide.

P Embodiment 3. A method for quantifying a base modification in a target polynucleotide, the method comprising: (a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site; (b) contacting the adapter-target polynucleotide with a first polymerase under conditions promoting creation of a complementary adapter-target polynucleotide, wherein the complementary adapter-target polynucleotide comprises a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing comprises sequencing of the target polynucleotide sequence without sequencing the complementary adapter-target polynucleotide, thereby quantifying the base modification in the target polynucleotide.

P Embodiment 4. A method for sequencing a plurality of target polynucleotides, the method comprising: (a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters comprise a primer binding site; (b) contacting the plurality of adapter-target polynucleotides with a first polymerase under conditions promoting creation of a plurality of complementary adapter-target polynucleotides, wherein each of the complementary adapter-target polynucleotides comprises a 3′ protective group or a 5′ protective group; (c) contacting the plurality of adapter-target polynucleotides with a sequencing adapter in the presence of a second ligase under conditions promoting ligation of the sequencing adapter to the adapter-target polynucleotide to create a plurality of sequencing complexes; and (d) sequencing the plurality of sequencing complexes, wherein the sequencing comprises sequencing of the plurality of the target polynucleotide sequences without sequencing the plurality of complementary adapter-target polynucleotides, thereby sequencing the plurality of target polynucleotides, optionally wherein the plurality of target polynucleotides comprises one or more base modifications.

P Embodiment 5. A method for detecting a plurality of base modifications in a plurality of target polynucleotides, the method comprising: (a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters comprise a primer binding site; (b) contacting the plurality of adapter-target polynucleotides with a first polymerase under conditions promoting creation of a plurality of complementary adapter-target polynucleotides, wherein each of the complementary adapter-target polynucleotides comprises a 3′ protective group or a 5′ protective group; (c) contacting the plurality of adapter-target polynucleotides with a sequencing adapter in the presence of a second ligase under conditions promoting ligation of the sequencing adapter to the adapter-target polynucleotide to create a plurality of sequencing complexes; and (d) sequencing the plurality of sequencing complexes, wherein the sequencing comprises sequencing of the plurality of the target polynucleotide sequences without sequencing the plurality of complementary adapter-target polynucleotides, thereby detecting the plurality of base modifications in the plurality of target polynucleotides.

P Embodiment 6. A method for detecting a plurality of base modifications in a target polynucleotide, the method comprising: (a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site; (b) contacting the adapter-target polynucleotide with a first polymerase under conditions promoting creation of a complementary adapter-target polynucleotide, wherein the complementary adapter-target polynucleotide comprises a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing comprises sequencing of the target polynucleotide sequence without sequencing the complementary adapter-target polynucleotide, thereby detecting the plurality of base modifications in the target polynucleotide.

P Embodiment 7. A method for detecting a base modification in a plurality of target polynucleotides, the method comprising: (a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters comprise a primer binding site; (b) contacting the plurality of adapter-target polynucleotides with a first polymerase under conditions promoting creation of a plurality of complementary adapter-target polynucleotides, wherein each of the complementary adapter-target polynucleotides comprises a 3′ protective group or a 5′ protective group; (c) contacting the plurality of adapter-target polynucleotides with a sequencing adapter in the presence of a second ligase under conditions promoting ligation of the sequencing adapter to the adapter-target polynucleotide to create a plurality of sequencing complexes; and (d) sequencing the plurality of sequencing complexes, wherein the sequencing comprises sequencing of the plurality of the target polynucleotide sequences without sequencing the plurality of complementary adapter-target polynucleotides, thereby detecting the base modification in the plurality of target polynucleotides.

P Embodiment 8. The method of any one of the above P embodiments, further comprising contacting the adapter-target polynucleotides after step (b) with a solid support comprising a second portion of a cognate binding pair, under conditions promoting hybridization of the first portion of a cognate binding pair to the second portion of a cognate binding pair, to create a pulldown complex; isolating the pulldown complex; and cleaving the adapter-target polynucleotide to remove the cognate binding pair and solid support before step (c).

P Embodiment 9. The method of any one of the above P embodiments, wherein the first polymerase is a Q5U polymerase.

P Embodiment 10. The method of any one of the above P embodiments, wherein the complementary adapter-target polynucleotide comprises a uridine and does not comprise a thymidine.

P Embodiment 11. The method of any one of the above P embodiments, wherein the complementary adapter-target polynucleotide is contacted with a uridine-targeting enzyme, wherein the uridine-targeting enzyme excises the uridine residues.

P Embodiment 12. The method of any one of the above P embodiments, wherein step (b) comprises a DNA polymerase I and an E. coli ligase.

P Embodiment 13. The method of any one of the above P embodiments, further comprising: (e) analyzing the sequenced sequencing complex to detect the base modification.

P Embodiment 14. The method of any one of the above P embodiments, wherein the sequencing comprises identifying a base modification in the target polynucleotide.

P Embodiment 15. The method of any one of the above P embodiments, further comprising repeating steps (a) through (c) at least once with one or more additional adapters.

P Embodiment 16. The method of any one of the above P embodiments, wherein the target polynucleotide is isolated from a biological sample prior to step (a).

P Embodiment 17. The method of P embodiment 16, wherein the biological sample is from a human.

P Embodiment 18. The method of any one of the above P embodiments, wherein the target polynucleotide comprises genomic DNA (gDNA) or cell-free DNA (cfDNA).

P Embodiment 19. The method of any one of the above P embodiments, wherein the target polynucleotide is fragmented prior to step (a).

P Embodiment 20. The method of any one of the above P embodiments, wherein the one or more adapters further comprise a first barcode.

P Embodiment 21. The method of P embodiment 15, wherein the one or more additional adapters further comprise one or more additional barcodes.

P Embodiment 22. The method of any one of the above P embodiments, wherein the one or more adapters comprise streptavidin, biotin, maltose, maltose binding protein, glutathione, glutathione S-transferase, chitin, chitin binding protein, an aptamer, an antigen, SpyCatcher, SpyTag, or an antibody.

P Embodiment 23. The method of any one of the above P embodiments, wherein the one or more adapters target the same region of the target polynucleotide.

P Embodiment 24. The method of any one of the above P embodiments, wherein the one or more adapters target different regions of the target polynucleotide.

P Embodiment 25. The method of any one of the above P embodiments, wherein the solid support comprises streptavidin, biotin, maltose, maltose binding protein, glutathione, glutathione S-transferase, chitin, chitin binding protein, an aptamer, an antigen, SpyCatcher, SpyTag, or an antibody.

P Embodiment 26. The method of any one of the above P embodiments, wherein the solid support is a bead or a column.

P Embodiment 27. The method of P embodiment 26, wherein the bead is paramagnetic.

P Embodiment 28. The method of any one of the above P embodiments, wherein the adapter-target polynucleotide is eluted from the pulldown complex with NaOH, heat, a strand-displacing polymerase, a polymerase with exonuclease activity, or a uridine-targeting enzyme.

P Embodiment 29. The method of any one of the above P embodiments, wherein the 3′ protective group or the 5′ protective group prevents sequencing of the complementary adapter-target polynucleotide.

P Embodiment 30. The method of any one of the above P embodiments, wherein the 5′ protective group comprises an aldehyde, an amine, or a thiol.

P Embodiment 31. The method of any one of the above P embodiments, wherein the 3′ protective group comprises a nucleotide overhang.

P Embodiment 32. The method of any one of the above P embodiments, the primer does not comprise a 5′ phosphate group.

P Embodiment 33. The method of any one of the above P embodiments, the sequencing adapter comprises a second barcode.

P Embodiment 34. The method of any one of the above P embodiments, wherein the base modification comprises a 5-methylcytosine (5mC), a 5-hydroxymethylcytosine (5hmC), a N 6-methyladenine (6 mA), a 7-methylguanine (m7G), 2′-O-methylation (2′-O-Methyl), or a bromodeoxyuridine (BrdU).

P Embodiment 35. The method of any one of the above P embodiments, wherein the method does not comprise bisulfite conversion or amplification of the target polynucleotide.

P Embodiment 36. The method of any one of the above P embodiments, wherein the method does not comprise reduced representation bisulfite sequencing (RRBS) or reduced representation methylation sequencing (RRMS).

P Embodiment 37. The method of any one of the above P embodiments, wherein the first ligase or the second ligase is a T4 DNA ligase, a Quick ligase, a T3 ligase, a T7 ligase, or a Tag ligase.

P Embodiment 38. The method of any one of the above P embodiments, wherein the first polymerase is a Taq polymerase, a BST polymerase, a Sulfolobus polymerase, a Therminator polymerase, a Klenow polymerase, a deep vent polymerase.

P Embodiment 39. The method of any one of the above P embodiments, wherein sequencing is performed on a sequencer capable of identifying modified bases.

P Embodiment 40. The method of any one of the above P embodiments, wherein the base modification in the target polynucleotide is retained until sequencing.

P Embodiment 41. The method of any one of the above P embodiments, wherein the method enriches the base modification in the target polynucleotide between about 300-fold to about 2400-fold compared to whole genome sequencing.

P Embodiment 42. The method of any one of the above P embodiments, wherein the adapter-target polynucleotide is eluted from the pulldown complex with a buffer comprising a ribonuclease (RNase).

### Embodiments

Embodiment 1. A method for sequencing a target polynucleotide, the method comprising: (a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site; (b) contacting the adapter-target polynucleotide with a first polymerase under conditions promoting creation of a complementary adapter-target polynucleotide, wherein the complementary adapter-target polynucleotide comprises a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing comprises sequencing of the target polynucleotide sequence without sequencing the complementary adapter-target polynucleotide, thereby sequencing the target polynucleotide, optionally wherein the target polynucleotide comprises one or more base modifications.

Embodiment 2. A method for detecting a base modification in a target polynucleotide, the method comprising: (a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site; (b) contacting the adapter-target polynucleotide with a first polymerase under conditions promoting creation of a complementary adapter-target polynucleotide, wherein the complementary adapter-target polynucleotide comprises a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing comprises sequencing of the target polynucleotide sequence without sequencing the complementary adapter-target polynucleotide, thereby detecting the base modification in the target polynucleotide.

Embodiment 3. A method for quantifying a base modification in a target polynucleotide, the method comprising: (a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site; (b) contacting the adapter-target polynucleotide with a first polymerase under conditions promoting creation of a complementary adapter-target polynucleotide, wherein the complementary adapter-target polynucleotide comprises a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing comprises sequencing of the target polynucleotide sequence without sequencing the complementary adapter-target polynucleotide, thereby quantifying the base modification in the target polynucleotide.

Embodiment 4. A method for sequencing a plurality of target polynucleotides, the method comprising: (a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters comprise a primer binding site; (b) contacting the plurality of adapter-target polynucleotides with a first polymerase under conditions promoting creation of a plurality of complementary adapter-target polynucleotides, wherein each of the complementary adapter-target polynucleotides comprises a 3′ protective group or a 5′ protective group; (c) contacting the plurality of adapter-target polynucleotides with a sequencing adapter in the presence of a second ligase under conditions promoting ligation of the sequencing adapter to the adapter-target polynucleotide to create a plurality of sequencing complexes; and (d) sequencing the plurality of sequencing complexes, wherein the sequencing comprises sequencing of the plurality of the target polynucleotide sequences without sequencing the plurality of complementary adapter-target polynucleotides, thereby sequencing the plurality of target polynucleotides, optionally wherein the plurality of target polynucleotides comprises one or more base modifications.

Embodiment 5. A method for detecting a plurality of base modifications in a plurality of target polynucleotides, the method comprising: (a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters comprise a primer binding site; (b) contacting the plurality of adapter-target polynucleotides with a first polymerase under conditions promoting creation of a plurality of complementary adapter-target polynucleotides, wherein each of the complementary adapter-target polynucleotides comprises a 3′ protective group or a 5′ protective group; (c) contacting the plurality of adapter-target polynucleotides with a sequencing adapter in the presence of a second ligase under conditions promoting ligation of the sequencing adapter to the adapter-target polynucleotide to create a plurality of sequencing complexes; and (d) sequencing the plurality of sequencing complexes, wherein the sequencing comprises sequencing of the plurality of the target polynucleotide sequences without sequencing the plurality of complementary adapter-target polynucleotides, thereby detecting the plurality of base modifications in the plurality of target polynucleotides.

Embodiment 6. A method for detecting a plurality of base modifications in a target polynucleotide, the method comprising: (a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site; (b) contacting the adapter-target polynucleotide with a first polymerase under conditions promoting creation of a complementary adapter-target polynucleotide, wherein the complementary adapter-target polynucleotide comprises a 3′ protective group or a 5′ protective group; (c) contacting the adapter-target polynucleotide with a sequencing adapter in the presence of a second ligase under conditions promoting ligation of the sequencing adapter to the adapter-target polynucleotide to create a sequencing complex; and (d) sequencing the sequencing complex, wherein the sequencing comprises sequencing of the target polynucleotide sequence without sequencing the complementary adapter-target polynucleotide, thereby detecting the plurality of base modifications in the target polynucleotide.

Embodiment 7. A method for detecting a base modification in a plurality of target polynucleotides, the method comprising: (a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters comprise a primer binding site; (b) contacting the plurality of adapter-target polynucleotides with a first polymerase under conditions promoting creation of a plurality of complementary adapter-target polynucleotides, wherein each of the complementary adapter-target polynucleotides comprises a 3′ protective group or a 5′ protective group; (c) contacting the plurality of adapter-target polynucleotides with a sequencing adapter in the presence of a second ligase under conditions promoting ligation of the sequencing adapter to the adapter-target polynucleotide to create a plurality of sequencing complexes; and (d) sequencing the plurality of sequencing complexes, wherein the sequencing comprises sequencing of the plurality of the target polynucleotide sequences without sequencing the plurality of complementary adapter-target polynucleotides, thereby detecting the base modification in the plurality of target polynucleotides.

Embodiment 8. The method of any one of the above embodiments, further comprising contacting the target polynucleotide with one or more isolation probes prior to step (a) under conditions promoting hybridization of the one or more isolation probes to the target polynucleotide, to create an isolation probe-target polynucleotide, wherein the one or more isolation probes are complementary to at least a portion of the target polynucleotide.

Embodiment 9. The method of embodiment 8, wherein the one or more isolation probes comprise a uridine nucleotide, and wherein the one or more isolation probes do not comprise a thymidine nucleotide.

Embodiment 10. The method of embodiment 8 or 9, wherein the one or more isolation probes further comprises a first portion of a cognate binding pair.

Embodiment 11. The method of embodiment 10, further comprising contacting the isolation probe-target polynucleotide with a solid support comprising a second portion of the cognate binding pair, under conditions promoting hybridization of the first portion of the cognate binding pair to the second portion of the cognate binding pair, to create a pulldown complex; isolating the pulldown complex; and contacting the pulldown complex with a non-standard DNA nucleotide-targeting enzyme, thereby cleaving the one or more isolation probes to create a plurality of cleaved isolation probes.

Embodiment 12. The method of embodiment 11, wherein the non-standard DNA nucleotide-targeting enzyme is a uridine-targeting endonuclease, wherein the uridine-targeting endonuclease excises the uridine nucleotides in the one or more isolation probes.

Embodiment 13. The method of embodiment 12, wherein the uridine-targeting endonuclease is a USER II endonuclease.

Embodiment 14. The method of any one of the above embodiments, wherein the one or more adapters comprise a first portion of a cognate binding pair.

Embodiment 15. The method of embodiment 14, further comprising contacting the adapter-target polynucleotides after step (b) with a solid support comprising a second portion of the cognate binding pair, under conditions promoting hybridization of the first portion of the cognate binding pair to the second portion of the cognate binding pair, to create a pulldown complex; isolating the pulldown complex; and cleaving the adapter-target polynucleotide to remove the cognate binding pair and solid support before step (d).

Embodiment 16. The method of any one of the above embodiments, wherein the first polymerase is a Q5U polymerase.

Embodiment 17. The method of any one of the above embodiments, wherein the complementary adapter-target polynucleotide comprises a uridine and does not comprise a thymidine.

Embodiment 18. The method of any one of the above embodiments, wherein the complementary adapter-target polynucleotide is contacted with a uridine-targeting enzyme, wherein the uridine-targeting enzyme excises the uridine residues.

Embodiment 19. The method of any one of the above embodiments, wherein step (b) comprises a DNA polymerase I and an E. coli ligase.

Embodiment 20. The method of any one of the above embodiments, further comprising: (e) analyzing the sequenced sequencing complex to detect the base modification.

Embodiment 21. The method of any one of the above embodiments, wherein the sequencing comprises identifying a base modification in the target polynucleotide.

Embodiment 22. The method of any one of the above embodiments, further comprising repeating steps (a) through (e) at least once with one or more additional adapters.

Embodiment 23. The method of any one of the above embodiments, wherein the target polynucleotide is isolated from a biological sample prior to step (a).

Embodiment 24. The method of embodiment 23, wherein the biological sample is from a human.

Embodiment 25. The method of any one of the above embodiments, wherein the target polynucleotide comprises genomic DNA (gDNA) or cell-free DNA (cfDNA).

Embodiment 26. The method of any one of the above embodiments, wherein the target polynucleotide is fragmented prior to step (a).

Embodiment 27. The method of any one of the above embodiments, wherein the one or more adapters further comprise a first barcode.

Embodiment 28. The method of embodiment 22, wherein the one or more additional adapters further comprise one or more additional barcodes.

Embodiment 29. The method of any one of the above embodiments, wherein the one or more adapters comprise streptavidin, biotin, maltose, maltose binding protein, glutathione, glutathione S-transferase, chitin, chitin binding protein, an aptamer, an antigen, SpyCatcher, SpyTag, or an antibody.

Embodiment 30. The method of any one of the above embodiments, wherein the one or more adapters target the same region of the target polynucleotide.

Embodiment 31. The method of any one of the above embodiments, wherein the one or more adapters target different regions of the target polynucleotide.

Embodiment 32. The method of any one of the above embodiments, wherein the solid support comprises streptavidin, biotin, maltose, maltose binding protein, glutathione, glutathione S-transferase, chitin, chitin binding protein, an aptamer, an antigen, SpyCatcher, SpyTag, or an antibody.

Embodiment 33. The method of any one of the above embodiments, wherein the solid support is a bead or a column.

Embodiment 34. The method of embodiment 33, wherein the bead is paramagnetic.

Embodiment 35. The method of any one of the above embodiments, wherein the adapter-target polynucleotide is eluted from the pulldown complex with NaOH, heat, a strand-displacing polymerase, a polymerase with exonuclease activity, or a uridine-targeting enzyme.

Embodiment 36. The method of any one of the above embodiments, wherein the 3′ protective group or the 5′ protective group prevents sequencing of the complementary adapter-target polynucleotide.

Embodiment 37. The method of any one of the above embodiments, wherein the 5′ protective group comprises an aldehyde, an amine, or a thiol.

Embodiment 38. The method of any one of the above embodiments, wherein the 3′ protective group comprises a nucleotide overhang.

Embodiment 39. The method of any one of the above embodiments, the primer does not comprise a 5′ phosphate group.

Embodiment 40. The method of any one of the above embodiments, the sequencing adapter comprises a second barcode.

Embodiment 41. The method of any one of the above embodiments, wherein the base modification comprises an n1-methyl-pseudouridine, a pseudouridine (Ψ), a 5-methylcytosine (5mC), a 5-hydroxymethylcytosine (5hmC), a 4-methylcytosine (4mC), an N 6-methyladenine (6 mA), an N 6-methyladenosine (m6a), an N 1-methyladenosine (m1aA), a 7-methylguanine (m7G), 2′-O-methylation (2′-O-Methyl), a 5-bromo-2′-deoxyuridine (BrdU), or a 5-ethynyl-2′-deoxyuridine (ErdU).

Embodiment 42. The method of any one of the above embodiments, wherein the method does not comprise bisulfite conversion or amplification of the target polynucleotide.

Embodiment 43. The method of any one of the above embodiments, wherein the method does not comprise reduced representation bisulfite sequencing (RRBS) or reduced representation methylation sequencing (RRMS).

Embodiment 44. The method of any one of the above embodiments, wherein the first ligase or the second ligase is a T4 DNA ligase, a Quick ligase, a T3 ligase, a T7 ligase, or a Tag ligase.

Embodiment 45. The method of any one of the above embodiments, wherein the first polymerase is a Taq polymerase, a BST polymerase, a Sulfolobus polymerase, a Therminator polymerase, a Klenow polymerase, a deep vent polymerase.

Embodiment 46. The method of any one of the above embodiments, wherein sequencing is performed on a sequencer capable of identifying modified bases.

Embodiment 47. The method of any one of the above embodiments, wherein the base modification in the target polynucleotide is retained until sequencing.

Embodiment 48. The method of any one of the above embodiments, wherein the method enriches the base modification in the target polynucleotide between about 300-fold to about 2400-fold compared to whole genome sequencing.

Embodiment 49. The method of any one of the above embodiments, wherein the adapter-target polynucleotide is eluted from the pulldown complex with a buffer comprising a ribonuclease (RNase).

What is claimed is:

1. A method for sequencing a target polynucleotide, the method comprising:
(a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site;
(b) contacting the adapter-target polynucleotide with a first polymerase under conditions promoting creation of a complementary adapter-target polynucleotide, wherein the complementary adapter-target polynucleotide comprises a 3′ protective group or a 5′ protective group;
(c) contacting the adapter-target polynucleotide with a sequencing adapter in the presence of a second ligase under conditions promoting ligation of the sequencing adapter to the adapter-target polynucleotide to create a sequencing complex; and
(d) sequencing the sequencing complex, wherein the sequencing comprises sequencing of the target polynucleotide sequence without sequencing the complementary adapter-target polynucleotide,
thereby sequencing the target polynucleotide, optionally wherein the target polynucleotide comprises one or more base modifications.

2. A method for detecting a base modification in a target polynucleotide, the method comprising:
(a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site;
(b) contacting the adapter-target polynucleotide with a first polymerase under conditions promoting creation of a complementary adapter-target polynucleotide, wherein the complementary adapter-target polynucleotide comprises a 3′ protective group or a 5′ protective group;
(c) contacting the adapter-target polynucleotide with a sequencing adapter in the presence of a second ligase under conditions promoting ligation of the sequencing adapter to the adapter-target polynucleotide to create a sequencing complex; and
(d) sequencing the sequencing complex, wherein the sequencing comprises sequencing of the target polynucleotide sequence without sequencing the complementary adapter-target polynucleotide,
thereby detecting the base modification in the target polynucleotide.

3. A method for quantifying a base modification in a target polynucleotide, the method comprising:
(a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site;
(b) contacting the adapter-target polynucleotide with a first polymerase under conditions promoting creation of a complementary adapter-target polynucleotide, wherein the complementary adapter-target polynucleotide comprises a 3′ protective group or a 5′ protective group;
(c) contacting the adapter-target polynucleotide with a sequencing adapter in the presence of a second ligase under conditions promoting ligation of the sequencing adapter to the adapter-target polynucleotide to create a sequencing complex; and
(d) sequencing the sequencing complex, wherein the sequencing comprises sequencing of the target polynucleotide sequence without sequencing the complementary adapter-target polynucleotide,
thereby quantifying the base modification in the target polynucleotide.

4. A method for sequencing a plurality of target polynucleotides, the method comprising:
(a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters comprise a primer binding site;
(b) contacting the plurality of adapter-target polynucleotides with a first polymerase under conditions promoting creation of a plurality of complementary adapter-target polynucleotides, wherein each of the complementary adapter-target polynucleotides comprises a 3′ protective group or a 5′ protective group;
(c) contacting the plurality of adapter-target polynucleotides with a sequencing adapter in the presence of a second ligase under conditions promoting ligation of the sequencing adapter to the adapter-target polynucleotide to create a plurality of sequencing complexes; and
(d) sequencing the plurality of sequencing complexes, wherein the sequencing comprises sequencing of the plurality of the target polynucleotide sequences without sequencing the plurality of complementary adapter-target polynucleotides,
thereby sequencing the plurality of target polynucleotides, optionally wherein the plurality of target polynucleotides comprises one or more base modifications.

5. A method for detecting a plurality of base modifications in a plurality of target polynucleotides, the method comprising:
(a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters comprise a primer binding site;
(b) contacting the plurality of adapter-target polynucleotides with a first polymerase under conditions promoting creation of a plurality of complementary adapter-target polynucleotides, wherein each of the complementary adapter-target polynucleotides comprises a 3′ protective group or a 5′ protective group;
(c) contacting the plurality of adapter-target polynucleotides with a sequencing adapter in the presence of a second ligase under conditions promoting ligation of the sequencing adapter to the adapter-target polynucleotide to create a plurality of sequencing complexes; and
(d) sequencing the plurality of sequencing complexes, wherein the sequencing comprises sequencing of the plurality of the target polynucleotide sequences without sequencing the plurality of complementary adapter-target polynucleotides,
thereby detecting the plurality of base modifications in the plurality of target polynucleotides.

6. A method for detecting a plurality of base modifications in a target polynucleotide, the method comprising:
(a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site;
(b) contacting the adapter-target polynucleotide with a first polymerase under conditions promoting creation of a complementary adapter-target polynucleotide, wherein the complementary adapter-target polynucleotide comprises a 3′ protective group or a 5′ protective group;
(c) contacting the adapter-target polynucleotide with a sequencing adapter in the presence of a second ligase under conditions promoting ligation of the sequencing adapter to the adapter-target polynucleotide to create a sequencing complex; and
(d) sequencing the sequencing complex, wherein the sequencing comprises sequencing of the target polynucleotide sequence without sequencing the complementary adapter-target polynucleotide,
thereby detecting the plurality of base modifications in the target polynucleotide.

7. A method for detecting a base modification in a plurality of target polynucleotides, the method comprising:
(a) contacting the plurality of target polynucleotides with one or more adapters in the presence of a first ligase under conditions promoting ligation of the one or more adapters to the plurality of target polynucleotide to create a plurality of adapter-target polynucleotides, wherein the one or more adapters comprise a primer binding site;
(b) contacting the plurality of adapter-target polynucleotides with a first polymerase under conditions promoting creation of a plurality of complementary adapter-target polynucleotides, wherein each of the complementary adapter-target polynucleotides comprises a 3′ protective group or a 5′ protective group;
(c) contacting the plurality of adapter-target polynucleotides with a sequencing adapter in the presence of a second ligase under conditions promoting ligation of the sequencing adapter to the adapter-target polynucleotide to create a plurality of sequencing complexes; and
(d) sequencing the plurality of sequencing complexes, wherein the sequencing comprises sequencing of the plurality of the target polynucleotide sequences without sequencing the plurality of complementary adapter-target polynucleotides,
thereby detecting the base modification in the plurality of target polynucleotides.

8. The method of claim 1, further comprising contacting the target polynucleotide with one or more isolation probes prior to step (a) under conditions promoting hybridization of the one or more isolation probes to the target polynucleotide, to create an isolation probe-target polynucleotide, wherein the one or more isolation probes are complementary to at least a portion of the target polynucleotide.

9. The method of claim 8, wherein the one or more isolation probes comprise a uridine nucleotide, and wherein the one or more isolation probes do not comprise a thymidine nucleotide.

10. The method of claim 8, wherein the one or more isolation probes further comprises a first portion of a cognate binding pair.

11. The method of claim 10, further comprising contacting the isolation probe-target polynucleotide with a solid support comprising a second portion of the cognate binding pair, under conditions promoting hybridization of the first portion of the cognate binding pair to the second portion of the cognate binding pair, to create a pulldown complex;
isolating the pulldown complex; and
contacting the pulldown complex with a non-standard DNA nucleotide-targeting enzyme, thereby cleaving the one or more isolation probes to create a plurality of cleaved isolation probes.

12. The method of claim 11, wherein the non-standard DNA nucleotide-targeting enzyme is a uridine-targeting endonuclease, wherein the uridine-targeting endonuclease excises the uridine nucleotides in the one or more isolation probes.

13. The method of claim 12, wherein the uridine-targeting endonuclease is a USER II endonuclease.

14. The method of claim 1, wherein the one or more adapters comprise a first portion of a cognate binding pair.

15. The method of claim 14, further comprising contacting the adapter-target polynucleotides after step (b) with a solid support comprising a second portion of the cognate binding pair, under conditions promoting hybridization of the first portion of the cognate binding pair to the second portion of the cognate binding pair, to create a pulldown complex;
isolating the pulldown complex; and
cleaving the adapter-target polynucleotide to remove the cognate binding pair and solid support before step (d).

16. The method of claim 1, wherein the first polymerase is a Q5U polymerase.

17. The method of claim 1, wherein the complementary adapter-target polynucleotide comprises a uridine and does not comprise a thymidine.

18. The method of claim 17, wherein the complementary adapter-target polynucleotide is contacted with a uridine-targeting enzyme, wherein the uridine-targeting enzyme excises the uridine residues.

19. The method of claim 1, wherein step (b) comprises a DNA polymerase I and an E. coli ligase.

20. The method of claim 1, further comprising:
(e) analyzing the sequenced sequencing complex to detect the base modification.

21. The method of claim 1, wherein the sequencing comprises identifying a base modification in the target polynucleotide.

22. The method of claim 20, further comprising repeating steps (a) through (e) at least once with one or more additional adapters.

23. The method of claim 1, wherein the target polynucleotide is isolated from a biological sample prior to step (a).

24. The method of claim 23, wherein the biological sample is from a human.

25. The method of claim 1, wherein the target polynucleotide comprises genomic DNA (gDNA) or cell-free DNA (cfDNA).

26. The method of claim 1, wherein the target polynucleotide is fragmented prior to step (a).

27. The method of claim 1, wherein the one or more adapters further comprise a first barcode.

28. The method of claim 22, wherein the one or more additional adapters further comprise one or more additional barcodes.

29. The method of claim 1, wherein the one or more adapters comprise streptavidin, biotin, maltose, maltose binding protein, glutathione, glutathione S-transferase, chitin, chitin binding protein, an aptamer, an antigen, SpyCatcher, SpyTag, or an antibody.

30. The method of claim 1, wherein the one or more adapters target the same region of the target polynucleotide.

31. The method of claim 1, wherein the one or more adapters target different regions of the target polynucleotide.

32. The method of claim 1, wherein the solid support comprises streptavidin, biotin, maltose, maltose binding protein, glutathione, glutathione S-transferase, chitin, chitin binding protein, an aptamer, an antigen, SpyCatcher, SpyTag, or an antibody.

33. The method of claim 32, wherein the solid support is a bead or a column.

34. The method of claim 33, wherein the bead is paramagnetic.

35. The method of claim 1, wherein the adapter-target polynucleotide is eluted from the pulldown complex with NaOH, heat, a strand-displacing polymerase, a polymerase with exonuclease activity, or a uridine-targeting enzyme.

36. The method of claim 1, wherein the 3′ protective group or the 5′ protective group prevents sequencing of the complementary adapter-target polynucleotide.

37. The method of claim 36, wherein the 5′ protective group comprises an aldehyde, an amine, or a thiol.

38. The method of claim 36, wherein the 3′ protective group comprises a nucleotide overhang.

39. The method of claim 1, the primer does not comprise a 5′ phosphate group.

40. The method of claim 1, the sequencing adapter comprises a second barcode.

41. The method of claim 1, wherein the base modification comprises an n1-methyl-pseudouridine, a pseudouridine (Ψ), a 5-methylcytosine (5mC), a 5-hydroxymethylcytosine (5hmC), a 4-methylcytosine (4mC), an N 6-methyladenine (6 mA), an N 6-methyladenosine (m6a), an N 1-methyladenosine (m1aA), a 7-methylguanine (m7G), 2′-O-methylation (2′-O-Methyl), a 5-bromo-2′-deoxyuridine (BrdU), or a 5-ethynyl-2′-deoxyuridine (ErdU).

42. The method of claim 1, wherein the method does not comprise bisulfite conversion or amplification of the target polynucleotide.

43. The method of claim 1, wherein the method does not comprise reduced representation bisulfite sequencing (RRBS) or reduced representation methylation sequencing (RRMS).

44. The method of claim 1, wherein the first ligase or the second ligase is a T4 DNA ligase, a Quick ligase, a T3 ligase, a T7 ligase, or a Tag ligase.

45. The method of claim 1, wherein the first polymerase is a Taq polymerase, a BST polymerase, a Sulfolobus polymerase, a Therminator polymerase, a Klenow polymerase, a deep vent polymerase.

46. The method of claim 1, wherein sequencing is performed on a sequencer capable of identifying modified bases.

47. The method of claim 1, wherein the base modification in the target polynucleotide is retained until sequencing.

48. The method of claim 1, wherein the method enriches the base modification in the target polynucleotide between about 300-fold to about 2400-fold compared to whole genome sequencing.

49. The method of claim 1, wherein the adapter-target polynucleotide is eluted from the pulldown complex with a buffer comprising a ribonuclease (RNase).
